(19) United States
(12) Patent Application Publication
(10) Pub. No.: US 2026/0132373 A1
(43) Pub. Date: May 14, 2026

(54) BOVINE BLASTOCYST LIKE STRUCTURES AND USES THEREOF

(71) Applicants: Board of Supervisors of Louisiana State University and Agricultural and Mechanical College, Baton Rouge, LA (US); The Board of Regents of the University of Texas System, Austin, TX (US)

(72) Inventors: Zongliang Carl JIANG, Baton Rouge, LA (US); Yinjuan WANG, Baton Rouge, LA (US); Carlos A. PINZON-ARTEAGA, Dallas, TX (US); Jun WU, Dallas, TX (US); Yulei WEI, Dallas, TX (US)

(21) Appl. No.: 19/099,807

(22) PCT Filed: Aug. 1, 2023
(86) PCT No.: PCT/US2023/029234

§ 371 Date: Jan. 30, 2025

Related U.S. Application Data
(60) Provisional application No. 63/370,068, filed on Aug. 1, 2022.
(60) Provisional application No. 63/413,798, filed on Oct. 6, 2022.

(51) Int. Cl. C12N 5/0735; A61D 19/04

(52) U.S. Cl. CPC C12N 5/0606; A61D 19/04; C12N 2501/115; C12N 2501/16; C12N 2501/235; C12N 2501/415; C12N 2501/727; C12N 2503/02; C12N 2513/00

(57) ABSTRACT

The present invention is directed to method of producing a bovine blastoid and uses thereof.

This application is an International Application which claims priority from U.S. Provisional Patent Application No. 63/370,068 filed on Aug. 1, 2022 and U.S. Provisional Patent Application No. 63/413,798 filed on Oct. 6, 2022, respectively, the contents of which are incorporated by reference herein in its entirety.

### GOVERNMENT INTERESTS

This invention was made with government support under Grant No. 2019-67016-29863 awarded by the USDA National Institute of Food and Agriculture. and Grant No. R01HD102533 awarded by the NIH Eunice Kennedy Shriver National Institute of Child Health and Human Development (NICHD). The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

The present invention is directed to method of producing a bovine blastoid and uses thereof.

### SUMMARY OF THE INVENTION

An aspect of the invention is directed to an in vitro method for producing a blastocyst-like structure (blastoid).

In embodiments, the method comprises culturing a bovine embryonic stem cell (bESC) or an induced pluripotent stem cell (iPSC) with a bovine trophoblast stem cell (TSC) for a length of time sufficient to observe the formation of a three-dimensional (blastoid) in a cell culture medium. In embodiments, the method comprises incubating a SOX2-positive bESC or a SOX2-positive iPSC with a TSC expressing CDX2 for a length of time sufficient to observe formation of a SOX17-positive hypoblast in a cell culture medium, wherein the medium comprises one or more factors selected from the group consisting of a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof.

In embodiments, the STAT3 agonist comprises human leukemia inhibitory factor (hLIF).

In embodiments, the SMAD2/3 agonist comprises recombinant activin A (Activin A).

In embodiments, the Wnt agonist comprises a glycogen synthase kinase-3 (GSK-3) inhibitor. For example, the glycogen synthase kinase-3 (GSK-3) inhibitor comprises CHIR99021.

In embodiments, the inhibitor of the MEK/ERK pathway comprises PD0325901.

In embodiments, the fibroblast growth factor is FGF2.

In embodiments, the PI3K-AKT agonist is insulin.

In embodiments, the medium further comprises one or more factors selected from the group consisting of a ROCK kinase inhibitor, a pan-caspase inhibitor, an integrated stress response (ISR) inhibitor, and Ix polyamine supplement.

In embodiments, the ROCK kinase inhibitor is either chroman-1 or Y-27632.

In embodiments, the pan-caspase inhibitor comprises emricasan.

In embodiments, the integrated stress response (ISR) inhibitor comprises trans-ISRIB.

In embodiments, the method further comprises isolating the resulting bovine blastoid from the culture.

In embodiments, the medium comprises a STAT3 agonist, a SMAD2/3 agonist, a Wnt agonist, an inhibitor of the RAS-RAF-MEK/ERK-MAPK pathway, a fibroblast growth factor, and a PI3K-AKT agonist.

In embodiments, the amount of the STAT3 agonist is about 20 ng/ml.

In embodiments, the amount of the SMAD2/3 agonist is about 10 ng/ml.

In embodiments, the amount of the Wnt agonist is about 1 μM.

In embodiments, the amount of the inhibitor of the MEK/ERK pathway is about 0.3 μM.

In embodiments, the amount of the fibroblast growth factor is about 10 ng/ml.

In embodiments, the amount of the ROCK kinase inhibitor is about 50 nM.

In embodiments, the amount of the pan-caspase inhibitor is about 5 μM.

In embodiments, the amount of the integrated stress response (ISR) inhibitor is about 0.7μ M.

In embodiments, the STAT3 agonist is human leukemia inhibitory factor (hLIF), the SMAD2/3 agonist is recombinant activin A (Activin A), the Wnt agonist is CHIR99021, the inhibitor of the RAS-RAF-MEK/ERK-MAPK pathway is PD0325901, the fibroblast growth factor is FGF2, and the PI3K-AKT agonist is insulin.

In embodiments, the culturing is conducted in a microwell plate.

In embodiments, the period of time is sufficient to transform the bovine embryonic stem cell and trophoblast stem cell into a bovine blastoid, wherein the blastoid mimics bovine blastocyst morphology, bovine blastocyst size, bovine blastocyst cell number, bovine blastocyst gene marker expression, bovine blastocyst lineage composition, bovine blastocyst allocation, or any combination thereof. For example, the period of time is about 3 days.

In embodiments, the method further comprises co-culturing the embryonic stem cell with a trophoblast stem cell. For example, the trophoblast stem cell comprises a bovine trophoblast stem cell.

In embodiments, the embryonic stem cell is an induced embryonic stem cell derived from a somatic cell.

Aspects of the invention are further drawn towards a method of assisted reproduction of a subject.

In embodiments, the method comprises implanting the bovine blastoid produced by an embodiments as described herein in a subject's uterus.

In embodiments, the bovine blastoid derives from an embryonic stem cell isolated from the subject. For example, the subject is a surrogate.

Aspects of the invention are still further drawn to a method of determining a drug toxicity.

In embodiments, the method comprises contacting the bovine blastoid produced by an embodiment as described herein with a drug, and detecting a toxicity indicator. For example, the toxicity indicator comprises cell death, loss of blastoid cell organization, blastoid growth arrest, development arrest, or any combination thereof.

Aspects of the invention are also drawn towards a bovine blastoid produced by an embodiment as described herein.

In embodiments, the bovine blastoid recapitulates bovine embryo development in vitro and in vivo.

In embodiments, the bovine blastoid is capable of generating a viable embryo.

In embodiments, the bovine blastoid is capable of generating a viable embryo in a surrogate.

In embodiments, the bovine blastoid can generate a viable embryo is indicated by the presence of embryonic stem cells, extraembryonic stem cells, or both. For example, generating a viable embryo is indicated by organization into peri-implantation embryo-like structures in vitro.

In embodiments, the bovine blastoid enters elongation in utero.

Still further, aspects of the invention are drawn towards a cell culture comprising a population of bovine embryonic stem cells in a medium.

In embodiments, the medium comprises one or more factors selected from the group consisting of a STAT3 agonist, a SMAD2/3 agonist, a Wnt agonist, an inhibitor of the RAS-RAF-MEK/ERK-MAPK pathway, a fibroblast growth factor, a PI3K-AKT agonist, or any combination thereof.

In embodiments, the medium further comprises one or more factors selected from the group consisting of a ROCK kinase inhibitor, a pan-caspase inhibitor, an integrated stress response (ISR) inhibitor, and 1× polyamine supplement.

For example, the amount of the STAT3 agonist is about 20 ng/ml.

For example, the amount of the SMAD2/3 agonist is about 10 ng/ml.

For example, the amount of the Wnt agonist is about 1 μM.

For example, the amount of the inhibitor of the MEK/ERK pathway is about 0.3 μM.

For example, the amount of the fibroblast growth factor is about 10 ng/ml.

For example, the amount of the ROCK kinase inhibitor is about 50 nM.

For example, the amount of the pan-caspase inhibitor is about 5 μM.

For example, the amount of the integrated stress response (ISR) inhibitor is about 0.7 μM.

In embodiments, the STAT3 agonist is human leukemia inhibitory factor (hLIF), the SMAD2/3 agonist is recombinant activin A (Activin A), the Wnt agonist is CHIR99021, the inhibitor of the RAS-RAF-MEK/ERK-MAPK pathway is PD0325901, the fibroblast growth factor is FGF2, and the PI3K-AKT agonist is insulin.

In embodiments, the culture is in a microwell plate.

In embodiments, the cell culture further comprises a population of trophoblast stem cells. For example, the population of trophoblast stem cells comprises a population of bovine trophoblast stem cells.

Other objects and advantages of this invention will become readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 provides a representation of the bovine embryonic stem cells (bESCs). Panel A provides a schematic diagram showing the transition of primed bESC into naive like bESC m a LCDM condition (bESCLCDM). Panel B provides IF staining images of SOX2. CDX2, and GATA3 in ESCs. Panel C provides a schematic depicting a PCA plot of RNA-seq data from bovine ESCs (LCDM-ESCs, primed ESCs, EPSCs), TSCs, and ICM of bovine blastocysts. These data indicate LCDM-bESCs exhibit naive like pluripotency features.

FIG. 2 provides a representation of the bovine trophoblast stem cells (bTSCs). Panel A provides a schematic diagram representing derivation of bTSC. Panel B provides a representation of brightfield images of bTSC. D5: outgrowth at day 5; P: passage (Scale bar: 100 μm). Panel C provides a representation of IF staining for GATA3, KRT8, CDX2 and SOX2 in bTSCs and IVF blastocysts (Scale bar: 50 μm). Panel D provides a representation of RT-PCR analysis of trophoblast markers in bTSCs. Panel E provides a representation depicting flow cytometry analysis of GAT A3 in bTSC. In vitro (Panel F) and in vivo (Panel G) bTSCs developmental potential as evaluated by interferon tau activity: IFNT, binuclear cells, expression of trophoblast markers (PTGS2 and PL-I) and in vivo engraftment of bTSCs into NOD SCID mice. Panel H provides a schematic depicting PCA plot of RNA seq data from TSCs and ESCs among bovine, human and mouse. Panel I provides a schematic depicting PCA plot of RNA-seq data from bTSCs, bTSCs, TE of blastocyst (D7 TE), and trophoblast cells from day 14 embryos (D14_TE). Panel J provides a schematic depicting the expression pattern of trophoblast and pluripotency markers in bTSC, TE, blastocyst, bESC and bEPSC. Panel K provides a schematic of a heatmap of highly enriched signaling pathways in bTSC. Panel L provides a schematic depicting ATAC-seq analysis reveals the enriched motifs in bTSCs.

FIG. 3 provides a representation of the bovine blastoids generated from ESCs and TSCs. Panel A provides a representation showing bovine ESC and TSC lines used to induce into blastoids. Panel B provides a schematic depicting bovine blastoid formation either from directly assembly of bESC and TSC or self-organization of bESC. Right panel showing the representative phase-contrast images of bovine blastoids from both approaches. Scale bars. 100 um. Panel C provides a representation of immunofluorescence co-staining images of TE markers (GATA3 or CDX2 or KRT18). EPI marker (SOX2). PE markers (GATA6 or SOX17), and ZOI and phalloidin in the blastoids. Panel D provides a representation showing immunostaining against CDX2, SOX17, and SOX2 (left panel), and quantification (left panel) of the relative contribution of the three lineages of blastoids vs. IVF blastocysts using Spinning disk confocal microscope.

FIG. 4 provides a representation of in vitro growth of bovine IVF blastocysts and blastoids. Representative brightfield stereomicroscopic images (left panel) and the diameters (right panel) of in vitro cultured embryos developed in four different tested conditions [Elongation medium 36 without (Panel A) or with (panel B) agarose coated plate: TDM3 medium 12 without (Panel C) or with (Panel D) agarose coated plate]. Data shows that all conditions except for condition 1 could sustain bovine embryos in vitro for a longer period. (Panel E) provides a representation of brightfield stereomicroscopic images (left panel) and the diameters (right panel) of in vitro cultured bovine blastoids developed in two tested conditions (NAR: N2B27 basal medium supplement with activinA and ROCKi; NARFF; N2B27 basal medium supplement with activinA, ROCKi, bFGF and IGFI)36. Scale bar: um.

FIG. 5 provides a representation of the scRNA-seq analysis performed. bovine pre-(day 8 blastocyst, Panel A) and peri-implantation embryo development at day 12 (Panel B), 16 (Panel C), and 18 (Panel D) elongated embryos. The clustering analysis showing the lineage development of epiblast, hypoblast, and trophectoderm cells. A combination of clustering analysis, pathway enrichment analysis, and their known lineage marker gene expression were used to annotate the identities of cell lineages.

FIG. 6 provides a representation of the assembly of bovine blastoids from EPSC and TSC cultures. Panel A provides an illustration of the assembly process via bovine EPSC and TSC aggregation. Panel B provides a phase-contrast image comparing blastoids vs. blastocysts. Panel C provides blastocele diameter measurement: number represents unpaired t test p value. Panel D provides inner cell mass (ICM) diameter measurement: number represents unpaired t test p value. Panel E provides immunostaining for epiblast marker SOX2 (magenta. EPI), hypoblast marker SOX17 (red. HYPO), and trophectoderm marker CDX2 (green. TE), individual markers provided in FIG. 8. Panel F provides a blastoid heatmap pseudo color on quantified spots using IMARIS software for SOX2, CDX2, and SOX17. Panel G provides blastocyst and blastoid lineage composition quantified via confocal microscopy 3D reconstruction and spots colocalization quantification using IMARIS. Panel H provides snapshots of in vitro growth of blastoids in a rotating culture system (Clinostar Incubator, Celvivo). Panel I provides a representative image via immunostaining of all three lineages as in Panel E, individual markers provided in FIG. 8. Panel J provides blastoid diameter quantification. Panel K provides representative micrographs of in vitro grown blastoid. Panel L provides a schematic of the maternal recognition of the action of pregnancy signal interferon-tau (INFt). Panel M provides enzyme-linked immunosorbent assay (ELISA) measurement of (INFt) in surrogate recipients following embryo transfers. PGF2a, prostaglandin F2a; CL, corpus luteum; P4, progesterone, numbers represent unpaired t test p value.

FIG. 7 provides a representation of single-cell characterization of bovine assembled blastoids. Panel A provides joint uniform manifold approximation and projection (UMAP) embedding of 103 Genomics single-cell transcriptomes of bovine blastoids (gray) and bovine zygote (pink), 2 cell (orange). 8 cell (blue), 16 cell (green), morula (cyan), and in vivo and in vitro blastocyst stage embryos (purple, dark green, and light red). Panel B provides UMAP Heatmap showing expression of trophectoderm (TE), hypoblast (HYPO), and epiblast (EPI) markers. GATA2, SOX17, and SOX2, respectively. Panel C provides principal component analysis (PCA) of pseudo bulk conversion of blastoid data. Gastrulation markers32: Disc, embryonic disc (day 14 stage 4); EmE, embryonic ectoderm (day 14 stage 5); MEH, mesoderm, endoderm, and visceral hypoblast (day 14 stage 5): PH, parietal hypoblast (day 14 stage 5); TB, trophoblast (day 14 stage 5). Panel D provides PCA heatmaps showing expression of trophectoderm (TE), hypoblast (HYPO), and epiblast (EPI) markers, GATA3, SOX17, and OCT4 (also known as POU5F1), respectively. Panel E provides major cluster classification based on marker expression. Panel F provides normalized percentage of cells in each cluster. Panel G provides a dot plot indicating the expression of markers of epiblast (EPI), trophectoderm (TE), and hypoblast (HYPO). Panel H provides a violin plot of lineage-specific cell junction markers. Panel I provides RNA velocity pseudotime analysis depicting the cell trajectories.

FIG. 8 provides a representation of media optimization and blastoid characterization. Panel A provides immunostaining of bovine EPSC and TSCs for epiblast marker SOX2 (cyan), hypoblast marker SOX17 (red) and trophectoderm marker CDX2 (green). Panel B provides quantification of blastoid formation efficiency and representative image. Immunostaining and quantification of epiblast marker SOX2 (magenta), hypoblast marker SOX17 (red) and trophectoderm marker CDX2 (green) in (Panel C) FACL, (Panel E) tFACL, and (Panel G) FACL+PD. Quantifications in (Panels D-H) n=2, mean±s.d. Immunostaining for SOX2 (magenta), SOX17 (red) and CDX2 (green) of (Panel I) IVF Blastocysts and (Panel J) Blastoids, arrows depict marker color scheme in Panel L. Panel K provides DAPI normalized relative intensity quantification of side-by-side staining and imaging of blastocysts and blastoids n=5, mean±s.d. Panel L provides blastocyst and blastoid lineage composition quantified via confocal microscopy 3D reconstruction and spots colocalization quantification using IMARIS. (Panel M) Blastocyst and (Panel N) Blastoid immunostaining for epiblast marker SOX2 and trophectoderm markers gata3, Keratin 18: phospho-STAT3 (Blastocyst), and tight junction marker ZO1 (TJP1) and apical marker F-actin (Phallodin) (Blastoid). Blastoids lineage quantification for epiblast marker SOX2 (AF-647), hypoblast marker SOX17 (AF-555, DsRed channel) and trophectoderm marker CDX2 (AF-488. GFP channel). Panel O provides unstained control (Panel P) days 3 and 4 of protocol together with tSNE plots of each of the quantified markers. Panel Q provides lineage quantification via flow cytometry averages n=3, mean±s.d. Panel R provides imaging examples of stained cells used in FACS quantification. Panel S provides immunostaining of bovine blastoids grown in the 3D ClinoStar incubator at day 16 for epiblast marker SOX2 (magenta), hypoblast marker SOX17 (red) and trophectoderm marker CDX2 (green) in tFACL+PD media. Panel T provides a phase-contrast image of bovine blastoids grown in the ClinoStar incubator. Panel U provides images of bovine IVF blastocyst grown in in the ClinoStar incubator at day 16 for stained as in Panel S. Panel V provides a phase-contrast image of in vitro grown bovine blastocyst. Panel V and Panel W provide quantification of in vitro grown blastoids and blastocysts on N2NB27 with rock inhibitor (Y27632) and activin A.

FIG. 9 provides single-cell RNA-seq characterization of blastoids. Panel A provides principal component analysis (PCA) heatmaps of first pseudo bulk conversion of blastoid data colored by dataset and heatmaps of Panel B provide epiblast markers. NANOG. POU5F1 (OCT4): (Panel C) hypoblast markers: SOX17, GATA4; and (Panel D) trophectoderm markers: GATA3, GATA2. (Panel E) UMAP of blastoid 10× data and clustering analysis. Cluster allocation and heatmaps of (Panel F) trophectoderm marker (GATA2). (Panel G) epiblast marker POU5F1 (OCT4), (Panel H) epiblast to hypoblast transition marker RSPO3, and (Panel I) hypoblast marker SOX17. Panel J provides principal component analysis (PCA) of second pseudo bulk conversion of blastoid data based on the 52 clusters of Panel E, showing annotations for datasets, developmental stages, cell types and assigned lines. Panel K provides a dot plot indicating the expression of markers of pre lineage cells, epiblast (EPI), epiblast to hypoblast transitioning cells (E>H), trophectoderm (TE) and hypoblast (HYPO). Heatmaps of (Panel L), epiblast markers: POU5F1 (OCT4) and SOX2; (Panel M) trophectoderm markers: GATA3. GATA2; and (Panel N) hypoblast markers: SOX17. GATA4. Panel O provides a violin plot heatmap of pluripotency related genes in three datasets. Panel P provides a violin plot heatmap of signaling pathways key genes. Panel Q provides UMAP of trophectoderm subclusters and heatmaps of epiblast marker POU5F1 (OCT4) within the TSC subcluster indicating an early blastocyst like subpopulation, and INFt transcript INFT2 expression within TSC subcluster. Panel R provides an expression heatmap and pseudotime analysis of different markers. Panel S provides an alluvial diagram of Go pathways and KEGG pathway of differentially expressed genes (DEG) in each cluster. Panel T provides UMAP of Human blastoid, blastocyst and bovine blastoid scRNA-seq comparison. Panel U provides a violin plot heatmap of pluripotency related genes in three datasets. Panel V provides a violin plot heatmap of signaling pathways key genes.

FIG. 10 provides a representation showing bovine blastocysts produced by in vitro fertilization (IVF). Panel A provides an image taken at 0 seconds. Panel B provides an image taken at 4 seconds. Panel C provides an image taken at 8 seconds. Panel D provides an image taken at 12 seconds. Panel E provides an image taken at 16 seconds.

FIG. 11 provides a representation showing trophoblast cells and cavities in both IVF blastocysts and blastoids continued to proliferate and expand for more than two weeks, which were also accompanied by an increase in inner cell mass size. Panel A provides an image taken at 0 seconds. Panel B provides an image taken at 3 seconds. Panel C provides an image taken at 6 seconds. Panel D provides an image taken at 9 seconds. Panel E provides an image taken at 12 seconds.

FIG. 12 shows the identified cell clusters based on marker gener expression.

FIG. 13 provides principal component analysis (PCA) heatmaps of pseudo bulk conversion of blastoid data. Panel A provides color by dataset. Epiblast markers are provided as follows: (Panel B) NANOG. (Panel C) POU5F1 (OCT4). (Panel D) PRDM14. Hypoblast markers are provided as follows: (Panel E) SOX17. (Panel F) GATA4. (Panel G) PDGFRA. (Panel H) FN1. (Panel I) HNF4A. (Panel J) HNF1B. (Panel K) FOXA2. (Panel L) COL4A1. (Panel M) MSX2. Trophectoderm markers are provided as follows: (Panel N) GATA3. (Panel O) GATA2. (Panel P) DAB2. (Panel Q) KRT19. (Panel R) OVOLI (Panel S) GRHL1.

FIG. 14 provides blastoid TS and ES sub clustering analysis. Panel A provides UMAP of blastoid data and clustering analysis. Panel B provides cluster allocation. Panel C provides a heatmap of epiblast marker POU5F1 (OCT4). Panel D provides a heatmap of trophectoderm marker (GATA3). Panel E provides a heatmap of hypoblast marker SOX17. Panel F provides UMAP of epiblast subclusters. Panel G provides a violin plot comparison of different signaling markers. Panel H provides a violin plot comparison of different pluripotency markers. Panel I provides UMAP of trophectoderm subclusters. Panel J provides a violin plot comparison of different signaling markers. Panel K provides a violin plot comparison of different pluripotency markers. Panel L provides a heatmap of epiblast marker POU5F1 (OCT4) within the TSC subcluster indicating an early blastocyst like subpopulation. Panel M provides a heatmap of INFt transcript INFT2 expression within TSC subcluster.

FIG. 15 provides RNA velocity and pathway analysis. Panels A-O provide expression heatmap and pseudotime analysis of different markers. Panels P-U provide alluvial diagrams of Go pathways of differentially expressed genes (DEG) in each cluster s-u. Alluvial diagram of KEGG pathway of DEGs.

FIG. 16 provides human blastoid, blastocyst and bovine blastoid scRNA-seq comparison. Panels A-C provide UMAP of data integration. Panel D provides a heatmap of trophectoderm marker (GATA3). Panel E provides a heatmap of epiblast marker POU5F1 (OCT4). Panel F provides a heatmap of hypoblast marker SOX17. Panel G provides a violin plot comparison of different signaling markers. Panel H provides a violin plot comparison of different pluripotency markers.

FIG. 17 provides a schematic showing a method for the efficient generation and in vitro growth of bovine blastocyst-like structures (blastoids) from stem cell cultures.

FIG. 18 provides bovine embryo transfer data. Panel A provides a timeline of synchronization experiments, transfers, and flushing. Panel B shows IVF and in vivo embryos recovered at different stages of development. Panel C shows blastoid derived structures. Panel D provides immunostaining characterization of trophectoderm (PTGS2), hypoblast (GATA6) and mesoderm (T) markers.

FIG. 19 provides a representation showing bovine pluripotent stem cells. Panel A provides a shematic showing the three ways bovine stem cells have been derived, by blastocyst derivation, scomatic cell nuclear transfer or transcription factor mediated reprograming (Oct4, Sox2, Klf4, C-Myc) in combination with epigenetic modiers. Throphecoterm (TSCs) and Extra embryonic endodem (XEN) stem cells can also be derived. Panel B provides a schematic showing the expectrum of pluripotency and the currently published culture conditions.

FIG. 20 provides a representation showing self-organized and assembled Blastoids from PSCs cultures. Panel A provides immunostaining of bovine expanded embryonic stem cells (ESCs) and Trophectoderm stem cells (TSCs). Panels B-D provides a schematic showing the approach for the assembly of blastoid formation. Panels E-F provide micrographs of Bovine ESCs self organization blastoids formation. Panel G provides micrographs and videos of assembled or self organized blastoids.

FIG. 21 provides a representation showing the characterization of blastoids. Panel A provides a graph showing size measurements in aggregation medium. Panel B provides a graph showing size measurements in maturation medium. Panels C-D provide immunostaining images. Panels E provides graphs showing marker distribution quantification. Panel F provides immunostaining images.

FIG. 22 provides a representation showing the characterization of blastoids. Panel A provides images showing de novo line derivation of self organized Blastoids and Throphospheres. Panel B provides a representation showing 2D differentiation of bovine ESCs to TSCs.

FIG. 23 provides a representation showing in vitro growth of blastoids. Panel A provides images showing Day 6 of in vitro growth. Panels B-C provide graphs showing size measurements. Panels D-E provide immunostaining of IVF blastocyst (Panel D) and TS:EPS blastoid (Panel E).

FIG. 24 provides a schematic showing the measurement of Interferon-Tau in blood of surrogates cows. Data representing an ELISA assay of (IFN-1) in the blood of synchronized surrogates after embryo transfer is shown.

FIG. 25 provides a schematic showing signaling pathways involved in blastoid formation.

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”. “includes.” “has.” and “involves”) and the like are used interchangeably and have the same meaning. Specifically, each of the terms is defined consistent with the common United States patent law definition of “comprising” and is therefore interpreted to be an open term meaning “at least the following.” and is also interpreted not to exclude additional features, limitations, aspects, etc. Thus, for example, “a process involving steps a, b, and c” means that the process includes at least steps a, b, and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are drawn to methods of culturing, expanding, or growing a population of cells derived from a mammalian blastocyst. Once fertilized, a zygote travels down the fallopian tube and mitotically divides many times to form a population of cells called a blastocyst. The blastocyst consists of an inner mass that develops into the embryo, while the outer layer develops into tissue that nourishes and protects the embryo. The blastocyst attaches onto the wall of the uterus and receives nourishment through the mother's blood. The major systems structures of the calf develop during the embryonic period in a process called differentiation. During this stage, kidney, brain, spinal cord, nerve, heart, and blood cells start to develop, and the gastrointestinal tract begins to form.

Trophoblasts are cells that form the outer layer of a blastocyst. They provide nutrients to the embryo and develop into a large part of the placenta. They form during the first stage of pregnancy and are the first cells to differentiate from the fertilized egg to become extraembryonic structures and do not directly contribute to the embryo.

Embodiments as described herein comprise culturing cells derived from a mammalian blastocyst, for example, a bovine blastocyst. “Culturing” a cell or a population of cells can refer to propagating or nurturing a cell, collection of cells, tissue, or organ, by incubating for a period of time in an environment and under conditions which support cell viability or propagation. For example, culturing a cell can maintain the cell under conditions in which it can proliferate, differentiate, and avoid senescence. For example, the environment and conditions which support cell viability and/or propagation can include culturing a cell or a population of cells in a culture medium comprising a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof.

Culturing can include one or more of the steps of expanding and proliferating a cell or population of cells, and/or collecting a cell, a population of cells, a tissue, or and organ.

“Expanding” a population of cells can refer to culturing the cells for a period of time and under conditions that not only allow the cells to grow and develop, but also proliferate, so that at the end of the expansion, more cells are obtained than before the expansion. For example, one cell can be expanded by cell division to two cells. In embodiments, expansion of a population of cells can occur spontaneously as certain cells proliferate in a culture. In other embodiments, expansion of a population of cells can require certain growth conditions, including but not limited to a minimum cell density, cell confluence on the culture vessel surface, or the addition of chemical factors such as growth factors, differentiation factors, or signaling factors.

As described herein, embodiments comprise culturing cells derived from a mammalian blastocyst. As used herein, “mammal” or “mammalian” can refer to any mammal, non-limiting examples of which include a human, a primate, mouse, rat, dog, cat, bovine, cow, horse, pig, a fish, or a bird. For example, “bovine” can refer to an animal from the cattle group, non-limiting examples of which include cows, buffalo, and bison.

In embodiments, the method described herein comprises culturing mammalian cells in a culture medium. The terms “medium”, “cell culture medium”, “culture medium” can refer to a solution containing nutrients that nourish growing cells. In certain embodiments, the culture medium is useful for growing mammalian cells. A culture medium can provide essential and non-essential amino acids, vitamins, energy sources, lipids, and trace elements required by the cell for minimal growth and/or survival. A culture medium may also contain supplementary components (see discussion of “Supplementary components” below) that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source. In certain embodiments, a medium is advantageously formulated to a pH and salt concentration optimal for cell survival and proliferation.

In embodiments, the culture medium can comprise “supplementary components”, which can refer to components that enhance growth and/or survival above the minimal rate. Non-limiting examples of supplementary components include hormones and/or other growth factors, ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source. In certain embodiments, supplementary components are added to the initial cell culture. In certain embodiments, supplementary components are added after the beginning of the cell culture.

The term “defined medium” can refer to a medium in which the composition of the medium is both known and controlled. See, for example, the medium of Example 4.

In embodiments, the cell culture can comprise a “nutrient source”, which can refer to a composition, including the source itself, that nourishes growing mammalian cells. Non-limiting examples of nutrient sources comprise DMEM, IDMEM, MEM, M199, RPMI 1640, Ham's F12, DMEM/F12, Ham's F10, McCoy's 5A, NCTC 109, and NCTC 135.

A “culture medium” can refer to a solution for growing, storing, handling and maintaining a cell, a population of cells, and/or cell lines. In embodiments, solutions can include factors required for or assist with cell attachment, cell growth, cell proliferation, maintenance of a cell in an undifferentiated state, and/or maintenance of the cellular environment. Non-limiting examples of such factors include salts, nutrients, minerals, vitamins, amino acids, nucleic acids, proteins such as cytokines, growth factors and hormones.

In embodiments, the culture medium comprises a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof.

In embodiments, the STAT3 agonist is human leukemia inhibitory factor (hLIF), the SMAD2/3 agonist is recombinant activin A (Activin A), the Wnt agonist is CHIR99021, the inhibitor of the RAS-RAF-MEK/ERK-MAPK pathway is PD0325901, the fibroblast growth factor is FGF2, and the PI3K-AKT agonist is insulin.

In embodiments, the medium further comprises one or more factors selected from the group consisting of a ROCK kinase inhibitor, a pan-caspase inhibitor, an integrated stress response (ISR) inhibitor, and 1× polyamine supplement.

In embodiments, the culture medium can be a liquid solution that supports the growth of stem cells, such as trophoblast stem cells, and/or maintains them in an undifferentiated state. For example, a “culture of mammalian cells” can refer to a liquid culture medium containing a plurality of mammalian cells that is maintained or proliferated under a controlled set of physical conditions. In embodiments, the culture medium can be a water-based medium.

A “cell culture” can refer to cells growing in suspension or adhered to a variety of surfaces or substrates in a vessel, such as a roller bottle, tissue culture flask, dish, multi-well plate, and the like. For example, the cell culture can refer to a population of cells, such as trophoblast stem cells, derived from a mammalian blastocyst and a culture medium comprising one or more factors selected from the group consisting of a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof. In embodiments, the medium further comprises one or more factors selected from the group consisting of a ROCK kinase inhibitor, a pan-caspase inhibitor, an integrated stress response (ISR) inhibitor, and 1× polyamine supplement.

In embodiments, the cell culture can comprise a plurality of cells in an undifferentiated state. For example, a cell in an “undifferentiated state” can refer to a cell that does not have specialized structures or functions. Thus, an undifferentiated cell can refer to a cell having differentiation ability that has not entered the process of differentiating into a cell having a specific function as a tissue or organ. In embodiments, the undifferentiated cells are capable of self-renewal.

In embodiments, the cell culture can comprise a plurality of cells capable of differentiation. “Differentiation” or “to differentiate” can refer to to the process by which a less specialized cell (e.g., stem cells, embryonic cells) undergoes maturation to become more distinct in form and function, such as to acquire specialized structural and/or functional features characteristic of mature cells. For example, the less specialized cell (e.g., a stem cell or cell maintaining stemness) can progress from the stage of having the potential to differentiate into a cell of different cellular lineages to the stage of becoming a specialized and terminally differentiated cell. During differentiation, cellular structure alters and tissue-specific proteins appear.

Non-limiting examples of undifferentiated cells include pluripotent stem cells, embryonic stem cells, progenitor cells, induced pluripotent stem cells, germ stem cells, and the like.

Embodiments as described herein provide methods of culturing a population of cells derived from a mammalian blastocyst, wherein the population of cells maintain cell viability. “Cell viability” can refer to the ability of cells in culture to survive under a given set of culture conditions or experimental variations. The term can also refer to that portion of cells which are alive at a particular time in relation to the total number of cells, living and dead, in the culture at that time.

In embodiments, the method as described herein can comprise placing a mammalian blastocyst in a vessel, thereby providing a vessel comprising a poplution of cells derived from a mammalian blastocyst. Any suitable vessel can be used in the embodiments described herein. For example, a “vessel” can refer to a type of culture ware which provides a contamination barrier to protect the cultures from the external environment while maintaining the proper internal environment. Non-limiting examples of a vessel comprise a flask, a tube, a Petri dish, a roller bottle, and/or a multi-well plate.

For example, the population of cells derived from a mammalian blastocyst, such as a population of trophoblast stem cells, can be cultured in a microwell plate. In some cases, the microwell plate is a v-bottomed microwell plate. In some cases, the v-bottomed microwell plate is an AggreWell plate.

In embodiments, culturing a population of cells as described herein can comprise centrifuging a culture vessel comprising the population of cells and the culture media. For example, a culture vessel can be centrifuged at about 50× g after the cell and medium is added to the plate. For example, a culture vessel can be centrifuged at about 100× g after the cell and medium is added to the plate. For example, a culture vessel can be centrifuged at about 150× g after the cell and medium is added to the plate. For example, a culture vessel can be centrifuged at about 200× g after the cell and medium is added to the plate. For example, a culture vessel can be centrifuged at about 250× g after the cell and medium is added to the plate. For example, a culture vessel can be centrifuged at about 300× g after the cell and medium is added to the plate. For example, a culture vessel can be centrifuged at about 350× g after the cell and medium is added to the plate. For example, a culture vessel can be centrifuged at about 400× g after the cell and medium is added to the plate. For example, a culture vessel can be at about 450× g after the cell and medium is added to the plate. In some cases, the v-bottomed plate is centrifuged at about 500× g after the cell and medium is added to the plate.

In embodiments, the vessel can be seeded with fibroblast cells, such as mouse embryonic fibroblast cells. “Cell seeding” can refer to spreading cells into or onto a surface of a vessel. “Fibroblast cells” can refer to a cell that contributes to the formation of connective tissue and are not terminally differentiated. Fibroblasts are heterogeneous mesenchymal cells that play important roles in the production and maintenance of extracellular matrix.

In embodiments, the trophoblast stem cells can be cultured with or without fibroblast feeder cells. A “fibroblast feeder cell” can refer to a cell of one type that can be cultured with a cell of another type to provide an environment in which the cell of the second type can grow. For example, a trophoblast stem cell can be cultured with a MEF such that the trophoblast stem cell grows. The feeder cell can be a human feeder cell or can be a non-human feeder cell. In one embodiment, the feeder cell can be a mouse embryonic fibroblast.

The phrase “feeder cell support” as used herein refers to the ability of a feeder cell (e.g., fibroblasts) to maintain pluripotent stem cells in a proliferative and undifferentiated state when the pluripotent stem cells are co-cultured on the feeder cells or when the pluripotent stem cells are cultured on a matrix (e.g., an extracellular matrix, a synthetic matrix) in the presence of a conditioned medium generated by the feeder cells. The support of the feeder cells depends on the structure of the feeder cells while in culture (e.g., the three dimensional matrix formed by culturing the feeder cells in a tissue culture plate), function of the feeder cells (e.g., the secretion of growth factors, nutrients and hormones by the feeder cells, the growth rate of the feeder cells, the expansion ability of the feeder cells before senescence) and/or the attachment of the pluripotent stem cells to the feeder cell layer(s).

The phrase “absence of feeder cell support” as used herein refers to a culture medium and/or a cell culture being devoid of feeder cells and/or a conditioned medium generated thereby.

In embodiments, the vessel can comprise a “surface” to which the cell or population of cells can attach. For example, the surface can be a solid substrate, a porous substrate, or another non-solid substrate.

For example, the solid surface can be coated with an insoluble substrate that, optionally, can in turn be coated with one or more additional surface coats of a substrate, or any other chemical or biological material that allows the cells to proliferate or be stabilized in culture. Non-limiting examples of a substrate comprise any one or combination of polyornithine, laminin, poly-lysine, purified collagen, gelatin, fibronectin, tenascin, vitronectin, entactin, heparin sulfate proteoglycans, polyglycolytic acid (PGA), polylactic acid (PLA), and polylactic-glycolic acid (PLGA).

A “porous surface”, for example, can refer to a surface which allows for the partitioning of cellular microenvironments in vitro, while still allowing physical and biochemical crosstalk between cells.

In embodiments, the cells can be cultured on a surface coated with extracellular matrix or a component of extracellular matrix as described herein.

As described herein, aspects of the invention are drawn towards culturing a population of cells derived from a mammalian blastocyst. The terms “cell” and “population of cells” can refer to a plurality of cells (i.e., more than one cell). In embodiments, the population can be a pure population comprising one cell type. In other embodiments, the population can include multiple cell types. Accordingly, there is no limitation on the types of cells that the population of cells can contain. In embodiments, the population of cells can comprise trophoblast stem cells or trophoblast stem-like cells.

Any suitable population of cells can be used in methods for culturing, expanding, or growing a population of cells derived from a mammalian blastocyst, such as trophoblast stem cells as described herein. In embodiments, the population of cells can comprise reproductive cells, e.g., female germline stem cells and progeny thereof. Examples of reproductive cells include, but are not limited to, embryos, oocytes, zygotes, blastomeres, morulae, and blastocysts.

In embodiments, the population of cells can comprise somatic cells such as fibroblasts (e.g., embryonic fibroblasts or skin fibroblasts). Somatic cells can be obtained by well-known methods from different organs. e.g., skin, lung, pancreas, liver, stomach, intestine, heart, reproductive organs, bladder, kidney, urethra, and other urinary organs. Examples of somatic cells include, but are not limited to, adult stem cells, Sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B lymphocytes and T lymphocytes), erythrocytes, macrophages, monocytes, mononuclear cells, fibroblasts, cardiac muscle cells, and other muscle cells.

The suitable population of cells can be obtained from any suitable source. In embodiments, the population of cells can be obtained from a subject, for example, from tissue (e.g., embryotic tissue), bone (e.g., bone marrow), blood (e.g., peripheral blood or umbilical cord blood), bodily fluid (e.g., tear, urine, or saliva), serum, plasma, or protein, from a subject via any means known in the art. A subject includes, but is not limited to, a human or a non-human mammal such as a rodent (e.g., a mouse or a rat), an ungulate (e.g., a horse or a pig), or bovine (e.g., cow).

A “stem cell” can refer to an undifferentiated cell which is capable of essentially unlimited propagation either in vivo or ex vivo and capable of differentiation to other cell types. This can be to certain differentiated, committed, immature, progenitor, or mature cell types present in the tissue from which it was isolated, or dramatically differentiated cell types that derive from a common precursor cell, or even to cell types at any stage in a tissue completely different from the tissue from which the stem cell is obtained. A stem cell can retain a constant potential for differentiation even after undergoing cell division. Examples of the stem cells include embryonic stem cells (ES cells) with pluripotency derived from a fertilized egg or a clone embryo, somatic stem cells and pluripotent stem cells that are present in tissues in a live body, hepatic stem cells, dermal stem cells, and germ stem cells that serve as the bases for respective tissues, pluripotent stem cells derived from a germ stem cell, pluripotent stem cells derived from a somatic cell that are obtained by nuclear reprogramming, and the like.

A “stem-like cell” can refer to cells that have some of the characteristics of stem cells. For example, they have some ability to self-renew. Examples of stem-like cells include, but are not limited to, progenitor cells, multipotent stem cells, cells undergoing process to induce pluripotency, cancer cells, cancer stem cells, hematopoietic stem cells, iPS, and some antibody producing hybridoma cells.

A “trophoblast stem cell” can refer to the precursor of the differentiated cells of the placenta, which mediate the interactions between the fetus and the mother.

A “pluripotent stem cell” can refer to a stem cell that permits cultivation in vitro. A pluripotent stem cell can differentiate into all cells constituting the body. A “pluripotent stem cell” can be obtained from a fertilized egg, a clone embryo, a germ stem cell, or a stem cell in a tissue. Also included are cells having differentiation pluripotency similar to that of embryonic stem cells, conferred artificially by transferring several different genes to a somatic cell.

An “embryonic stem cell” can refer to a cell which is obtained from the embryonic tissue formed after gestation (e.g., blastocyst) before implantation (i.e., a pre-implantation blastocyst), extended blastocyst cells (EBCs) which are obtained from a post-implantation/pre-gastrulation stage blastocyst, and embryonic germ (EG) cells which are obtained from the genital tissue of a fetus any time during gestation, preferably before 10 weeks of gestation. A “fetus” can refer to a mammal in the developmental stage after the embryonic stage and before birth, with fully differentiated but not yet fully grown organs.

Examples of stem cells that can be used in embodiments described herein include mammalian embryonic stem cells or the like established by culturing a pre-implantation early embryo, embryonic stem cells established by culturing an early embryo prepared by nuclear-transplanting the nucleus of a somatic cell, trophoblast stem cells established from various species, including bovine, mouse, human, and nonhuman primates, and induced pluripotent stem cells (iPS cells) established by transferring several different genes to a somatic cell.

In embodiments, the cells are cultured on a surface coated with extracellular matrix or a component of extracellular matrix (ECM). For example, the ECM can be composed of a variety of polysaccharides, water, elastin, and glycoproteins. Non-limiting examples of glycoproteins can comprise collagen, entactin (nidogen), fibronectin, and laminin. ECM can be secreted by connective tissue cells. Different types of ECM are known, each of which comprise different compositions including different types of glycoproteins and/or different combination of glycoproteins. ECM can be provided by culturing ECM-producing cells, for example fibroblast cells, in a vessel prior to the removal of these cells and the addition of isolated tissue fragments or isolated epithelial stem cells, such as a mammalian blastocyst.

Non-limiting examples of extracellular matrix-producing cells comprise chondrocytes, producing mainly collagen and proteoglycans: fibroblast cells, producing mainly type IV collagen; laminin; interstitial procollagens: fibronectin: colonic myofibroblasts producing mainly collagens (type I, III, and V); chondroitin sulfate proteoglycan: hyaluronic acid: fibronectin; and tenascin-C.

In embodiments, ECM can be commercially provided. Non-limiting examples of commercially available extracellular matrices comprise extracellular matrix proteins (Invitrogen) and basement membrane preparations from Engelbreth-Holm-Swarm (EHS) mouse sarcoma cells (e.g., MATRIGEL™ (BD Biosciences)). A synthetic extracellular matrix material, such as ProNectin (Sigma Z378666) can be used. Mixtures of extracellular matrix materials can be used, if desired.

In embodiments, the use of an ECM for culturing stem cells can enhance the long-term survival of the stem cells and the continued presence of undifferentiated stem cells.

Aspects of the invention can be further drawn to an in vitro cell culture comprising a population of cells derived from a mammalian blastocyst and a medium as described herein. In embodiments, the in vitro population of cells are capable of self-renewal. “Self-renewal” can refer to the process by which stem cells perpetuate themselves, such as to replenish mature cells to maintain tissue homeostasis throughout the lifespan of an organism. Self-renewal is division with maintenance of the undifferentiated state. This can require cell cycle control and/or maintenance of multipotency or pluripotency, depending on the stem cell.

Aspects of the invention are further drawn to an isolated cell derived from a mammalian blastocyst. An “isolated cell” can refer to a cell that has been removed from its natural environment, for example, as a part of an organ, tissue, or organism.

Markers (i.e., biomarkers) can be used to identify and isolate different cell types. For example, embodiments as described herein can comprise an isolated cell expressing at least one marker of pluripotency. “Pluripotency” can refer to the ability of a cell to develop into the body or all lineages of the body (i.e., embryo body). For example, a pluripotent cell can develop into the three primary germ cell layers of the early embryo, and therefore into all cells of the adult body. Pluripotent stem cells can undergo self-renewal and give rise to all cells of the tissues of the body. Non-limiting examples of a marker of pluripotency comprise CDX2, SOX2, GATA3, ELF3, TFAP2A, KLF5, KRT8, SFN, DNMT1, DNMT3A, PAG2, PAG11, PAG12, CYP17A1, HSD3B1, HAND1, or any combination thereof. Other exemplary markers the isolated cell can express include a marker of a signaling pathway. Non-limiting examples of a signaling pathway comprise Wnt signaling pathway, the LIF signaling pathway, the HIF-1 signaling pathway, the AKT signaling pathway, the AMPK signaling pathway, or any combination thereof.

In other embodiments described herein, the population of cells can comprise at least one marker of a trophoblast stem cell. Referring to Example 1, for example, the resulting cell lines have TSC characteristics, including but not limited to trophoblast marker gene expression, self-renewal, long-term stable morphology, karyotype, and transcriptomic and epigenomic features). Further, the population of cells can contribute to functional uninucleate and binuclear trophoblasts in vitro and in vivo.

Aspects of the invention are also drawn to methods of evaluating a candidate compound. A “candidate compound” can refer to a compound or agent that is to be tested for an activity of interest.

In embodiments, the method comprises contacting the cell culture or the isolated cell as described herein with an amount of the candidate compound, and evaluating a characteristic of the cell culture or isolated cell. Non-limiting examples of characteristics that can be evaluated comprise cell growth, cell development, differentiation, apoptosis, trophoblast development, trophoblast activity, or any combination thereof.

Aspects of the invention further provide a population of cells that can be used for assembly of artificial blastocysts for various Assisted Reproductive Technology (ART) applications. “Assisted reproductive technology” can refer to technology that assists in achieving pregnancy, including, but not limited to, in vitro fertilization (IVF), embryo transfer (e.g., transfer of embryos at any stage, including blastocysts), gamete intrafallopian transfer (GIFT), tubal embryo transfer (TET), intracytoplasmic sperm injection (ICSI) and intrauterine insemination (IUI).

In embodiments, the trophoblast stem cells can be used for the assembly of an artificial blastoid. A “blastoid” can refer to stem cell-based blastocyst-like structures which resemble blastocysts in terms of morphology, size, cell number, and lineage composition and allocation. A “blastocyst” can refer to a thin-walled hollow structure in early embryonic development that contains a cluster of cells called the inner cell mass from which the embryo arises.

As described herein, the culture medium can comprise a a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof.

In embodiments, the STAT3 agonist is human leukemia inhibitory factor (hLIF), the SMAD2/3 agonist is recombinant activin A (Activin A), the Wnt agonist is a GSK-3 inhibitor, the inhibitor of the RAS-RAF-MEK/ERK-MAPK pathway is PD0325901, the fibroblast growth factor is FGF2, and the PI3K-AKT agonist is insulin. Non-limiting examples of the GSK-3 inhibitor include CHIR99021, CHIR98014, CHIR98023, SB-216763 and SB-415286.

The methods as described herein comprise culturing a population of cells derived from a mammalian blastocyst for a period of time in a culture medium comprising at least a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof. For example, the population of cells can be cultured for a period of time sufficient for the assembly of artificial blastocysts. For example, the period of time can be at least 18 hours, at least about 24 hour, at least 36 hours, at least 48 hours, at least 60 hours, at least 72 hours, at least 84 hours, at least 996 hours, at least about 5 days, at least about 6 days, at least about 7 days, at least about 8 days, or longer. In further aspects of methods of culturing trophoblast stem cells, the population of cells is cultured for an appropriate period of time sufficient to form the blastoid. In some cases, the culturing is conducted for about 1 day, about 2 days, about 3 days, about 4 days, about 5 days, about 6 days, about 7 days, about 8 days, about 9 days, about 10 days, or more as needed. In some cases, the culturing is conducted for about 1-5 days, about 2-6 days, about 3-7 days, about 4-8 days, about 5-9 days, or about 6-10 days.

In embodiments, the STAT3 agonist, the SMAD2/3 agonist, the Wnt agonist, the inhibitor of the RAS-RAF MEK/ERK-MAPK pathway, the fibroblast growth factor, and the PI3K-AKT agonist can be provided in the culture medium in an effective amount. An “effective amount,” “effective dose,” or an “amount effective to,” as used herein, can refer to an amount of an agent that is effective in providing at least one characteristic of trophoblast stem cells (e.g., cell growth, cell development, differentiation, apoptosis, trophoblast development, trophoblast activity, or any combination thereof). Such characteristics can be monitored by conventional methods or can be monitored according to methods described herein. An effective amount can vary depending on, for example, the STAT3 agonist, the SMAD2/3 agonist, the Wnt agonist, the inhibitor of the RAS-RAF MEK/ERK-MAPK pathway, the fibroblast growth factor, and the PI3K-AKT agonist used.

For example, the effective amount of the STAT3 agonist, the SMAD2/3 agonist, the Wnt agonist, the inhibitor of the RAS-RAF MEK/ERK-MAPK pathway, the fibroblast growth factor, and the PI3K-AKT agonist used as described herein can result in an increase in the proportion of cells in the formative stage of pluripotency by at least 10% or more, including, e.g., at least about 20%, at least about 30%, at least about 40%, at least about 50%, at least about 60%, at least about 70%, at least about 80%, at least about 90% or more, as compared to the proportion of cells in the formative stage of pluripotency when the population of cells is cultured without the the STAT3 agonist, the SMAD2/3 agonist, the Wnt agonist, the inhibitor of the RAS-RAF MEK/ERK-MAPK pathway, the fibroblast growth factor, and the PI3K-AKT agonist.

In embodiments, an effective amount of a STAT3 agonist can be between about 0.1 and about 10,000 ng/ml. For example, the effective amount of the STAT3 agonist can be between about 1 and about 10,000 ng/ml, between about 10 and about 10,000 ng/ml, between about 100 and about 10,000 ng/ml, between about 1,000 and about 10,000 ng/ml, between about 5.000 and about 10.000 ng/ml, between about 0.1 and about 5.000 ng/ml, between about 1 and about 5,000 ng/ml, between about 10 and about 5,000 ng/ml, between about 1.000 and about 5,000 ng/ml, or between about 2,500 and about 5,000 ng/ml. In some embodiments, the effective amount of the the STAT3 agonist for the methods described herein can be between 0.1 and 100 μM. In some embodiments, the effective amount of the the STAT3 agonist for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the the STAT3 agonist for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM. For example, the STAT3 agonist can comprise a human leukemia inhibitory factor (hLIF). For example, an effective amount of the STAT3 agonist is about 20 ng/ml.

An effective amount of a SMAD2/3 agonist for the methods described herein can be between 0.1 and 10,000 ng/ml. In some embodiments, the effective amount of the SMAD2/3 agonist for the methods described herein can be between 1 and 10,000 ng/ml, between 10 and 10,000 ng/ml, between 100 and 10,000 ng/ml, between 1,000 and 10.000 ng/ml, between 5.000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml. In some embodiments, the effective amount of the SMAD2/3 agonist for the methods described herein can be between 0.1 and 100 μM. In some embodiments, the effective amount of the SMAD2/3 agonist for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the SMAD2/3 agonist for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM. For example, the SMAD2/3 agonist can comprise recombinant activin A (Activin A) For example, an effective amount of the SMAD2/3 agonist agonist is about 10 ng/ml.

An effective amount of a Wnt agonist for the methods described herein can be between 0.1 and 10.000 ng/ml. In some embodiments, the effective amount of the Wnt agonist for the methods described herein can be between 1 and 10,000 ng/ml, between 10 and 10,000 ng/ml, between 100 and 10.000 ng/ml, between 1,000 and 10,000 ng/ml, between 5.000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml. In some embodiments, the effective amount of the inhibitor of a Wnt agonist for the methods described herein can be between 0.1 and 100 μM. In some embodiments, the effective amount of the Wnt agonist for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the inhibitor of a Wnt agonist for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM. For example, the Wnt agonist can comprise a glycogen synthase kinase-3 (GSK-3) inhibitor. For example, the inhibitor of glycogen synthase kinase-3 (GSK-3) can comprise CHIR99021. For example, an effective amount of the Wnt agonist is about 1 μM.

An effective amount of an inhibitor of the MEK/ERK pathway for the methods described herein can be between 0.1 and 10,000 ng/ml. In some embodiments, the effective amount of the inhibitor of the MEK/ERK pathway for the methods described herein can be between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml. In some embodiments, the effective amount of the inhibitor of the MEK/ERK pathway for the methods described herein can be between 0.1 and 100 μM. In some embodiments, the effective amount of the inhibitor of the MEK/ERK pathway for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the inhibitor of the MEK/ERK pathway for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM. For example, the inhibitor of the MEK/ERK pathway can comprise PD0325901. For example, an effective amount of the inhibitor of the MEK/ERK pathway is about 0.3 μM.

An effective amount of a fibroblast growth factor for the methods described herein can be between 0.1 and 10,000 ng/ml. In some embodiments, the effective amount of the a fibroblast growth factor for the methods described herein can be between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5.000 ng/ml, between 1.000 and 5.000 ng/ml, or between 2.500 and 5.000 ng/ml. In some embodiments, the effective amount of the fibroblast growth factor for the methods described herein can be between 0.1 and 100 M. In some embodiments, the effective amount of the a fibroblast growth factor for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 M, between 0.1 and 20 μM, between 0.1 and 10 μM, between 0.1 and 1 μM, and between 0.1 and 0.5 μM. In some embodiments, the effective amount of the fibroblast growth factor for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM. For example, the fibroblast growth factor can comprise FGF2. For example, an effective amount of the fibroblast growth factor is about 10 ng/ml.

An effective amount of a ROCK kinase inhibitor for the methods described herein can be between 0.1 and 10,000 ng/ml. In some embodiments, the effective amount of the ROCK kinase inhibitor for the methods described herein can be between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5.000 ng/ml. In some embodiments, the effective amount of the ROCK kinase inhibitor for the methods described herein can be between 0.1 and 100 μM. In some embodiments, the effective amount of the ROCK kinase inhibitor for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the ROCK kinase inhibitor for the methods described herein can be between 0.5 and 100 μM, between 1 and 100 μM, between 10 and 100 μM, between 20 and 100 M, between 30 and 100 μM, between 40 and 100 μM, between 50 and 100 μM, between 60 and 100 μM, between 70 and 100 μM, between 80 and 100 μM, and between 90 and 100 μM. In some embodiments, the effective amount of the ROCK kinase inhibitor for the methods described herein can be between 0.1 and 100 nM. In some embodiments, the effective amount of the ROCK kinase inhibitor for the methods described herein can be between 0.1 and 90 nM, between 0.1 and 80 nM, between 0.1 and 70 nM, between 0.1 and 60 nM, between 0.1 and 50 nM, between 0.1 and 40 nM, between 0.1 and 30 nM, between 0.1 and 20 nM, between 0.1 and 10 nM, between 0.1 and 1 nM, and between 0.1 and 0.5 nM. In some embodiments, the effective amount of the ROCK kinase inhibitor for the methods described herein can be between 0.5 and 100 nM, between 1 and 100 nM, between 10 and 100 nM, between 20 and 100 nM, between 30 and 100 nM, between 40 and 100 nM, between 50 and 100 nM, between 60 and 100 nM, between 70 and 100 nM, between 80) and 100 nM, and between 90 and 100 nM. For example, the ROCK kinase inhibitor can comprise either chroman-I or Y-27632. For example, an effective amount of the ROCK kinase inhibitor is about 50 nM.

An effective amount of a pan-caspase inhibitor for the methods described herein can be between 0.1 and 10,000 ng/ml. In some embodiments, the effective amount of the pan-caspase inhibitor for the methods described herein can be between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5.000 ng/ml. In some embodiments, the effective amount of the pan-caspase inhibitor for the methods described herein can be between 0.1 and 100 μM. In some embodiments, the effective amount of the pan-caspase inhibitor for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the pan-caspase inhibitor for the methods described herein can be between 0.5 and 100 μM, between 1 and 100 μM, between 10 and 100 μM, between 20 and 100 μM, between 30 and 100 μM, between 40 and 100 μM, between 50 and 100 M, between 60 and 100 μM, between 70 and 100 μM, between 80 and 100 μM, and between 90 and 100 μM. For example, the pan-caspase inhibitor can comprise emricasan. For example, an effective amount of the pan-caspase inhibitor is about 5 μM.

An effective amount of an integrated stress response (ISR) inhibitor for the methods described herein can be between 0.1 and 10,000 ng/ml. In some embodiments, the effective amount of the integrated stress response (ISR) inhibitor for the methods described herein can be between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5.000 ng/ml, between 1 and 5,000 ng/ml, between 10 and 5,000 ng/ml, between 1.000 and 5.000 ng/ml, or between 2.500 and 5,000 ng/ml. In some embodiments, the effective amount of the integrated stress response (ISR) inhibitor for the methods described herein can be between 0.1 and 100 μM. In some embodiments, the effective amount of the integrated stress response (ISR) inhibitor for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the integrated stress response (ISR) inhibitor for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 M, and between 90 and 100 μM. For example, the integrated stress response (ISR) inhibitor can comprise trans-ISRIB. For example, an effective amount of the integrated stress response (ISR) inhibitor is about 0.7 μM.

In embodiments, the culture medium can be changed after culturing the population of cells for a period of time. For example, the contents of the culture medium can be changed after about 8 hours, about 12 hours, about 16 hours, about 24 hours, about 36 hours, or about 48 hours after culturing the population of cells. In embodiments, the medium is replaced with a medium without the STAT3 agonist. In embodiments, the medium is replaced with a medium without the SMAD2/3 agonist. In embodiments, the medium is replaced with a medium without the Wnt agonist. In embodiments, the medium is replaced with a medium without the inhibitor of the RAS-RAF MEK/ERK-MAPK pathway. In embodiments, the medium is replaced with a medium without the fibroblast growth factor. In embodiments, the medium is replaced with a medium without the PI3K-AKT agonist.

Aspects of the invention are also drawn towards methods for the generation of bovine blastocyst-like structures. For example, embodiments can comprise the assembly of bovine trophoblast stem cells and embryonic stem cells in defined culture conditions. Other embodiments can comprise expanded potential pluripotent stem cells as starting cells and sequential treatments of cocktails of growth factors and inhibitors to allow them to differentiate and self-organize into blastoids.

To generate bovine embryos, current technologies mostly rely on natural mating, assisted reproduction, and somatic cell nuclear transfer (SCNT). A common drawback of all current technologies is their reliance on the use of mature oocytes, which are limited in quantity. In contrast, the stem cell based blastoid approach described herein bypasses the use of mature gametes and can produce essentially unlimited number of blastoids that have the potential to generate viable bovine embryos. Another advantage of the technology is the scalability, which allows for the production of hundreds of thousands, if not more, blastoids within a short period of time.

Somatic cell nuclear transfer allows for the production of an isogenetic copy of an organism by reprograming a somatic nucleus by an anucleated mature oocyte. Progress to apply SCNT to livestock species are slow because of its inherent low efficiency, high costs, and amount of labor required. Aspects of the invention described herein can overcome the limitations of SCNT by allowing nearly limitless production of blastocyst-like structures from stem cell cultures.

Embodiments as described herein solve several major problems with the SCNT technology: 1) they can drastically reduce the costs by generating large quantity of blastoids without the need of oocytes and labor-intensive embryo micromanipulations. 2) They are more efficient than SCNT. The rates of blastocysts formation after SCNT are very low. In contrast, bovine blastocyst-like structures can be generated with efficiency as high as >80% within a short period of time. 3) Because in vitro cultured EPSCs and TSCs are both amenable to sophisticated genetic engineering, the bovine blastoid technology can accelerate the generation of genetically modified animals for pharmaceutical or biomaterials production, xenotransplantation and animal models of human disease. Therefore, embodiments as described herein have clear advantages on the following, 1) generate viable bovine embryos without the need of gametes, 2) efficiency is as high as >80%, and 3) large scale production of blastoids over a short period of time.

Embodiments as described herein provide methods of producing a blastoid, such as a mammalian blastoid. In embodiments, the method comprises obtaining or providing an expanded potential pluripotent stem cell (EPSC). In embodiments, the method comprises culturing the EPS cell in a medium as described herein, for example a medium comprising one or more factors selected from the group consisting of a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof.

In an exemplary embodiment, the method comprises obtaining or providing an embryonic stem cell (ESC); culturing the ESC cell in a medium comprising one or more factors selected from the group consisting of of a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof for a period of time sufficient to provide a blastoid; and isolating the resulting blastoid.

In embodiments, the ESC cells can be cultured in any suitable culture vessel, including those described herein. In some cases, the ESC cell is cultured in a microwell plate. In some cases, the microwell plate is a v-bottomed microwell plate. In some cases, the v-bottomed microwell plate is an AggreWell plate.

Embodiments can comprise centrifuging a culture vessel comprising the ESC and the culture media. In embodiments, the v-bottomed plate is centrifuged at about 50× g, at about 100× g, at about 150× g, at about 200× g, at about 250× g, at about 300× g, at about 350× g, at about 400× g, at about 450× g, at about 500× g after the cell and medium is added to the plate.

In embodiments, the contents of the culture medium can be changed after culturing the ESC for a period of time. For example, the contents of the culture medium can be changed after about 8 hours, about 12 hours, about 16 hours, about 24 hours, about 36 hours, or about 48 hours after culturing the ESC. In embodiments, the medium can be replaced with a medium without the STAT3 agonist. In embodiments, the medium can be replaced with a medium without the SMAD2/3 agonist. In embodiments, the medium can be replaced with a medium without the Wnt agonist. In embodiments, the medium can be replaced with a medium without the inhibitor of the RAS-RAF MEK/ERK-MAPK pathway. In embodiments, the medium can be replaced with a medium without the fibroblast growth factor. In embodiments, the medium can be replaced with a medium without the PI3K-AKT agonist.

In embodiments, the ESC is cultured for a period of time sufficient to form the blastoid. In for example, the culturing can be about 1 day, about 2 days, about 3 days, about 4 days, about 5 days, about 6 days, about 7 days, about 8 days, about 9 days, about 10 days, or more as needed. In embodiments, the culturing can be about 1-5 days, about 2-6 days, about 3-7 days, about 4-8 days, about 5-9 days, or about 6-10 days.

In embodiments, the ESC can be cultured with one or more with additional cell types, such as those that facilitate production of the blastoid. For example, the ESC can be cultured with a population of a trophoblast stem cells, such as those described herein.

In embodiments, blastoids are derived from a mammalian ESC, as described herein. For example, the mammalian ESC can be from any mammal, such as a human, a primate, mouse, rat, dog, cat, bovine, cow, horse, pig, a fish, or a bird. For example, “bovine” can refer to an animal from the cattle group, non-limiting examples of which include cows, buffalo, and bison.

Aspects of the invention are further drawn towards methods of assisted reproduction of a subject. In embodiments, the method comprises obtaining or providing an ESC derived, isolated, or obtained from the subject. In embodiments, the method comprises culturing the ESC in a medium comprising one or more factors selected from the group consisting of a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof. In embodiments, the method comprises isolating a resulting blastoid. In embodiments, the method comprises transferring the resulting blastoid to a uterus.

In embodiments, the uterus of the subject is receptive to implantation. In embodiments, the subject can be treated with a medication in order to prepare the uterus for implantation. In embodiments, the menstrual cycle and endometrial thickness of the subject is monitored for receptivity to implantation.

In embodiments, the ESC is an induced ESC derived from a somatic cell. For example, the somatic cell can be any cell derived from a subject that is not a germ cell. Accordingly, any suitable somatic cell is contemplated to be used in methods herein, examples of which include, but are not limited to, an endothelial cell, an epithelial cell, a blood cell, an adipocyte, a neuron, an osteoclast, a chondrocyte, a myocyte, or other cell type.

Aspects of the invention can comprise a composition comprising an ESC and at least one factor selected from the group consisting of a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof.

Aspects of the invention provide a blastoid produced by a method described herein. Mammalian blastoids created using methods disclosed herein can be used for a variety of uses, including but not limited to including drug screening (such as determining drug toxicity), reproductive medicine, and other research uses and methods.

In embodiments, trophoblast cells and/or blastoids as described herein can be used in methods for determining drug toxicity. For example, the method can comprise (a) obtaining or providing a blastoid produced by a method according to any herein described method (b) contacting the trophoblast cell and/or blastoid as described herein with the drug; and (c) detecting signs of toxicity. Methods described herein encompass genetic manipulation of any of the populations of cells described herein. A genetic manipulation includes modifying, inserting, or deleting at least one of the genes in the cells.

Genetic manipulation may include transduction with a vector such as a nonintegrating vector (e.g., an episomal vector) or an integrating vector (e.g., lentiviral vector). In some embodiments, methods described herein involve genetically manipulating a population of cells using an episomal vector. Accordingly, in some embodiments, the population of cells involved in the methods described herein are gene-modified cells.

A “vector,” as used herein is any nucleic acid vehicle (DNA or RNA) capable of facilitating the transfer of a nucleic acid molecule into cells. In general, vectors include, but are not limited to, episomal vectors, plasmids, phagemids, viral vectors, and other vehicles derived from viral or bacterial sources that have been manipulated by the insertion or incorporation of a target nucleotide sequence. Viral vectors include, but are not limited to, vectors comprising nucleotide sequences derived from the genome of the following viruses: retrovirus; lentivirus; adenovirus; adeno-associated virus; SV 40-type viruses; polyomaviruses; Epstein-Barr viruses; papilloma viruses; herpes virus; vaccinia virus; polio virus. One can readily employ other vectors not named but known to the art.

Methods described herein encompass reprogramming the population of cells (e.g., the population of somatic cells) to a less differentiated state. Reprogramming, as used herein, refers to a process that alters or reverses the differentiation status of a cell (e.g., a somatic cell), which can be either partially or terminally differentiated. Reprogramming includes complete reversion, as well as partial reversion, of the differentiation status of a cell.

Aspects of the invention also provides for a kit for culturing, expanding, or growing a population of cells derived from a mammalian blastocyst. Non-limiting examples of components of the kit comprise cells, culture media, a vessel, and components as described herein, and instructions for use. The kit can be used to carry out the methods as described herein.

The cells can be packaged in the kit by any suitable means for transporting and storing cells. For example, the cells can be provided in frozen form, such as cryopreserved: dried form, such as lyophilized: or in liquid form, such as in a buffer. Cryopreserved cells, for example, can be viable after thawing.

A culture medium can can be included in the kit. For example, the culture medium can comprise essential and non-essential amino acids, vitamins, energy sources, lipids, and trace elements required by the cell for minimal growth and/or survival. The culture medium can comprise a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof. The culture medium can further comprise one or more factors selected from the group consisting of a ROCK kinase inhibitor, a pan-caspase inhibitor, an integrated stress response (ISR) inhibitor, and 1× polyamine supplement. The culture medium can be packaged by any suitable means for transporting and storing media.

The vessel can be a type of culture ware which provides a contamination barrier to protect the cultures from the external environment while maintaining the proper internal environment. For example, a vessel can be a flask, a tube, a Petri dish, a roller bottle, and/or a multi-well plate.

The instructions can include one or more of: a description of the cells of the kit; methods for thawing or preparing cells: culturing schedule: precautions; warnings; and/or references. The instructions can be printed directly on the container (when present), or as a label applied to the container, or as a separate sheet, pamphlet, card, or folder supplied in or with the container. A kit as described herein also includes packaging. In some embodiments, the kit includes a sterile container which contains a therapeutic or prophylactic composition: such containers can be boxes, ampules, bottles, vials, tubes, bags, pouches, blister-packs, or other suitable container forms known in the art. Such containers can be made of plastic, glass, laminated paper, metal foil, or other materials suitable for holding cells or medicaments.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Understanding the molecular mechanisms behind blastocyst formation and implantation is of critical importance for improving efficiency of assisted reproductive technologies. Recent advancements in in vitro embryo models derived from human and mouse stem cell cultures have opened up new avenues for understanding mechanistic insights into early lineage segregation and implantation. Here we have developed two strategies for the generation of blastocyst like structures (blastoids) from an ungulate species, bovine: i. 3D differentiation and self-organization directly from naïve-like bovine embryonic stem cells (bESCs), and ii. assembly through the combination of bovine trophectoderm stem cells (TSCs) and naïve-like ESCs. These structures have resembled bovine blastocysts in terms of morphology, size, cell number, and lineage composition and allocation. By testing medium conditions with different inhibition small molecules and cytokines, we have shown that the signaling including WNT, Hippo, MAPK, TGF-B and ERK are critical in the segregation of the bovine trophectoderm, epiblast and hypoblast lineages from naïve-like bovine ESCs.

### Example 2

Understanding the molecular mechanisms underlie blastocyst formation and implantation is of critical importance for improving the efficiency of assisted reproductive technologies (ARTs). Recent advancements in in vitro embryo models derived from human and mouse stem cell cultures have opened new avenues for understanding the mechanistic insights into early lineage segregation and implantation, and the development of promising ARTs toward improving animal reproduction efficiency. Here we have developed two strategies for the generation of blastocyst like structures (blastoids) from an ungulate species, Bos taurus: 1) 3D differentiation and self-organization directly from naïve-like bovine embryonic stem cells (bESCs), and 2) 3D assembly of bovine trophoblast stem cells (TSCs) and naïve-like bovine ESCs. These blastoids resemble bovine blastocysts in terms of morphology, size (blastoid: 333.3±35.42 μm, n=38, blastocyst: 274.87±0.68 μm, n=13), cell number, lineage composition and allocation as revealed by immunostaining of epiblast (Sox2), hypoblast (Sox17) and trophectoderm (CDX2, AP-2a, AP-2y, KRT18, ZOI) markers. In addition, extended culture of blastoids in in vitro growth media resulted in trophectoderm and cavity expansion up to ˜5 mm in diameter, which is comparable to IVF blastocysts controls. In order to evaluate the developmental competency, we transferred IVF produced blastocysts and blastoids into synchronized surrogates, and were able to detect the maternal recognition of pregnancy hormone Interferon-Tau (IFN-t) in the blood via ELISA in IVF embryos (78.36±21.54 μm/ml, n=2), assembled (56.53±25.13 μm/ml, n=2) and self-organized (74.47 μm/ml, n=1) blastoids. By testing different signaling pathways using small molecules and growth factors, we further show that WNT, Hippo, Ras-Raf-Mek-ERK/MAPK, Activin/TGF-B/SMAD pathways play important roles in the differentiation and segregation of the bovine trophectoderm, epiblast and hypoblast lineages from naïve-like bovine ESCs.

### Example 3

Continuing to accelerate the genetic improvement of livestock populations and improve their production efficiency are critical in our ability to meet the protein demands of a growing global population. Over the past half century, assisted reproductive technologies (ARTs) such as in vitro fertilization and somatic cell nuclear transfer have been used to improve reproductive efficiency of agricultural economic species; however, limitations remain, and better ARTs are warranted. Additionally, early embryonic mortality is a major cause of infertility in cattle, yet the molecular causes remain a mystery. We have established stem cell-based bovine blastocyst-like structures (blastoids), which resemble blastocysts in terms of morphology, size, cell number, and lineage composition and allocation. Bovine blastoids can be not only used for the development of new in vitro breeding program, but also great resources to study molecule mechanisms underlying early embryonic development. The goal of this project is to characterize developmental capacity of bovine blastoids and understand the molecular basis for cell-cell interactions towards embryonic development. Our long-term goal is to generate healthy pregnancies and calves from bovine blastoids. We have two objectives: 1) characterize developmental competence of bovine blastoids in vitro and in vivo; and 2) employ single-cell transcriptome profiling to characterize molecular phenotypes of cell lineage development of bovine blastoids. Completion of these objectives will lead to the development of innovative bovine blastoid technology for agricultural applications and expand our understanding of basic mechanisms regulating bovine embryonic development.

### Introduction

### Overall Goal and Specific Objectives

Continuing to accelerate the genetic improvement of livestock populations and improve their production efficiency are critical in our ability to meet the protein demands of a growing global population. Over the past half century, assisted reproductive technologies (ARTs) such as in vitro fertilization (IVF) has been widely used to improve reproduction efficiency of agricultural economic species. Additionally, somatic cell nuclear transfer (SCNT) has become an important ART that is fundamental to genetic manipulation of livestock, particularly for maximize utilizing the elite males and females. However, for distinct populations of elite animals, there are too few reproductively competent animals to maintain adequate genetic diversity, and challenges remain with the use of the ARTs for varies reasons. 1) very limited sources of functional reproductive cells (matured oocytes) for widely used ARTs. 2) low efficiency of directly clone animals by SCNT. Also, SCNT still relies on large number of oocytes. On the other hand, the greatest limitation to reproductive efficiency across mammalian species is embryonic mortality, estimated to be 25% to 45% or greater in ruminants. Most embryonic deaths occur during the first three weeks of pregnancy. Yet, the molecular mechanisms of this stage and how they go wrong remain a mystery.

A recent revolutionized technology that reconstitution of the blastocyst-like structures (blastoids) from pluripotent stem cells (PSCs) opens new avenues for the development of promising technologies toward improving animal reproductive efficiency and production. In our preliminary studies, we have developed two strategies for the generation of bovine blastoids: 1. 3D differentiation and self-organization directly from naive-like bovine embryonic stem cells (bESCs), and ii. assembly through the combination of bovine trophectoderm stem cells (TSCs) and naive-like ESCs (see preliminary data). The bovine blastoids have resembled bovine blastocysts in terms of morphology, size, cell number, and lineage composition and allocation. This has allowed us to create a new in vitro breeding (IVB) strategy to accelerate genetic diversity of elite populations in cattle, which includes 1) derivation of PSCs and/or TSCs from elite animals. 2) generation of blastoids. 3) transferring blastoids to surrogate to generate viable embryos and developing to term. The bovine blastoids technology will allow for a large paradigm shift in the reproductive field, especially for livestock reproduction.

Although IVB looks promising, our first challenge will be to develop bovine blastoids with an expanded ability to generate the full range of correct functioning embryonic and extra-embryonic tissues in vitro and in vivo. This will allow their elongation and attachment in utero, and eventually transform approaches for IVB. Our second challenge will be to utilize bovine blastoids platform to discover the molecular events that accompany blastocyst formation, embryo elongation and implantation. We will take advantage of these theoretically unlimited quantity of bovine stem cell-based embryo sources together with in vitro culture system to understand the molecule mechanisms underlie early embryonic loss. This example will validate these, i.e., characterize developmental capacity of bovine blastoids in vitro and in vivo, and understand the molecular basis for proper cell-cell interactions underlying bovine peri-implantation embryo development. Without wishing to be bound by theory, embodiments described herein can generate healthy pregnancies and calves from stem-cell derived blastoids.

### Objective 1. Characterize Developmental Competence of Bovine Blastoids In Vitro and In Vivo.

Without wihing to be bound by theory, bovine blastoids harbor the developmental capacity to generate viable embryos, similar to in vitro produced blastocysts. We will first validate whether the blastoids can give rise to embryonic and extraembryonic stem cells (ESC and TSC) using our established protocols. Second, we will determine whether bovine blastoids can self-organize into peri-implantation embryo-like structures in vitro. Third, a more stringent functional test for bovine blastoids is to determine whether they can enter elongation in utero. We will conduct initial assessment of in vivo developmental capacity of bovine blastoids.

### Objective 2. Employ Single-Cell Transcriptome Profiling to Characterize Molecular Phenotypes of Cell Lineage Development of Bovine Blastoids.

Our studies have established single cell atlas of bovine pre- and peri-implantation embryo development and characterized the molecular trajectories of lineage development during first three weeks of pregnancy. Objective 2 aims to conduct single cell RNA-sequencing analysis of blastoids and their derivative structures from Objective 1, and to determine transcriptional states of blastoid cell lineages by comparing with reference dataset from in vivo developed embryos.

Completion of these objectives will provide a thorough characterization and optimization of bovine stem cell-based blastocyst model. These innovations have the potential to reduce the use of mature gametes and produce essentially unlimited number of reproductive competent blastoids. The bovine blastoid technology can be incorporated to IVB strategy, which would allow substantial improvements in production efficiency in a short amount time and with a large reduction in the generation interval. In addition, completion of these objectives will also allow better understanding of embryonic loss and early pregnancies failure, thereby leading to advances in assisted reproductive technology.

The use of ARTs to improve animal reproductive efficiency in livestock species. Over the past half century. ARTs have been demonstrated their extraordinary ability to accelerate the genetic improvement of livestock populations and improve their productive efficiency. For example, in cattle, approximately one million in vitro produced embryos have been transferred in 2020, which accounted for 76.2% of all transferrable cattle embryos (both in vivo and in vitro produced) in 2020 worldwide (www.iets.org). For the last two decades, another technology, SCNT, has also been adapted into livestock industry and is fundamental to genetic manipulation of livestock, particularly for maximize utilizing the elite males and females. A common draw back of all current ARTs is their reliance on the use of mature oocytes, which are limited in quantity. This is particularly challenge for distinct elite animals or the highly endangered species. Thus, it is very important to develop innovative technologies to bypass the limitation of the current ARTs. On the other hand, the greatest limitation of IVF and SCNT is that the competence of embryos to establish a pregnancy is much lower than for embryos produced in utero. However, the physiological pathways that underlie embryonic deaths are not well understood. Therefore, a great understanding molecular mechanisms of bovine early embryonic development is warranted to further improve the embryo competence from ARTS.

Stem cell-based embryo models. Mammalian embryonic development starts with a fertilized egg, which cleavages to generate blastomeres. The blastomeres then compact, polarize, and undergo the first two lineage specification to generate three distinct cell types of a blastocyst. The first lineage segregation specifies an outer trophectoderm (TE) layer and an inner cell mass (ICM). ICM cells later segregate into epiblast (EPI) and primitive endoderm (PrE) lineages. In rodents, stem-cell lines from all three blastocyst lineages have been derived and stably maintained in vitro, namely embryonic stem cells (ESCs), trophoblast stem cells (TSCs), and extraembryonic endoderm stem cells (XENs). With all the necessary parts available, it is now possible to de novo assemble an artificial embryo by using cultured stem cells.

Early attempts to generate embryo-like structures (gastrulating embryos) are mostly from mouse, and more recent from humans. For example, the embryo-like structures can be generated from ESCs in 2D/3D differentiation cultures¹⁻³, or assembly of ESCs, TSCs, and/or XENs aggregates⁴⁻⁶. These embryo-like structures recapitulate several key morphogenetic characteristics of early post-implantation development, including lumenogenesis, epithelialization, and symmetry breaking to specify mesoderm and primordial germ-cell-like cells 4-6. These successes have stimulated the interest in recreating a pre-implantation blastocyst from PSCs. Most recently, a few groups have reported the success of generating both mouse⁷⁻⁹ and human¹⁰⁻¹³ blastocyst-like structures (blastoids) either through the sequential aggregation of PSCs and TSCs or through differentiation and self-organization of totipotent-like PSCs. In mouse, these blastoids morphologically resemble blastocysts and can implant and induce the formation of decidua upon embryo transfer⁷⁻⁹. The human blastoids also resemble human blastocysts in terms of their morphology, size, cell number, and composition and allocation of different cell lineages, and are amenable to embryonic and extra-embryonic stem cell derivation. Moreover, human blastoids can further develop into peri-implantation embryo-like structures in vitro and model several aspects of the early stage of implantation in vitro¹⁰.

Upon further optimization and generation of fully functional embryoids in vitro¹⁰⁻¹³, science is only a few steps away from tracing the full developmental path from cultured stem cells to viable offspring, of which significant progresses has been made in mice (personal communications at the annual scientific conference of International Society of Stem Cell Research, ISSCR). Undoubtedly, the discovery of mouse and human blastoids has set an invaluable precedent for reproductive sciences and fueled the enthusiasm of researchers and industries working on agricultural and endangered species to pursue similar approaches.

Establishment of bovine embryonic stem cells (bESCs). In recent years, there have been significant advances in identifying and stabilizing cultured bovine stem cells with prime or naïve pluripotency characteristics. Stable primed bESCs was reported using a custom TeSRl base medium supplemented with FGF2 and IWRl (CTFR medium)¹⁴. More recently. Zhao et al., reported the successful establishment of a bovine extend pluripotency (bEPSCs) from pre-implantation embryos using CTFRM supplemented with IWR-1/XAV939, WH-4-023, and ACTIVIN A¹⁵. The bEPSCs exhibit expanded developmental potential and have been shown to generate both embryonic and extraembryonic cell lineages. Efforts for establishing bovine iPSCs have also been reported using Yamanaka factors (OCT4, SOX2, KLF4, and MYC) or plus other transcriptional factors (KDM4A, LIN28, NANOG) in either retroviral, lentiviral, or PiggyBac vectors¹⁶⁻²³.

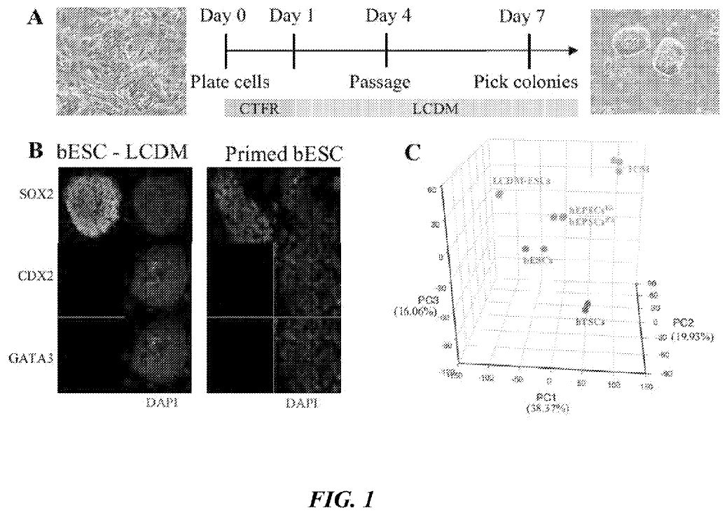

Further, we have generated and maintained both bESCs and bEPSCs following established protocols. Further, we have developed a new culture condition and allowed for the long-term culture of bESCs. In this condition, the bESCs are subsequently transited into naive-like ESCs (FIG. 1). With the recent significant processes of bovine stem cell research, cattle have been used as the pilot species in reproductive regenerative research and toward in vitro breeding²⁴

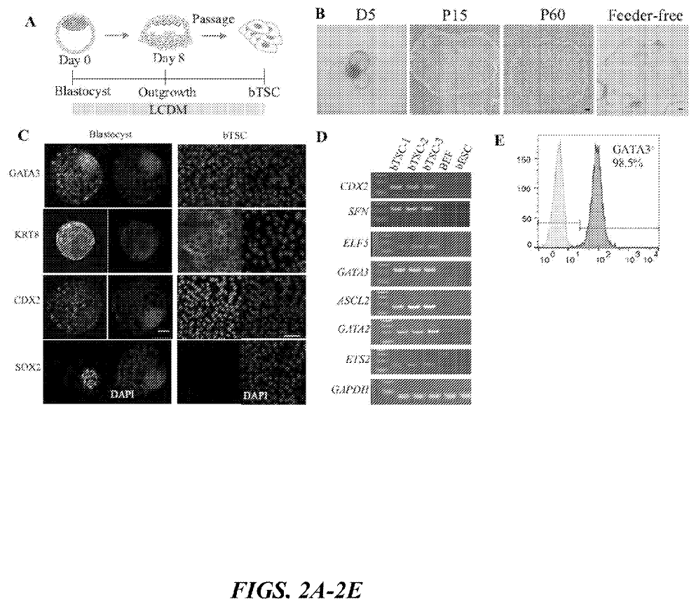

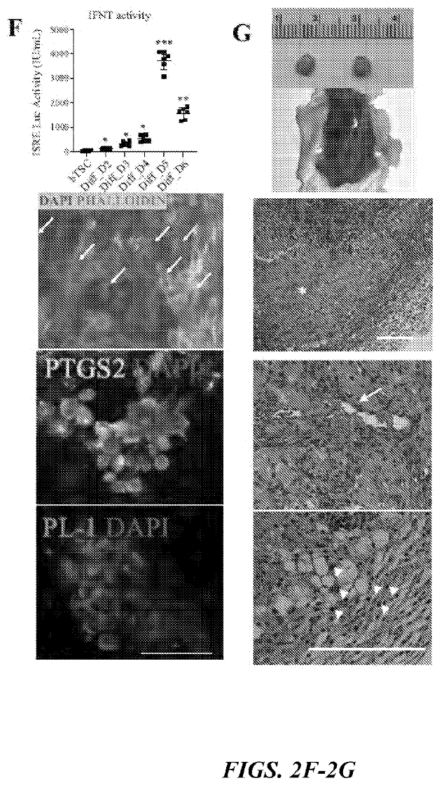

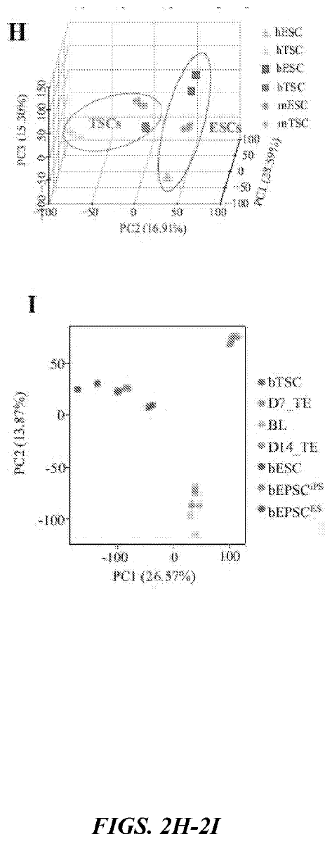

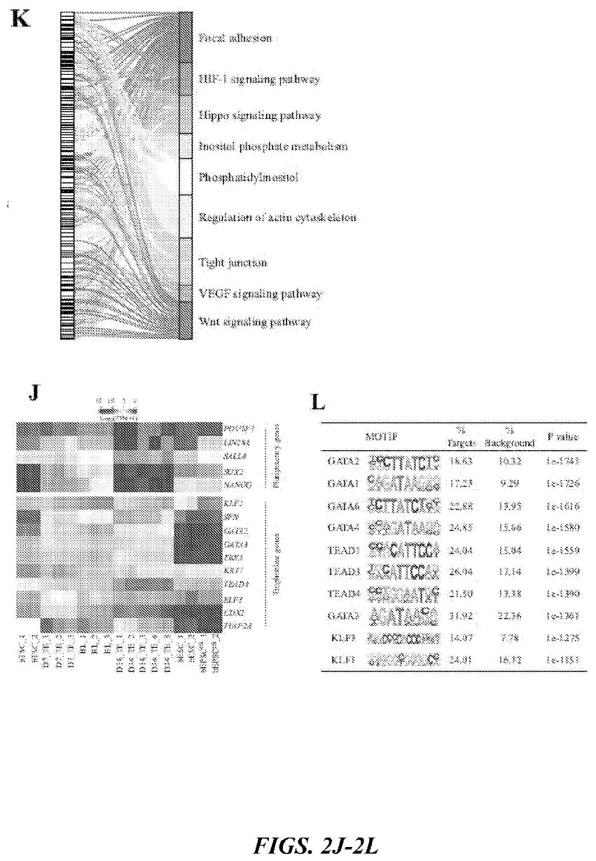

Derivation of bovine trophoblast stem cells (bTSCs) (Oral presentation at the 2022 Annual Conference of International Embryo Technologies Society, Savannah, manuscript in preparation). Placental trophoblast cells, arise from the TE of the blastocyst, are specialized cells in the placenta that mediate the interactions between the fetus and the mother. Trophoblast development and function are pivotal for the success of pregnancy. We have adapted a culture condition (LCDM), which was previously used for culturing mouse, human, pig and cow EPSCs (extended potential pluripotent stem cells), and surprisingly found that it could support robust derivation of bTSCs from blastocysts²⁵. bTSCs have maintained long-term colony morphology, mark gene expression, in vitro and in vivo developmental potential, and transcriptomic and epigenomic trophoblast lineage features (FIG. 2). The bovine TSCs we established not only provide a powerful model to study bovine early placental establishment and early pregnancy failure, but also contribute to the de novo assemble of blastocyst-like structures from cultured stem cells.

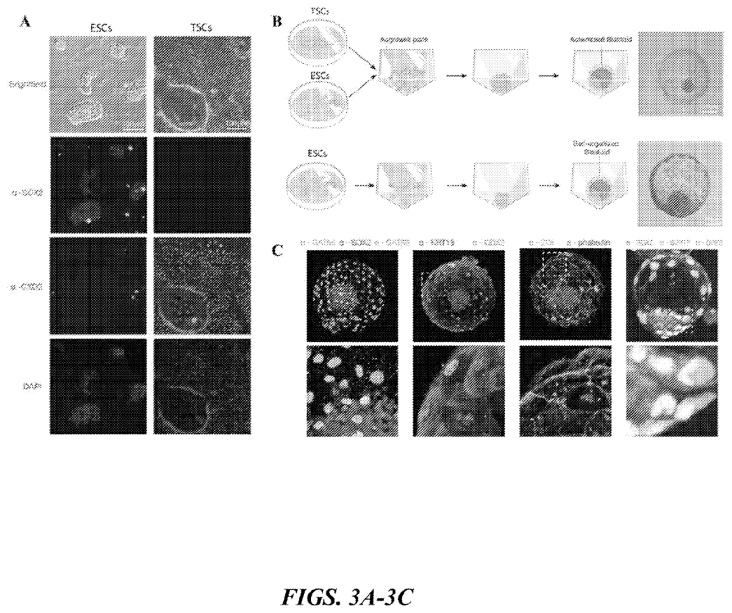

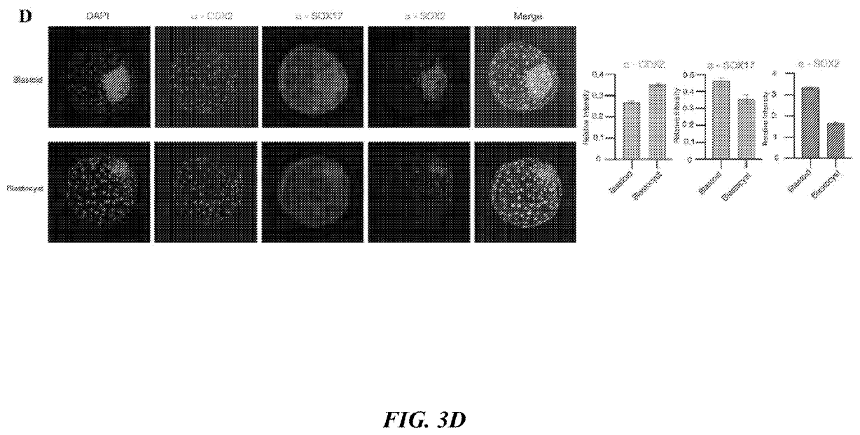

Establishment of stem cell-based bovine blastocyst-like structures (blastoids) (Poster presentation at the 2022 Annual Conference of Society for the Study of Reproduction. Spokane, manuscript in preparation). We have established two strategies for the generation of bovine blastoids. The first approach is through the assembly of bESCs and our recent established bTSCs (FIG. 3, panel A)²⁶. Briefly, a cell number ratio (16/16) of bESCs/bTSCs were placed in a 24 well of AggreWell culture plate with 1200 microwells per well (FIG. 3, panel B). The cells underwent three-step sequential culture condition (EDM and TDM3 are adapted from published human blastoids protocol 1²; LCDM was discovered that could support the derivation of stable bTSCs) (FIG. 2). The cells were cultured in a humidified incubator at 37° C. with 5% CO2 and 5% Oxygen. Everyday half of the media were replaced with fresh medium until day 10 where round and expanded blastocysts-like morphology are expected. With this approach, the bovine blastoids could be robust produced, and strikingly with a high efficiency (>80%). The second approach is through 3D differentiation and self-organization directly from bESCs. Briefly, a total of 16 bESCs were placed in the AggreWell and the cells underwent the same three-step sequential culture condition (FIG. 3, panel B). Similarly, bovine blastoids could be produced under this condition, but the efficiency is low due to the inefficient trophoblast differentiation from bESCs. Thus, we will focus on optimizing the conditions that bias the differentiation of EPSCs toward trophoblasts

In vitro breeding (/VB)—application of bovine blastoid technologies in livestock reproduction. There are two stem cell-based technologies with the potential for IVB. For the first, in vitro reconstitution of primordial germ cells (PGC) from PSCs has been succeed in multiple species including mouse²¹⁻³⁰, humans³¹, and rats³². The induced PGC-like cells (PGCLCs) can be used for IVB as they have the potential to develop into functional gametes, which can be fertilized and make in vitro embryos²⁴. However, it largely depends on advancements in the field of in vitro differentiation of PGCLCs, which are far behind for large animals. Generating functional gametes from induced PGCLCs remains challenging, even for rodents.

The second IVB strategy is to use the stem cell-based embryos as proposed here, which includes 1) derivation of PSCs and/or TSCs from elite animals, 2) generation of blastoids. 3) transferring blastoids to surrogate to generate viable embryos and developing to term. This IVB program, if successful, will provide several advantages over in vitro gametogenesis approach including much shortened generation period and potentially high efficiency.

### Rationale and Significance

Relationship to program area priorities. Developing innovative stem cell-based embryos to improve reproductive efficiency in cattle and understanding molecular basis of bovine embryogenesis make the proposed research directly relevant to the goal of the Animal Reproduction Program (A1211) of the USDA-AFRI FY 2022 to study embryonic and fetal development including interaction between the conceptus and its uterine environment.

Rationale and significance. The increase in the human population is expected to reach 9.8 billion by 2050³³ therefore, it is imperative that we identify methods to improve efficiency of animal reproduction and production that provides adequate, affordable, and high quality animal protein to consumers. Cattle have a key role in contributing to the global food supply as milk and meat³⁴. Therefore, bovine IVB strategy proposed in this study may be of significant help to address this issue, as the genetic gain achieved in a short time could be translated into more and better food to satisfy such demands.

The proposed IVB program for future cattle reproduction would begin with the somatic cells or embryos from elite animals. The next step would comprise the generation of large number of viable blastoids from cultured stem cells that derived from elite animals, which would be selected and transferred into recipient mothers and raised until puberty. First, the bovine blastoid technology could bypasses the use of mature eggs in current ARTs and produce essentially unlimited number of engineered blastocyst-like structures that have the potential to produce healthy pregnancies, therefore, it will become an integral part of breeding programs for a broad range of productive traits. Another advantage is its scalability, which allows to produce hundreds of thousands, if not re, blastoids within a short period of time. The molecule mechanisms underlie early embryonic loss remain largely unknown. Thus, these resources will allow us to decipher mechanisms into a time of development when most pregnancies fail and thereby lead to advances in ARTs. Furthermore, it will offer new screening routes for improving IVF culture conditions, pregnancy drug testing, environmental safety, and regenerative medicine. In addition, with blastoid technology, genotypes of distinct animals which would otherwise be lost due to failure to breed, injury or death could be reintroduced into the population as healthy young animals with significantly shortened generation intervals. For this purpose, the bovine makes it an ideal model to develop IVB technology for its application to other large animal species because the embryo manipulation technologies such as synchronization and embryo transfer, etc., that are essential for IVB program are well established in bovine than in any other mammalian species. Therefore, the findings from this study will also likely be applicable to other livestock further increasing the positive impact.

Innovation. The development of bovine blastoids from cultured stem cells for IVB is exciting and innovative in nature. It is conceivable that if normal pregnancies are produced from blastoids, this technology will allow for a large paradigm shift in the reproductive field, especially for livestock reproduction. Another innovative aspect of this proposal is the study of the cell-cell interactions underlying blastocyst formation and peri-implantation development, which would strengthen our understanding of bovine embryonic development. The study of the cell-cell interactions is impossible with bovine in vivo or IVF embryo settings. A final innovative aspect of our project is the use the state-of-the-art single cell RNA-sequencing, CRISPR-Cas9 mediated genetic perturbations of candidate receptors and ligands together with a readily accessible, scalable, versatile, and perturbable alternative cell model (blastoid) to IVF embryos, which will allow us to gain a more complete understanding of the mystery of bovine early development.

### Approach

Research approach. The foundation of our approach is that we have established bovine blastoids from cultured stem cells. In this proposal, we will first fully test the developmental potential of the bovine blastoids in vitro and in vivo. Second, we will employ single-cell transcriptome profiling to characterize molecular phenotypes of cell lineage development of bovine blastoids and their derivative structures. Finally, we will utilize blastoids as primary tools for identifying molecular mechanisms of cell-cell interactions controlling bovine pre- and peri-implantation development.

Stem cell lines: We have both female and male bESC and bTSC lines under active use in the laboratory. Although sex as variables will not be a concern in the development of blastoid technology as proposed in this study, we will consider using both male and female stem cell lines for sex effects if necessary. All lines are routinely tested for mycoplasma contamination before beginning a set of new experiments.

### Objective 1: Characterize Developmental Competence of Bovine Blastoids In Vitro and In Vivo

Overview: ESCs, XENs and TSCs, which are considered the in vitro counterparts of EPI, PrE and TE lineages, respectively, could be derived from blastocysts. In vivo, a viable bovine blastocyst will enter a critical two- to three-week periods of embryogenesis when the blastocyst undergoes extensive cellular proliferation and changes from a spherical shape to an elongated, filamentous form in preparation for implantation. Key events include pluripotency state transitions and the generation of epithelialized epiblast, followed by a symmetry-breaking event that leads to the formation of embryonic disc and subsequently gastrulation. Concomitantly, the extra-embryonic tissues undergo a global reorganization, with the hypoblast making the primary yolk sac and the trophoblast differentiating into the major cell types of the placenta. These morphogenetic transformations need to be tightly coordinated. Failure of development during this time represents one of the major causes of early pregnancy loss in bovine.

We have generated bovine blastoids that mimic blastocysts in terms of morphology, size, cell number, and marker gene expression (FIG. 3). Before they can be incorporated into IVB program, the developmental competence of bovine blastoids needs to be evaluated to recapitulate abovementioned critical developmental landmarks. Therefore, the primary goal of Objective 1 is to characterize whether bovine blastoids can recapitulate bovine embryo development in vitro and in vivo. We will first test if the blastoids have the capability to give rise to the major stem cell types (ESCs and TSCs) that can be derived from a pre-implantation blastocyst using our established culture conditions (FIG. 1 and FIG. 2).

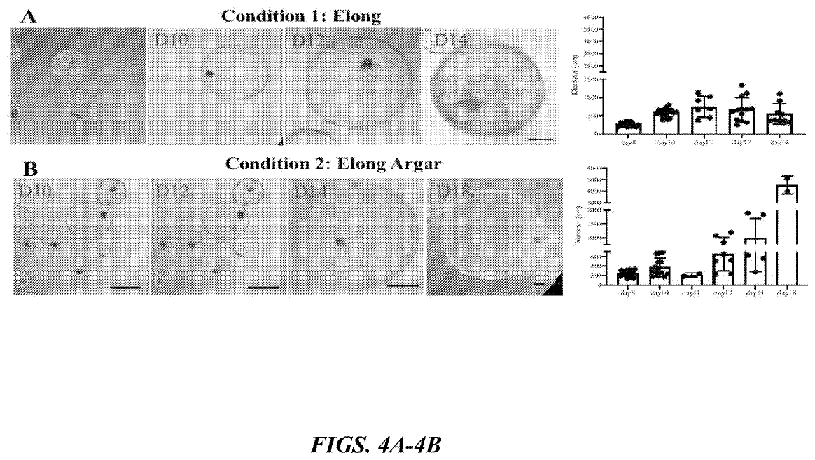

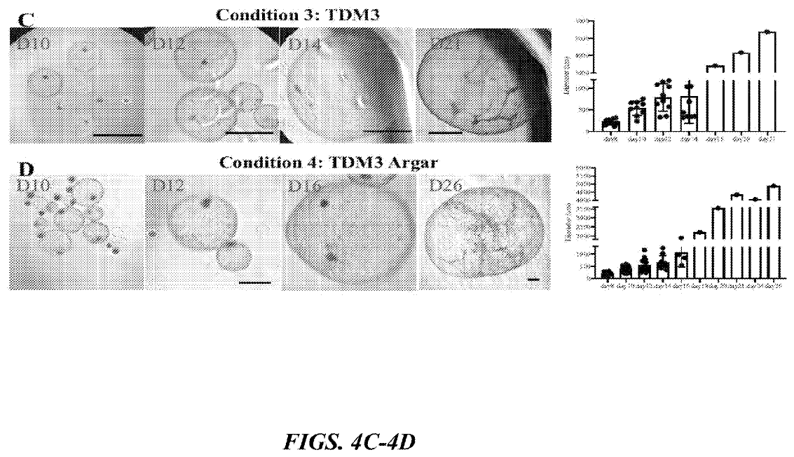

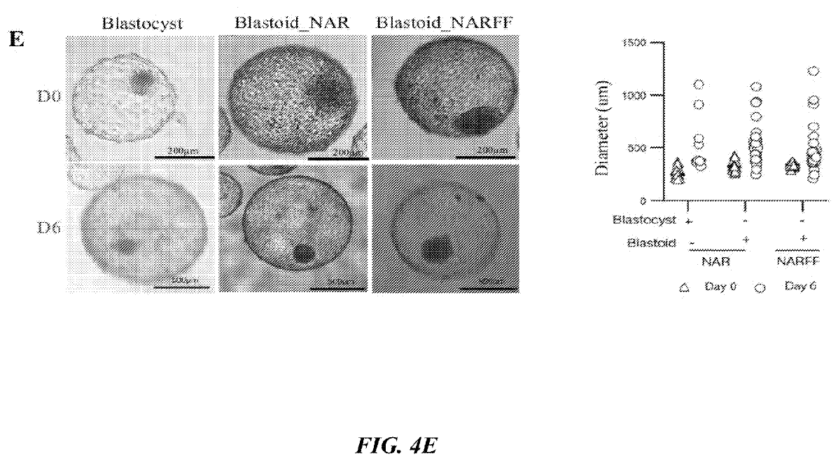

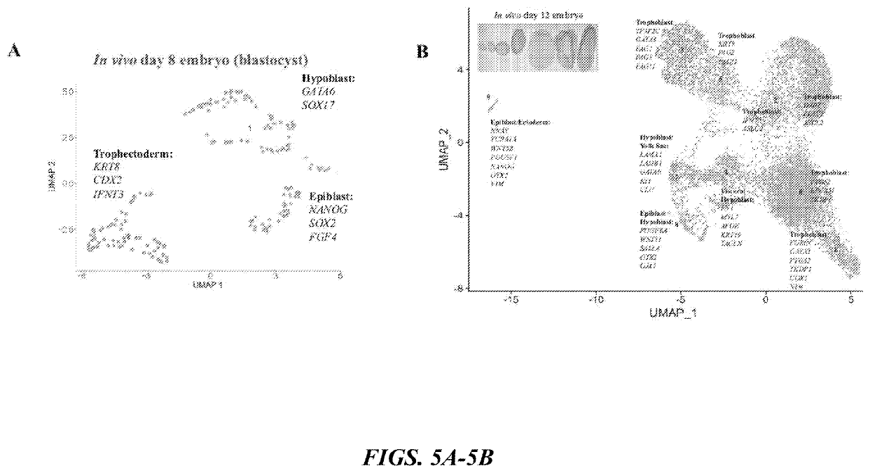

Secondly, we will determine whether bovine blastoids can self-organize into structures similar to peri-implantation embryos. In our preliminary studies, we have established bovine blastocyst in vitro growth system by following published protocols based on a elongation condition (Elong: Neurobasal and DMEM/F12 supplemented with IX Glutmax, IX NEAA, IX N2, IX B27, 20 ng/ml Activin A and 10 uM ROCK inhibitor), with one supports bovine trophectoderm proliferation, hypoblast migration and epiblast survival after the blastocyst stage³⁵, the other allows day 14 in vitro sheep embryos to recapitulate most developmental landmarks of in vivo embryos during the second week of development, including the initiation of gastrulation 36. We have further optimized the in vitro culture conditions (FIG. 4, panel A, panel B: Elong medium with or without Argarose coated, and FIG. 4, panel C, panel D: a new condition TDM3 with or without Argarose) and established a robust bovine blastocyst in vitro growth system. In addition, we have had initial success for the in vitro growth of bovine blastoids under a N2B27 supplement with small molecules (activinA, ROCK.i, bFGF and IGFI) (named as NAR or NARFF) that could promote epiblast survival and pluripotency (FIG. 4, panel E).

Derivation of ESCS/EPSC's and TSCs from bovine blastoids—By developing culture conditions of CTFR14, CTFRM15, and LCDM25, we have successfully derived bovine primed ESCs. EPSCs. and TSCs from individually plated IVF blastocysts, respectively. Under the same conditions, we will re-derive the ESCs/EPSCs and TSCs from bovine blastoids. The derived cell lines will be assessed in terms of self-renewal, marker gene expression and immunostaining analysis, in vitro differentiation, and in vivo teratoma assays as we previously published 14-25. The goal of these experiments is to demonstrate whether bovine blastoids are amenable to stem cell derivation from blastocyst lineages, and that these blastoid-derived stem cells can further differentiate into respective downstream cell types.

In vitro growth of bovine blastoids—To evaluate whether bovine blastoids could recapitulate some aspects of bovine early development in vitro, we will test following conditions with or without agarose coated plates based on our preliminary studies (FIG. 4).

Elong:1:1 Neurobasal and DMEM/F12 basal medium supplemented 20 ng/ml Activin A and 10 μM ROCK inhibitor]36

TDM3: N2B27 basal medium supplemented with 1 uM PD0325901 (inhibitor of MEK1 and MEK2), 1 μM A83-01 (inhibitor of TGF-b). 0.5 μM SB590885 (inhibitor of B-raf), 1 μMWH-4-023 (Lek and Src inhibitor), 0.5 μM IM-12 (activator of Wnt), 1 μM CHIR99021 (activator of Wnt). 10 ng/mL LIF and 0.2 mM VPA.

ElongF: Elong medium supplement with FGF4.

For the preparation of agarose coated plates, 2.4% ultrapure low melting point agarose will be prepared in PBS and autoclaved, then poured into the wells when cold down to around 45° C. The plate will be placed on ice for rapid solidification of the gel. The medium will be poured on the gel and incubated at 38.5° C. 5% CO2. The medium will be changed every day until use.

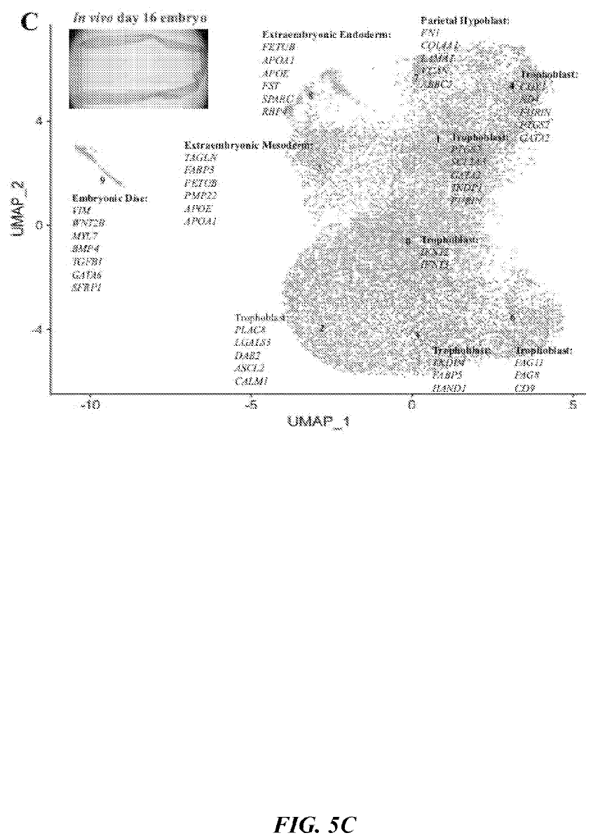

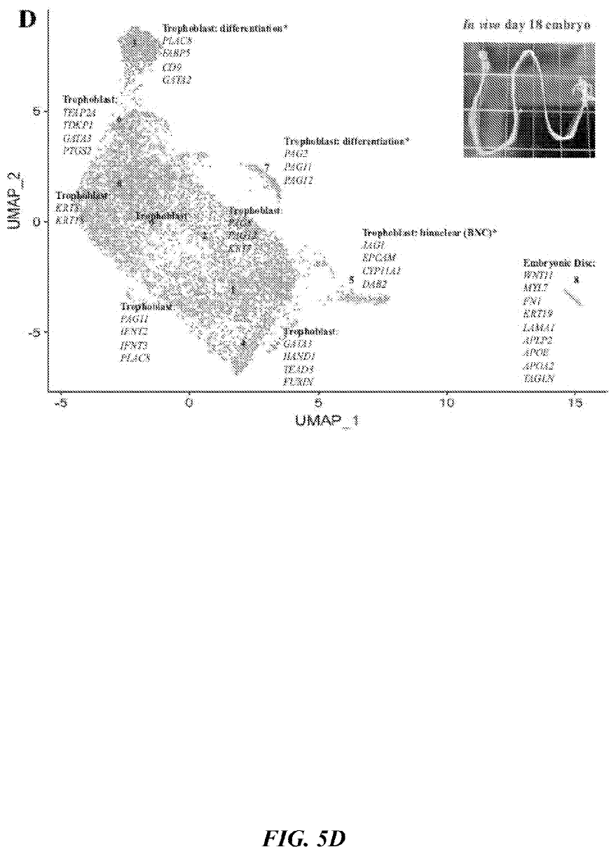

Surviving bovine blastoids will be expected to develop in a spherical shape. The diameter, area, and volume will be measured individually following one month of in vitro culture. Immunostaining analysis will be performed using the antibodies SOX2 for epiblast. SOX17 for hypoblast. CDX2 for trophectoderm cells as described35. Gene expression analysis will include trophoblast differentiation markers such as IFNT2 (interferon tau 2, produced by mononuclear trophoblast cells and the principle embryonic signal for pregnancy recognition in ruminants39,40), and PL-I (placental lactogen 1, a binuclear trophoblast marker and plays a vital role in placentation41). The culture medium will be collected at different time points to examine IFNT activity using a Luciferase-based IFN stimulatory response element (ISRE) assay during differentiation42. The in vitro growth of IVF blastocysts will be used as control. Marker gene expression measurements will be compared to in vitro cultures of IVF blastocyst as well as the gold standard in vivo embryos (day 8 to day 18) (see FIG. 5).

In vivo developmental potential of bovine blastoids—We have had success with transferring 15-20 blastocysts to a single recipient and recovery of elongated embryos by flushing one week later for analysis43. Using the same system, high quality of bovine blastoids derived from two strategies and IVF blastocyst controls will be transferred to recipients (20 blastoids or blastocysts per recipient) and recovered around day 12 to day 18. The flushing bovine embryos at different peri-implantation stages (from day 12 to day 18) is a routine procedure in the PI laboratory (see FIG. 5).

We will examine whether morphologically elongated structures can be obtained as an indication of viability. Following collection, the derived structures will be exanimated for the embryonic disc and lumen formation. The structures will then be fixed for immunostaining analysis of different cell lineages. The blood samples from the recipients will be collected to perform pregnancy test using a bovine Interferon-Tau ELISA Kit (Lifeome BioLabs Inc). Morphological and marker gene expression measurements will also be compared to the gold standard in vivo embryos (day 12 to day 18) collected by flushing (FIG. 5). We anticipate performing ten rounds of transfers/flushing (20 cows for blastoids generated from two approaches and 10 cows for IVF blastocysts each round).

Statistics. Each experiment will be replicated at least three occasions except as otherwise stated. For quantitative data, statistical analyses will be performed by using GraphPad Prism 5 software (GraphPad Software, Inc., CA). Two-way comparisons performed at a single time-point will be made with a Student's t test. Data followed over time will be subjected to one-way ANOVA followed by Tukey's test. Comparisons made on treatment effects over time will be analyzed by two-way ANOVA. Values of P:S 0.05 will be considered to support the conclusion that differences are significant.

Anticipated outcomes, potential pitfalls, and alternative strategies. The proposed experiments will provide a comprehensive characterization of the developmental potential of the established bovine blastoids. The major outcome of the research will be to determine the viability of bovine blastoids and provide a proof of concept for their suitability for the IVB program. The proposed experiments are based on our extensive preliminary data that bovine blastoids mimic blastocysts in terms of morphology, size, cell number, and marker gene expression (FIG. 3). The PI laboratory has also established a robust in vitro growth system for bovine IVF blastocysts, which sets a foundation for the proposed bovine blastoid in vitro growth. In addition, both PI and Co-Pl laboratories have extensive experience in the characterization of bovine ESCs and TSCs and in vitro embryo cultures, and Pl's laboratory is well-versed in bovine embryo transfer/flushing. Therefore, we do not anticipate any significant technical challenges.

However, we recognize that our proposed in vitro and in vivo blastoid experiments can only model certain aspects of bovine early embryo development, therefore, future studies are needed to follow the development of transferred blastoid for a longer period and to determine whether full-term pregnancy can be achieved. Also, in our in vitro growth system, although the blastocysts can rapidly grow, they are still not able to elongate perfectly like in vivo situations. We recognize that we are still far away from the long-term and functional sustain of bovine embryos in vitro due to the extensive changes from a spherical shape to an elongated, filamentous form of bovine embryo in nature, further optimization will likely be needed. Nevertheless, the use of parallel strategies for in vitro development, as well as our gold standard in vivo developmental test mitigates risk associated with Objective 1. Another potential pitfall is that immunostaining analysis is not sufficient to identify and compare all cell lineages of elongated embryos derived from blastoids and blastocysts. However, we will perform single cell RNA-sequencing (10) Genomics) of elongated embryos from both conditions and compare with their in vivo counterparts, which will allow us to dissect the similarity and difference in terms of lineage composition and their gene expression between the two as proposed in Objective 2.

### Objective 2. Employ Single-Cell Transcriptome Profiling to Characterize Molecular Phenotypes of Cell Lineage Development of Bovine Blastoids.

Overview. Embryos produced in vitro, although have capacity to develop to term, differ in biochemical, molecular, and ultrastructural characteristics from those developing in vivo 44. Therefore, it is essential to uncover the similarity and difference of molecular phenotypes between the blastoids and their in vitro derivative structures and their counterparts developed in vivo. In our preliminary studies, we have established a single cell atlas of bovine pre-implantation and peri-implantation embryo development (FIG. 5). Using single cell RNA sequencing (scRNA-seq) analysis, we mapped the transcriptome trajectories of different cell lineages in in vivo developed bovine embryos from day 8 blastocysts to day 18 elongating embryos. In blastocyst, we have captured all three lineages including epiblast, hypoblast, and trophectoderm cells of a pre-implantation blastocyst (FIG. 5, panel A). The embryos underwent significant elongation in size in day 12, 16, and 18 elongated embryos (FIG. 5, panels B, C and D). We were able to capture the molecular trajectories of cell lineages during peri-implantation development (from day 8 blastocyst to day 18 elongated embryo) (FIG. 5). This single cell atlas will serve as a gold standard reference for the assessment of cell identities and molecular characteristics of lineage development in bovine blastoids and their derivative structures following in vitro and in vivo development. It will also provide new insights into further optimization of the bovine blastoid methods.

In the Objective 2, using our established scRNA-seq analysis pipeline, we will first characterize bovine blastoids that mimic blastocysts with respects to the molecular phenotypes of their cell lineages. Second, as shown in FIG. 4, without wishing to be bound by theory, bovine blastoids will recapitulate some aspects of early development in vitro. Therefore, we will further determine the transcriptomic features of bovine blastoid development in vitro and in vivo using scRNA-seq and compare them with reference dataset from in vivo-derived embryos.

Cell isolation. Bovine blastoids (self-organized and assembly) cultured in 24 well of AggreWell culture plate with 1200 microwells per well (28,800 individual cultures) will be collected and digested with a solution containing Dispase II (1.25 U/ml), collagenase N (0.4 mg/ml, Sigma-Aldrich), and DNasel (80 U/ml) in DPBS for 30 min at 37° C. Single cell suspension will be prepared using the 10× Genomics Cell Preparation protocol (Pleasanton, CA). Approximately 2×10⁵ cells will be washed and passed through a 40 μm cell strainer. In the Jiang laboratory, cell viability ranges between 90-95%. Approximately 5×10⁴ embryonic cells are targeted for single cell analysis.

scRNA-seq library preparation. Single cells will be captured by the Chromium Controller into 10× barcoded gel beads. Single cell cDNA libraries will be prepared using Chromium Single Cell 3′ V3 Reagent Kit and Chromium Controller (10× Genomics), followed by quality control at the UF Genome Facility (see resources). Libraries from blastoids and blastocysts will be processed by multiplexing and sequencing using the Illumina NovaSeq 6000 Sequencing System. We will process three libraries per experimental condition (three replicates for each of self-orgainzed/assembled blastoids). It is expected to profile at least 10,000 cells per sample and generate at least 25,000 reads per cell, allowing identification of all cell lineages using an adjusted P-value≤0.05.

scRNA-seq data analysis. scRNA-seq raw sequencing reads will be processed using the CellRanger count pipeline (v6.1.2. 10× Genomics) with default parameters and trimmed sequencing reads will be aligned to the bosTau9_UCSC bovine reference genome. We will use widely adopted standards from the community to identify high quality cells, remove mitochondrial-enriched cells, and remove cell doublets. Standard quality control and pre-processing of count files (including data normalization), and identification of cell clusters will be carried out using the Seurat toolkit (v4) and according to t-SNE/UMAP clusters generated by Loupe Cell Browser (10× Genomics). Cell clusters will be assigned identities based on known marker gene expression (e.g., SOX2 and OCT4 for epiblast, SOX17 and GATA6 for hypoblast, CDX2, GATA3 and TFAP2A for trophectoderm cells), as well as based on gene ontology enrichment analysis and cell-specific gene enrichment analysis. Top expressed genes identified in each cluster will be used for pathway analysis using Ingenuity Pathway Analysis (IPA) software (Qiagen) to further characterize function of specific lineages. Two separate analyses will be performed to compare the blastoids and blastocysts. First analysis of variance will be performed to determine whether the lineage compositions vary between blastoids and blastocysts. Secondly, gene expression profiles for individual cell lineages will be determined based on an FDR-corrected Fisher's exact test of P-value<0.05 and log 2 fold change>1.

Single cell atlas to uncover cell lineage identities of bovine blastoids in the establishment of elongation. We anticipate that bovine blastoids could recapitulate some aspects of early development in vitro and in vivo as proposed in Objective 1. Therefore, we will conduct scRNA-seq analysis to define the identity of the derived structures and determine the transcriptional states of bovine blastoids in vitro and in vivo derivates by comparing the gold standard reference of in vivo embryos. The subsequent appearance of cell lineages as shown (FIG. 5) in the normal embryo development will be our focus in the analysis of blastoid derived structures.

Statistics. Besides of the abovementioned statistic test, an issue for scRNA-seq experiments is the power. However, typically scRNA-seq experiments do not need replicates. Additionally, more blastoids and blastoids cultures can be produced easily if needed.

Anticipated outcomes, potential pitfalls, and alternative strategies. The scRNA-seq methodology and bioinformatics analyses pipeline proposed in this objective are all established in both PI and Co-PI laboratories. Jiang laboratory has most recently performed scRNA-seq experiments on the bovine in vivo derived embryos from pre-implantation blastocyst to peri-implantation embryo at days 12, 16, and 18 (FIG. 5). We anticipate the number of cells in specific cell lineages (EPVPE/TE) will be similar between blastoids and blastocysts. We also expect to identify the similarities and differences of gene expression patterns between blastoids and blastocysts. The subsequent appearance of cell lineages in the blastoid in vitro and in vivo derived structures that similar as normal embryo development as shown in FIG. 5 will also provide a molecular prospect of the viability of blastoids. Single cell transcriptome profiling will help elucidate cellular dynamics and hierarchical regulation during the development of the blastoids.

In the Objective 2, the scRNA-seq dataset of in vivo development bovine embryos were generated from pooled embryos (pooled blastocysts or day 12 elongated embryos to get enough cell for scRNA-seq analysis) without the separation of sex. The blastoids pooled for scRNAseq analysis can be generated from both male and female ESC and TSC lines. Because our focus will be comparing cell identities and lineage differentiation dynamics, therefore, sex as a biology variable will likely not be a major contributing factor. To address this potential pitfall, if needed, we will computationally separate male and female cells from the reference dataset and used for comparison with male and female bastoids. Another limitation is lack of spatial information in the scRNA-seq. We will consider performing single cell spatial transcriptome analysis (10× Visium Spatial Gene Expression) if budget allows. Alternatively, in situ hybridization will be used as a complementary approach to positionally identify differentially regulated transcripts between blastoids and their derived in vitro cultures and in vivo matched stage of embryo.

### BIBLIOGRAPHY & REFERENCES CITED IN THIS EXAMPLE

1. Poh, Y. C. et al. Generation of organized germ layers from a single mouse embryonic stem cell. Nat Commun 5, 4000, doi: 10.1038/ncomms5000 (2014).
2. Warmflash, A., Sorre, B., Etoc. F., Siggia. E. D. & Brivanlou. A. H. A method to recapitulate early embryonic spatial patterning in human embryonic stem cells. Nat Methods 11, 847-854, doi: 10.1038/nmeth.3016 (2014).
3. Beccari. L. et al. Multi-axial self-organization properties of mouse embryonic stem cells into gastruloids. Nature 562, 272-276, doi: 10.1038/s41586-018-0578-0 (2018).
4. Harrison. S. E., Sozen. B., Christodoulou, N., Kyprianou, C. & Zernicka-Goetz, M. Assembly of embryonic and extraembryonk stem cells to mimic embryogenesis in vitro. Science 356, doi: 10.1126/science.aall810 (2017).
5. Sozen. B. et al. Self-assembly of embryonic and two extra-embryonic stem cell types into gastrulating embryo-like structures. Nat Cell Biol 20, 979-989, doi: 10.1038/s41556-018-0147-7 (2018).
6. Zhang, S. et al. Implantation initiation of self-assembled embryo-like structures generated using three types of mouse blastocyst-derived stem cells. Nat Commun 10. 496. doi: 10.1038/s41467-019-08378-9 (2019).
7. Rivron, N. C. et al. Blastocyst-like structures generated solely from stem cells. Nature 557, 106-111, doi: 10.1038/s41586-018-0051-0 (2018).
8. Li, R. et al. Generation of Blastocyst-like Structures from Mouse Embryonic and Adult Cell Cultures. Cell 179, 687-702 e618, doi: 10.1016/j.cell.2019.09.029 (2019).
9. Sozen, B. et al. Self-Organization of Mouse Stem Cells into an Extended Potential Blastoid. Dev Cell 51, 698-712 e698, doi: 10.1016/j.devcel.2019.11.014 (2019).
10. Liu, X. et al. Modelling human blastocysts by reprogramming fibroblasts into iBlastoids. Nature 591, 627-632. doi: 10.1038/s41586-021-03372-y (2021).
11. Yanagida, A. et al. Naive stem cell blastocyst model captures human embryo lineage segregation. Cell Stem Cell 28, 1016-1022 el 014, doi: 10.1016/j.stem.2021.04.031 (2021).
12. Yu, L. et al. Blastocyst-like structures generated from human pluripotent stem cells. Nature 591, 620-626, doi: 10.1038/s41586-021-03356-y (2021).
13. Kagawa, H. et al. Human blastoids model blastocyst development and implantation. Nature 601, 600-605, doi: 10.1038/s41586-021-04267-8 (2022).
14. Bogliotti, Y. S. et al. Efficient derivation of stable primed pluripotent embryonic stem cells from bovine blastocysts. Proc Natl Acad Sci USA 115, 2090-2095, doi: 10.1073/pnas. 1716161115 (2018).
15. Zhao, L. et al. Establishment of bovine expanded potential stem cells. Proc Natl Acad Sci USA 118. doi: 10.1073/pnas.2018505118 (2021).
16. Han, X. et al. Generation of induced pluripotent stem cells from bovine embryonic fibroblast cells. Cell Res 21, 1509-1512. doi: 10.1038/cr.2011.125 (2011).
17. Sumer, H. et al. NANOG is a key factor for induction of pluripotency in bovine adult fibroblasts. J Anim Sci 89, 2708-2716. doi: 10.2527/jas.2010-3666 (2011).
18. Deng, Y. et al. Generation of induced pluripotent stem cells from buffalo (Bubalus bubalis) fetal fibroblasts with buffalo defined factors. Stem Cells Dev 21, 2485-2494, doi: 10.1089/scd.2012.0018 (2012).
19. Kawaguchi, T. et al. Generation of Naive Bovine Induced Pluripotent Stem Cells Using PiggyBac Transposition of Doxycycline-Inducible Transcription Factors. PLOS One 10, e0135403. doi: 10.1371/journal.pone.0135403 (2015).
20. Talluri, T. R. et al. Derivation and characterization of bovine induced pluripotent stem cells by transposon-mediated reprogramming. Cell Reprogram 17, 131-140, doi: 10.1089/cell.2014.0080 (2015).
21. Malaver-Ortega, L. F., Sumer, H., Liu, J. & Verma, P. J. Inhibition of JAK-STAT ERK/MAPK and Glycogen Synthase Kinase-3 Induces a Change in Gene Expression Profile of Bovine Induced Pluripotent Stem Cells. Stem Cells Int 2016, 5127984. doi: 10.1155/2016/5127984 (2016).
22. Zhao, L. et al. Characterization of the single-cell derived bovine induced pluripotent stem cells. Tissue Cell 49, 521-527, doi: 10.1016/j.tice.2017.05.005 (2017).
23. Su, Y. et al. Establishment of Bovine-Induced Pluripotent Stem Cells. Int J Mo/Sci 22, doi: 10.3390/ijms221910489 (2021).
24. Goszczynski, D. E. et al. In vitro breeding: application of embryonic stem cells to animal production dagger. Biol Reprod 100, 885-895, doi: 10.1093/biolre/ioy256 (2019).
25. Wang, Y. et al. 3 Derivation of bovine trophoblast stem cells. Reproduction, Fertility and Development 34, 235-235, doi: https://doi.org/10.1071/RDv34n2Ab3 (2022).
26. Wang, Y. et al. 3& #x2003: Derivation of bovine trophoblast stem cells. Reprod Fertil Dev 34,235, doi: 10.1071/RDv34n2Ab3 (2021).
27. Hayashi, K. et al. Offspring from oocytes derived from in vitro primordial germ cell-like cells in mice. Science 338, 971-975, doi: 10.1 126/science. 1226889 (2012).
28. Hayashi, K., Ohta, H., Kurimoto. K., Aramaki, S. & Saitou. M. Reconstitution of the mouse germ cell specification pathway in culture by pluripotent stem cells. Cell 146, 519-532, doi: 10.1016/j.cell.2011.06.052 (2011).
29. Hikabe, O. et al. Reconstitution in vitro of the entire cycle of the mouse female germ line. Nature 539, 299-303, doi: 10.1038/nature20104 (2016).
30. Ishikura, Y. et al. In Vitro Derivation and Propagation of Spermatogonial Stem Cell Activity from Mouse Pluripotent Stem Cells. Cell Rep 17, 2789-2804. doi: 10.1016/j.celrep.2016.11.026 (2016).
31. Yamashiro, C. et al. Generation of human oogonia from induced pluripotent stem cells in vitro. Science 362, 356-360, doi: 10.1126/science.aat1674 (2018).
32. Oikawa, M. et al. Functional primordial germ cell-like cells from pluripotent stem cells in rats. Science 376, 176-179, doi: 10.1126/science.abl4412 (2022).
33. Nations, U. World Population Prospects: The 2017 Revision. (2017).
34. FAO, F. a. A. 0. o. t. U. N. More Fuel for the Food/Feed Debate. (2018).
35. Ramos-Ibeas, P. et al. Embryonic disc formation following post-hatching bovine embryo development in vitro. Reproduction 160, 579-589, doi: 10.1530/REP-20-0243 (2020).
36. Ramos-Ibeas, P. et al. In vitro culture of ovine embryos up to early gastrulating stages. Development 149, doi: 10.1242/dev.199743 (2022).
37. Spencer, T. E., Ott, T. L. & Bazer, F. W. tau-Interferon: pregnancy recognition signal in ruminants. Proc Soc Exp Biol Med 213, 215-229, doi: 10.3181/00379727-213-44053 (1996).
38. Roberts, R. M., Ealy, A. D., Alexenko, A. P., Han, C. S. & Ezashi, T. Trophoblast interferons. Placenta 20, 259-264, doi: 10.1053/plac.1998.0381 (1999).
39. Mamo, S. et al. RNA sequencing reveals novel gene clusters in bovine conceptuses associated with maternal recognition of pregnancy and implantation. Biology of reproduction 85, 1143-1151 (2011).
40. Bazer, F. W., Wu, G. & Johnson, G. A. Pregnancy recognition signals in mammals: the roles of interferons and estrogens. Animal Reproduction (AR) 14, 7-29 (2018).
41. Nakaya. Y., Kizaki, K., Takahashi, T., Patel, O. V. & Hashizume. K. The characterization of DNA methylation-mediated regulation of bovine placental lactogen and bovine prolactin-related protein-I genes. BMC Molecular Biology 10, 1-14 (2009).
42. McCoski, S. R. et al. Validation of an interferon stimulatory response element reporter gene assay for quantifying type I interferons. Domest Anim Endocrinol 47, 22-26. doi: 10.1016/j.domaniend.2013.12.003 (2014).
43. Gutierrez-Castillo, E. et al. Effect of vitrification on global gene expression dynamics of bovine elongating embryos. Reprod Fertil Dev 33, 338-348. doi: 10.1071/RD20285 (2021).
44. Hansen, P. J. Implications of Assisted Reproductive Technologies for Pregnancy Outcomes in Mammals. Annu Rev Anim Biosci 8, 395-413, doi: 10.1146/annurev-animal-021419-084010 (2020).
45. Zhu, M. & Zernicka-Goetz. M. Principles of Self-Organization of the Mammalian Embryo. Cell 183, 1467-1478, doi: 10.1016/j.cell.2020.11.003 (2020).
46. Feldman, B., Poueymirou, W., Papaioannou, V. E., DeChiara, T. M. & Goldfarb, M. Requirement of FGF-4 for postimplantation mouse development. Science 267, 246-249, doi: 10.1126/science.7809630 (1995).
47. Arman, E., Haffner-Krausz, R., Chen, Y., Heath, J. K. & Lonai, P. Targeted disruption of fibroblast growth factor (FGF) receptor 2 suggests a role for FGF signaling in pregastrulation mammalian development. Proc Natl Acad Sci USA 95, 5082-5087, doi: 10.1073/pnas.95.9.5082 (1998).
48. Cheng, A. M. et al. Mammalian Grb2 regulates multiple steps in embryonic development and malignant transformation. Cell 95, 793-803, doi: 10.1016/s0092-8674 (00) 81702-x (1998).
49. Chazaud, C., Yamanaka. Y., Pawson, T. & Rossant, J. Early lineage segregation between epiblast and primitive endoderm in mouse blastocysts through the Grb2-MAPK pathway. Dev Cell 10, 615-624, doi: 10.1016/j.devcel.2006.02.020 (2006).
50. Schrode, N., Saiz, N., Di Talia, S. & Hadjantonakis, A. K. GATA6 levels modulate primitive endoderm cell fate choice and timing in the mouse blastocyst. Dev Cell 29, 454-467, doi: 10.1016/j.devcel.2014.04.011 (2014).
51. Molotkov. A., Mazot. P., Brewer. J. R., Cinalli. R. M. & Soriano. P. Distinct Requirements for FGFR1 and FGFR2 in Primitive Endoderm Development and Exit from Pluripotency. Dev Cell 41, 511-526 e514. doi: 10.1016/j.devcel.2017.05.004 (2017).
52. Zhu, L. et al. High-resolution Ribosome Profiling Reveals Translational Selectivity for Transcripts in Bovine Preimplantation Embryo Development. bioRx.iv, 2022.2003.2025.485883, doi: 10.1101/2022.03.25.485883 (2022).

### Example 4

Bovine Blastocyst-Like Structures Derived from Stem Cell Cultures

### Summary

Understanding the mechanisms of blastocyst formation and implantation is critical for improving farm animal reproduction but is hampered by a limited supply of embryos. Here, we developed an efficient method to generate bovine blastocyst-like structures (termed blastoids) via assembling bovine trophoblast stem cells and expanded potential stem cells. Bovine blastoids resemble blastocysts in morphology, cell composition, single-cell transcriptomes, in vitro growth, and the ability to elicit maternal recognition of pregnancy following transfer to recipient cows. Bovine blastoids represent an accessible in vitro model for studying embryogenesis and improving reproductive efficiency in livestock species.

### Introduction

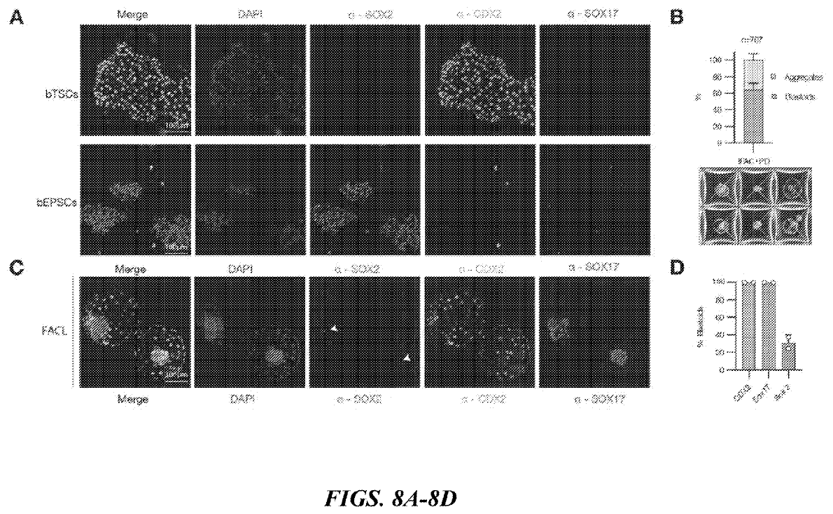

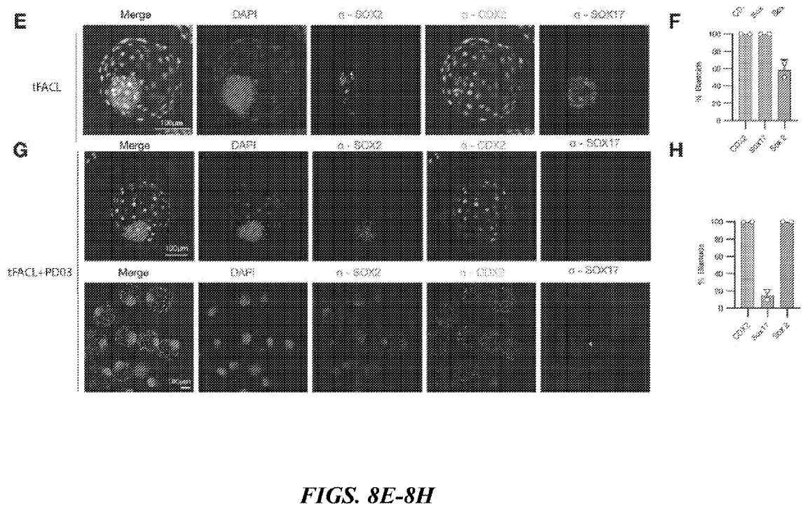

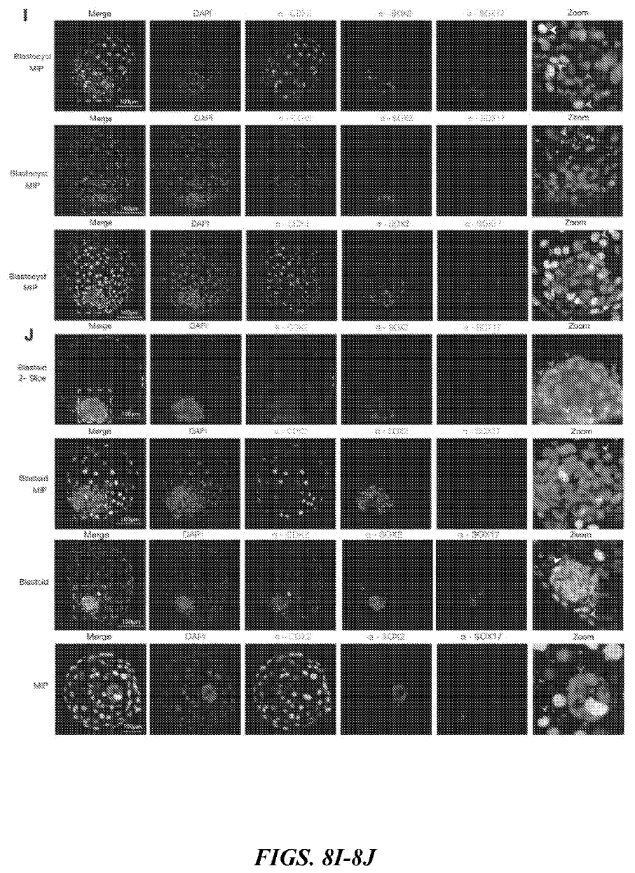

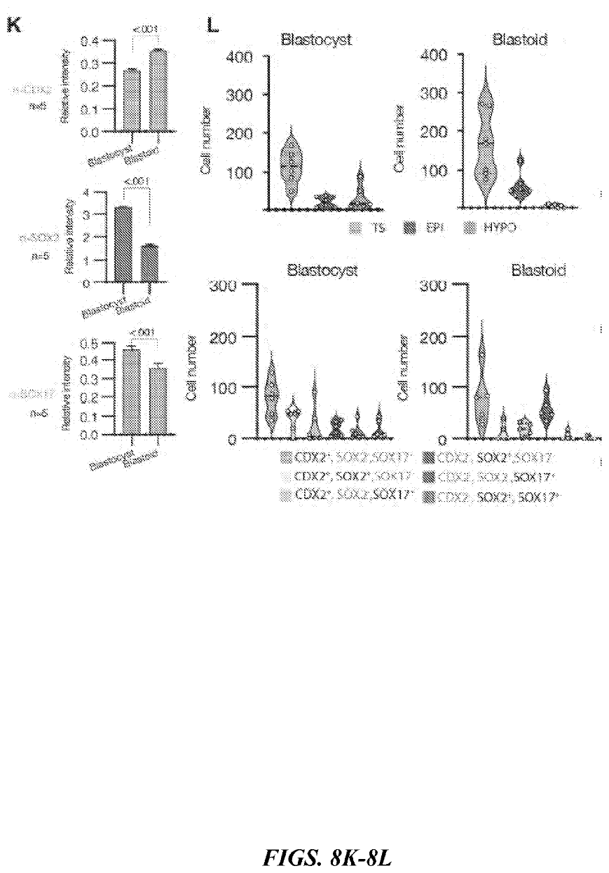

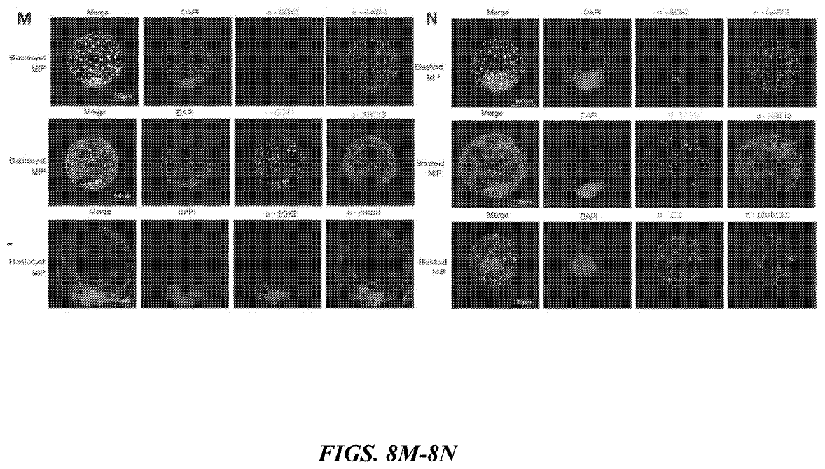

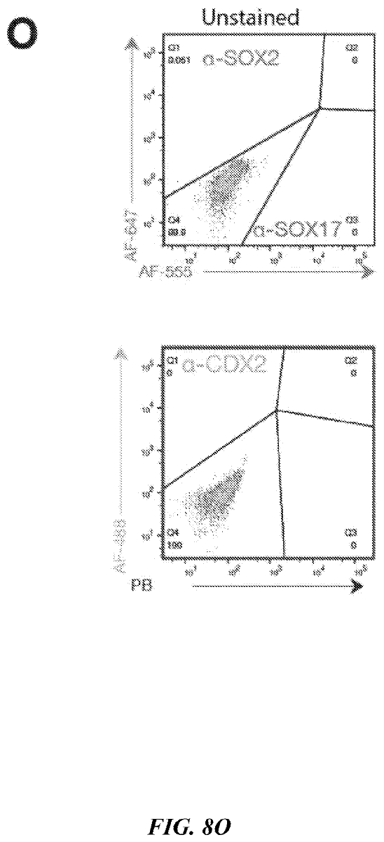

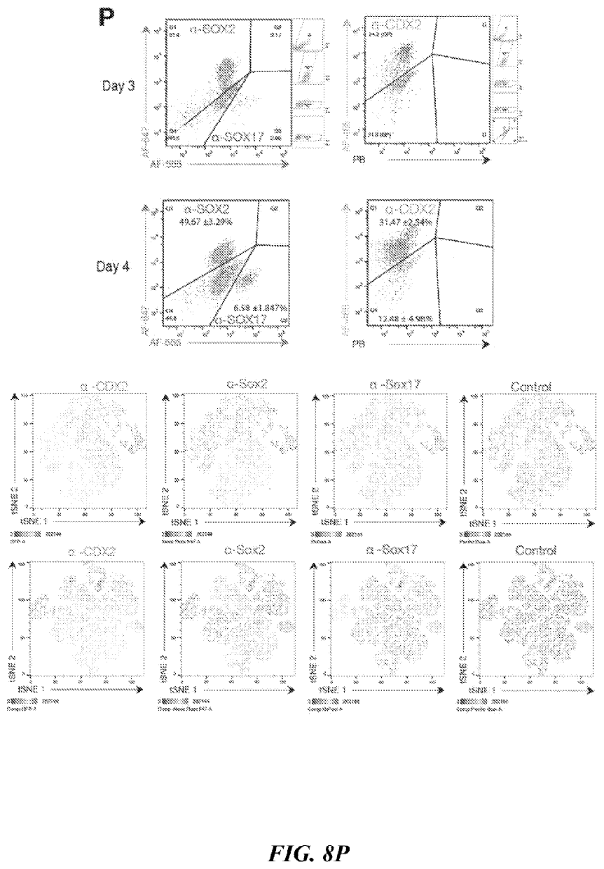

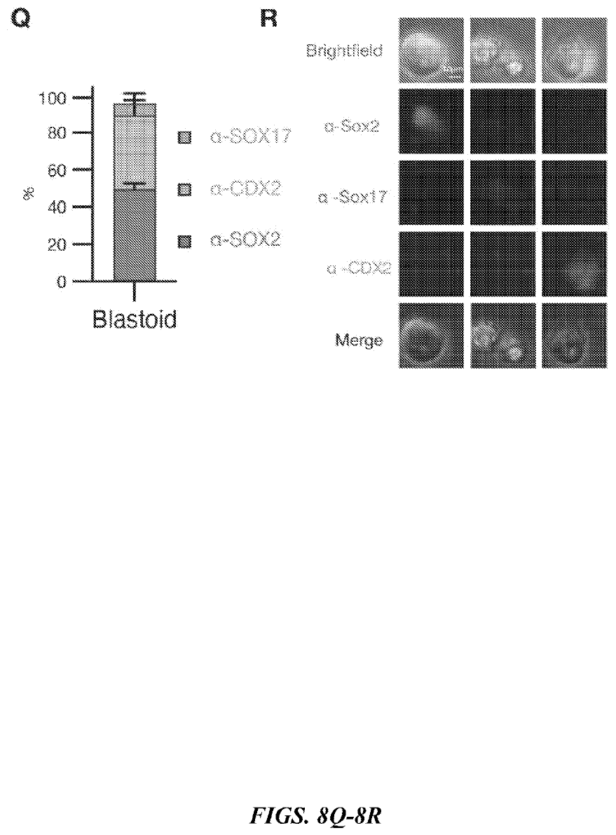

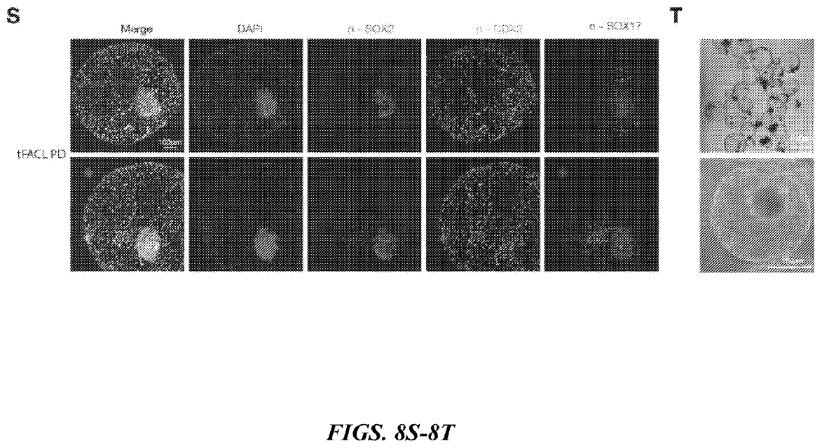

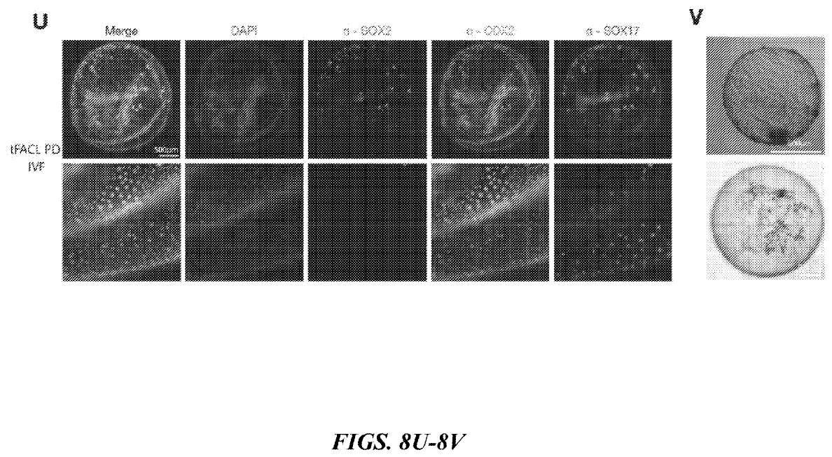

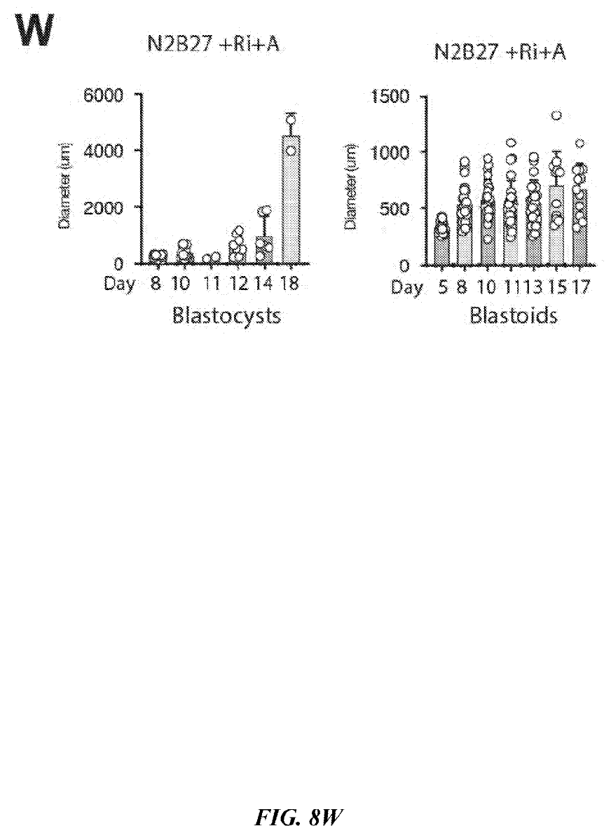

Blastoids were initially developed in mice by assembling embryonic stem cells (ESCs)¹ or extended pluripotent stem cells (EPSCs)² with trophoblast stem cells (TSCs), or through EPSC differentiation and self-organization,³ and have also been successfully generated in humans.⁴⁻⁸ To date, however, blastoids from livestock species have not been reported. Several types of pluripotent stem cells (PSCs), including EPSCs, were recently derived from Bos taurus blastocysts,⁹⁻¹⁵ which have great potential to advance animal agriculture.¹⁶ Surprisingly, we found that LCDM medium previously used for EPSC culture^{13, 17} could support de novo derivation and long-term culture of bovine TSCs¹⁸. The availability of bovine EPSCs and TSCs (FIG. 8, panel A) prompted us to test whether bovine blastoids could be generated through 3D assembly.

### Results

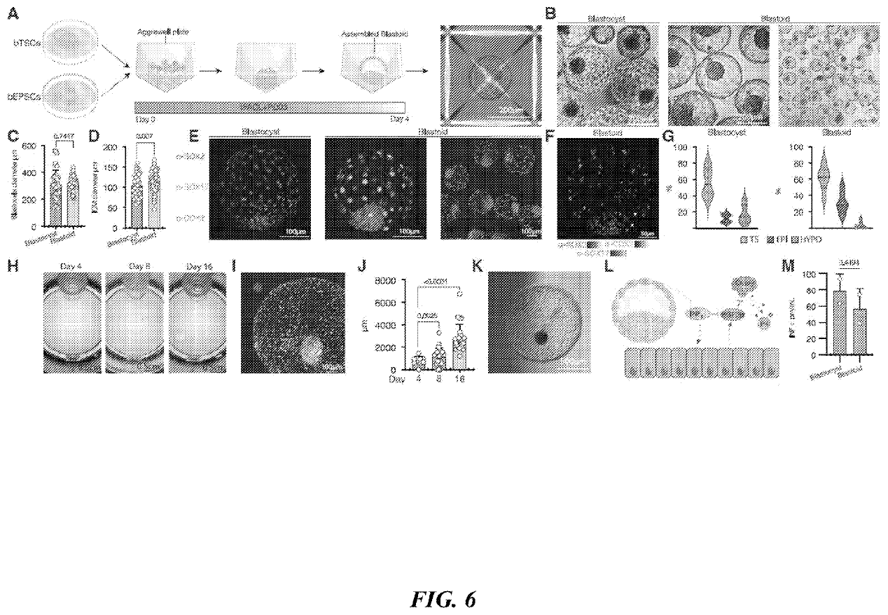

To develop a condition that supports bovine blastoid formation, we adapted the FAC (FGF2, Activin-A. and CHIR99021) medium,¹⁹ which supports the differentiation of hypoblast (HYPO)-like cells (HLCs) from naive human PSC's.^{4,20} We added the leukemia inhibitory factor (LIF) to the FAC medium that is known to improve pre-implantation bovine embryo development²¹ (FACL). FGF signaling levels can bias the fate of inner cell mass (ICM), likely acting through the RAS-RAF-MEK-ERK/MAPK pathway,^{22,23} where high levels of FGF direct ICM cells toward the HYPO (or primitive endoderm [PE]) lineage.²⁴ To support both HYPO and epiblast (EPI) lineages, we optimized FGF signaling by lowering FGF2 concentration and including a low dose of a MEK inhibitor (PD0325901, 0.3 mM), as MEK inhibition has been shown to suppress HYPO fate in bovine embryos in a dose dependent manner.²⁵ This optimized condition, termed titrated FACL+PD03 (tFACL+PD) (see STAR Methods), supported the formation of bovine blastoids with high efficiency (64.2%±7.6%) within 4 days (FIG. 6, panel A, panel B; FIG. 8, panels B-H, panel O, and panel P).

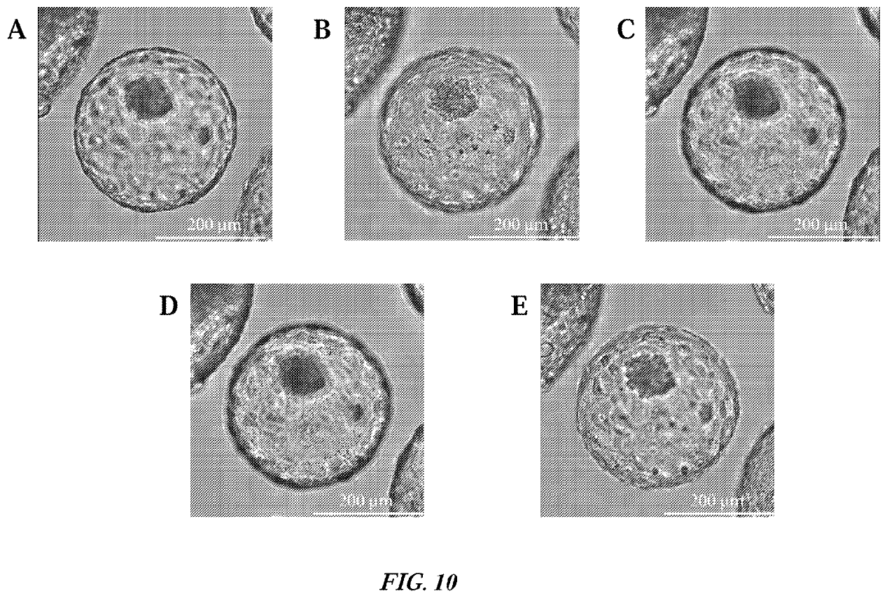

Morphologically each bovine blastoid contains a blastocelelike cavity, an outer trophectoderm (TE)-like layer, and an ICMlike compartment, which resembles bovine blastocysts produced by in vitro fertilization (IVF) (FIG. 6, panel B; FIG. 10). Cavity and ICM sizes of day-4 bovine blastoids reached diameters equivalent to day-8 IVF blastocysts (FIG. 6, panel C and panel D). We performed immunofluorescence (IF) analysis and found that bovine blastoids contained cells that expressed markers characteristic of EPI (SOX2), HYPO (SOX17), and TE (GATA3, KRT18, and CDX2) lineages (FIG. 6, panel E; FIG. 8, panel J), and stained positive for a tight junction marker ZO1 (TJP1) and an apical marker F-actin (Phalloidin), comparable to blastocysts (FIG. 8, panel M and panel N). Despite the similarities, we found that the expression levels of lineage markers were different between IVF blastocysts and blastoids when quantified via IF, with blastoid-trophoblast-like cells (TLCs) expressing higher levels of CDX2. HLCs expressing lower levels of SOX17 and EPI-like cells (ELCs) expressing lower levels of SOX2 when compared to their corresponding cell types in IVF blastocysts (FIG. 8, panels I-K). While the proportion and cell number for TE were comparable between blastoids and blastocysts, as revealed by confocal microscopy. 3D reconstruction, and spots colocalization (FIG. 6, panel F), we found IVF blastocysts contained a higher number of HYPO cells and cells co-expressing different lineage markers and a lower number of EPI cells than blastoids (blastoids: 30.87%±13.11% [ELCs]. 61.71%±15.54% [TLCs], 3.61%±4.41% [HLCs]; blastocysts: 9.7%±5.02% [EPI], 54.18%±18.34% [TE], 15.27%±11.59% [HYPO]) (FIG. 6, panel F; FIG. 8, panel L).

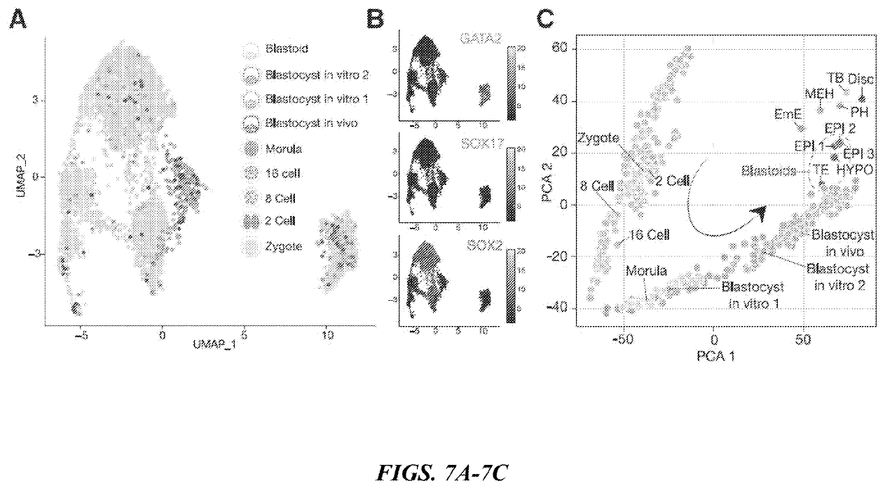

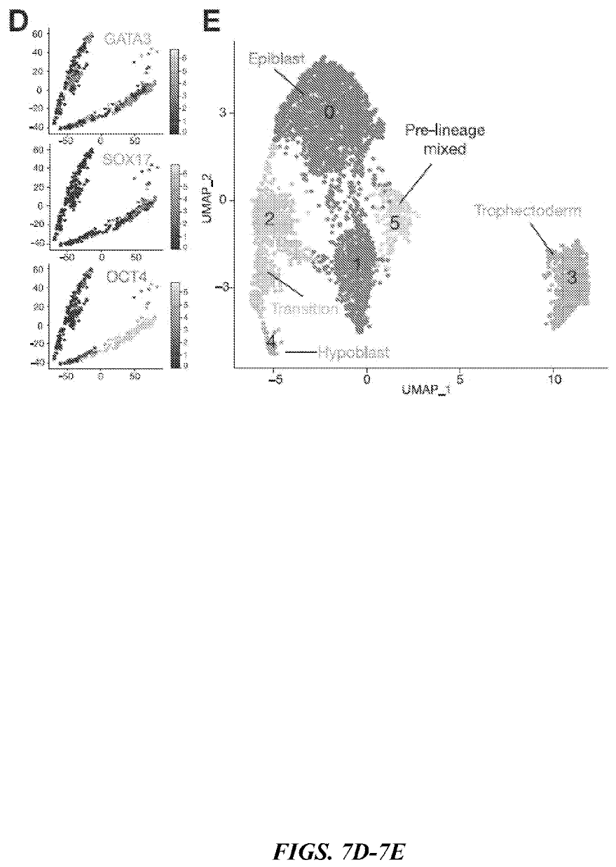

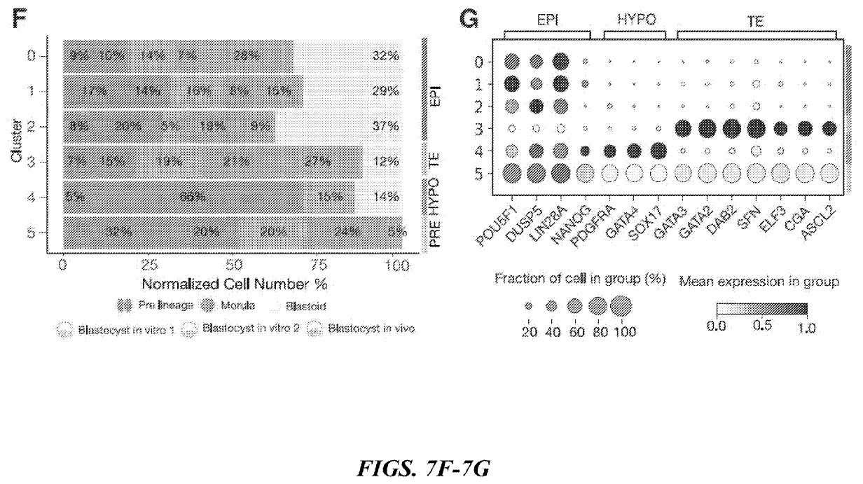

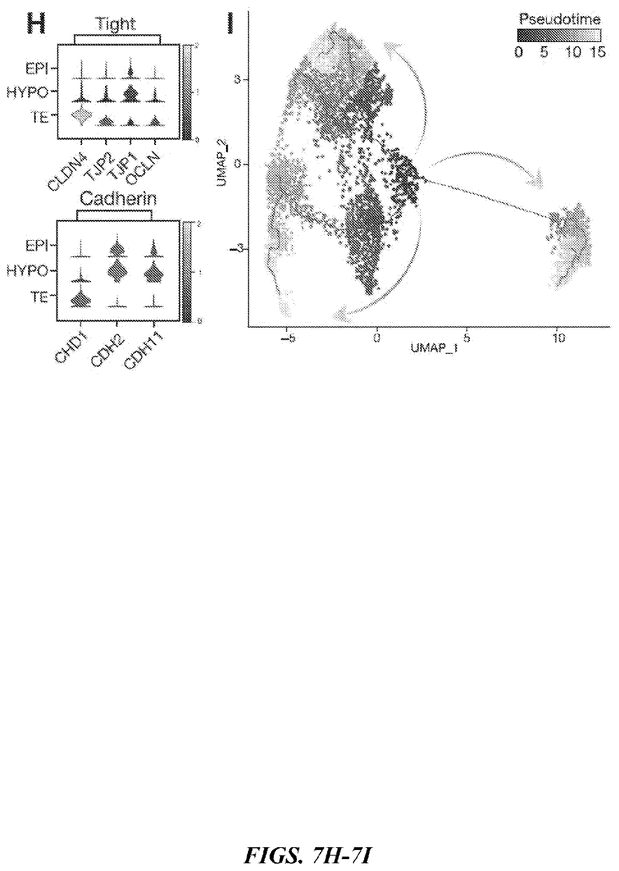

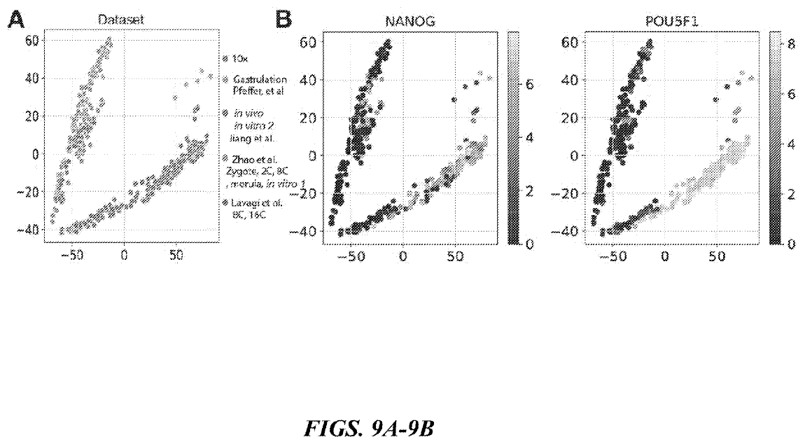

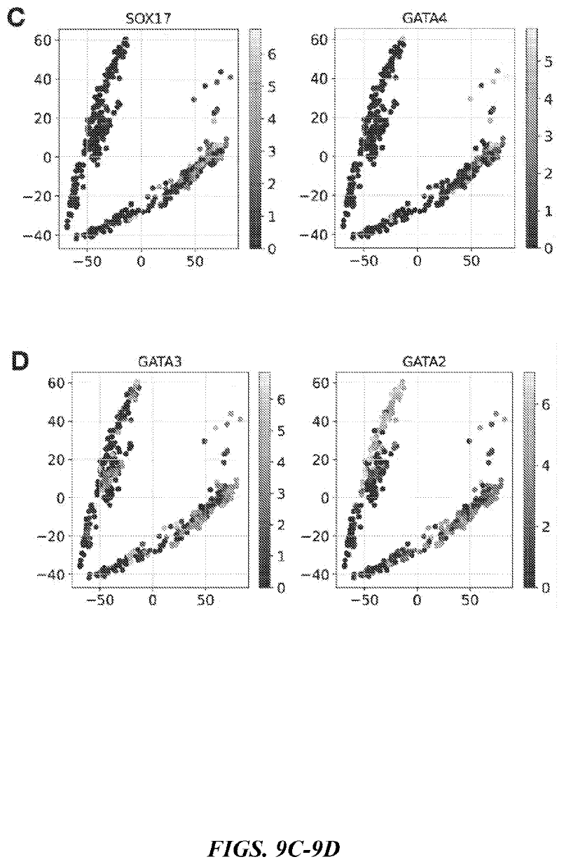

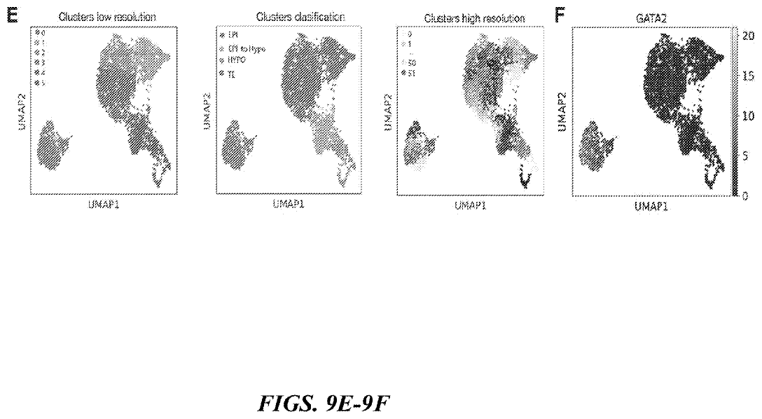

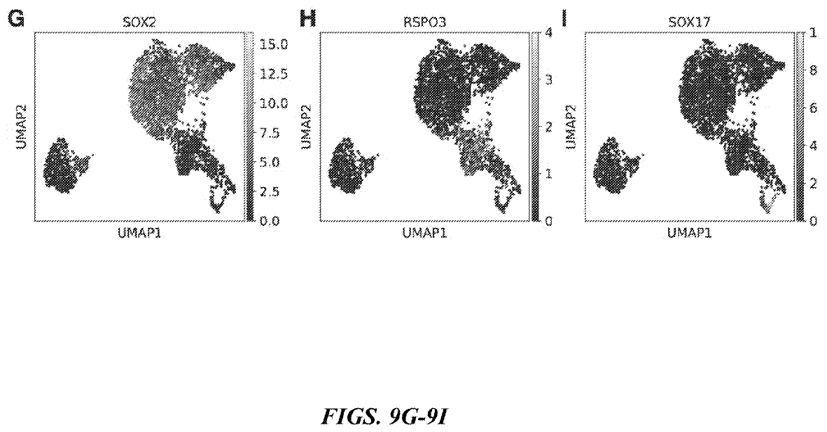

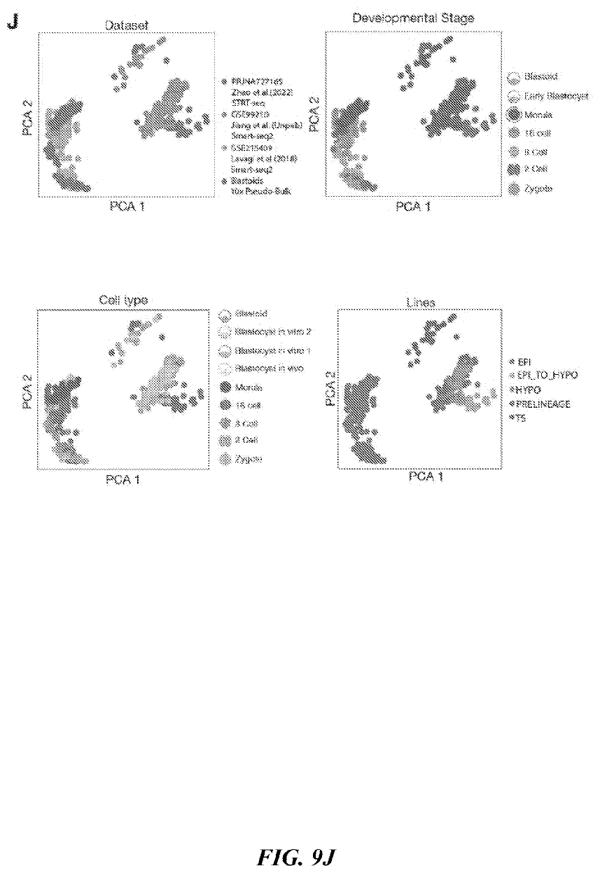

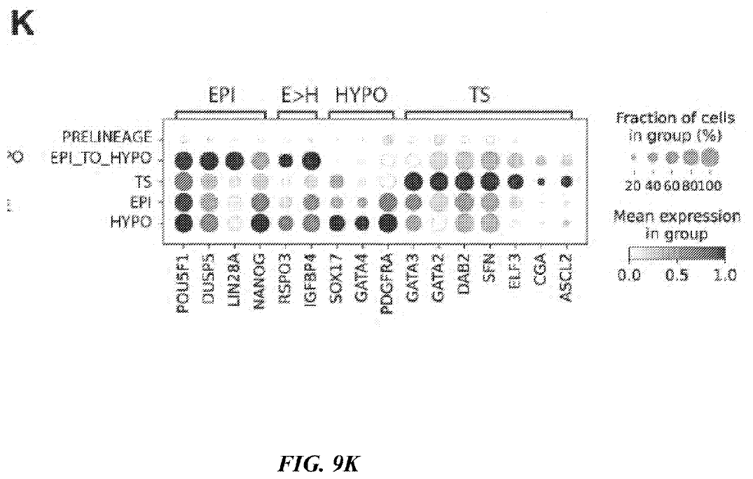

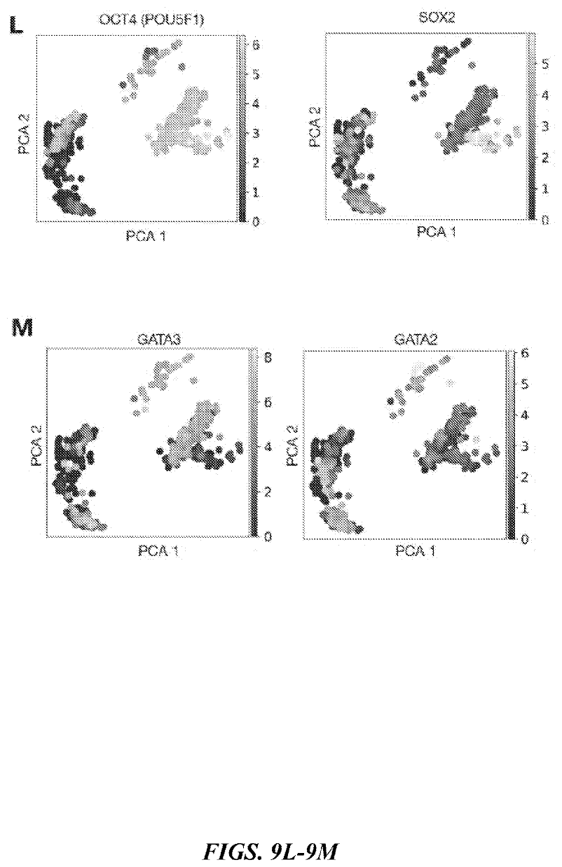

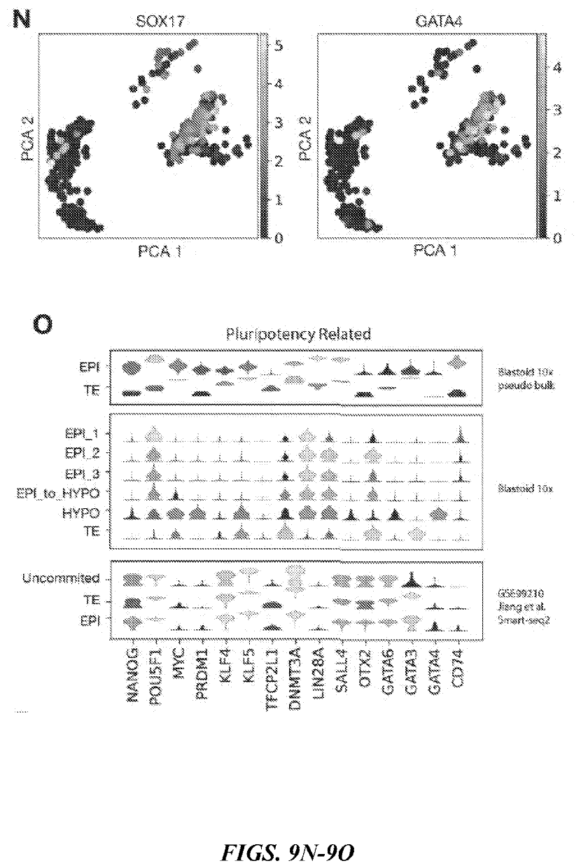

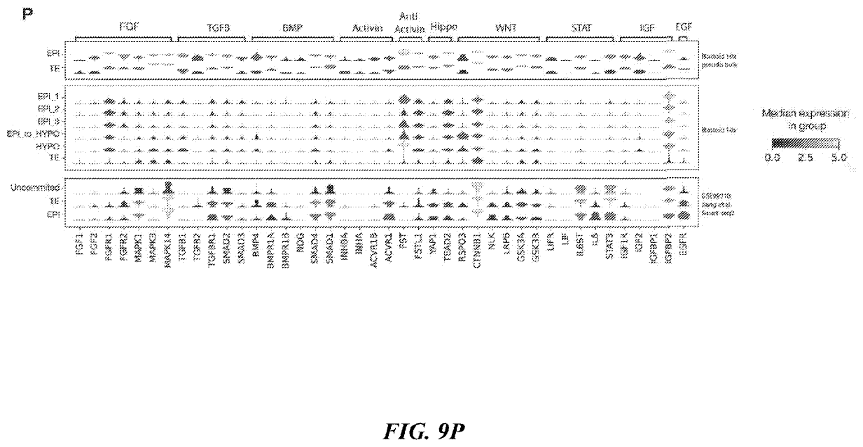

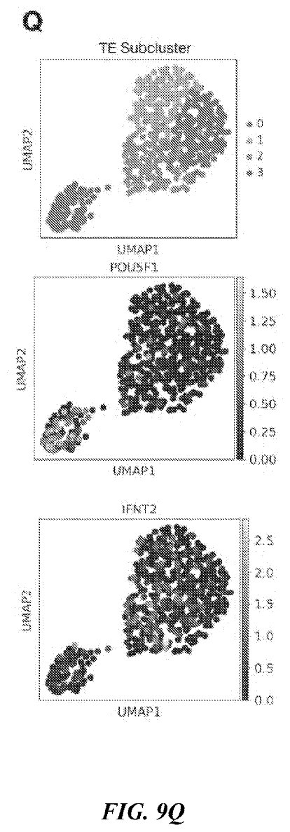

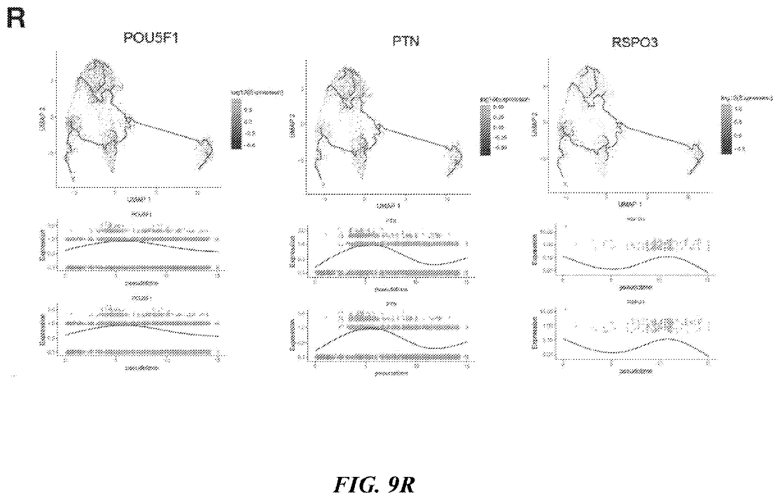

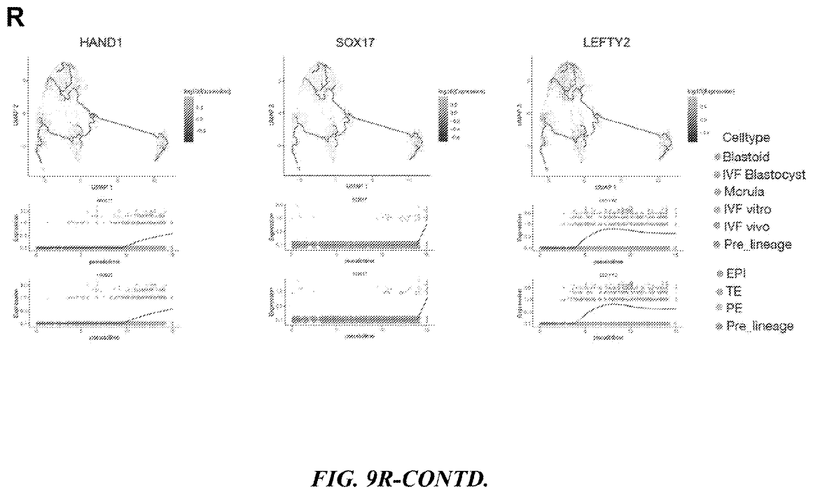

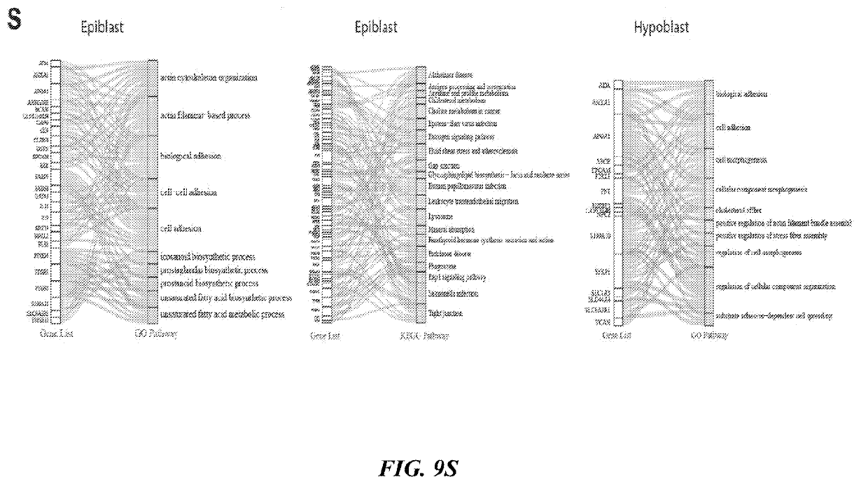

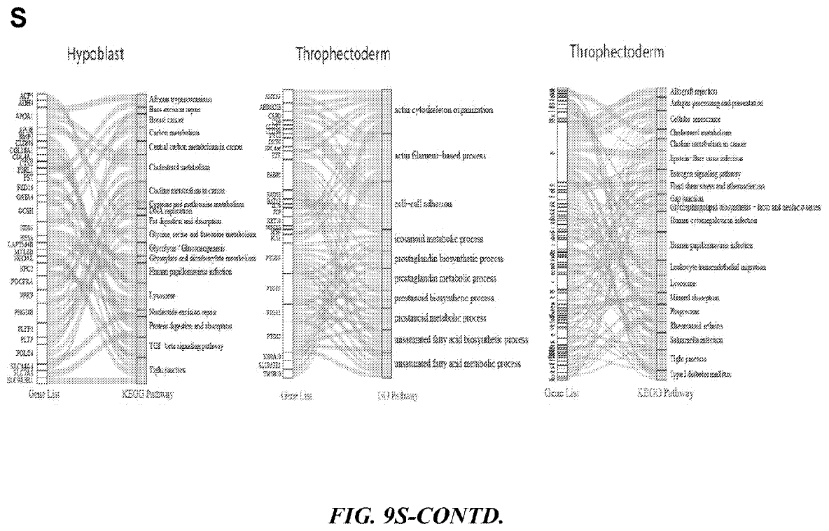

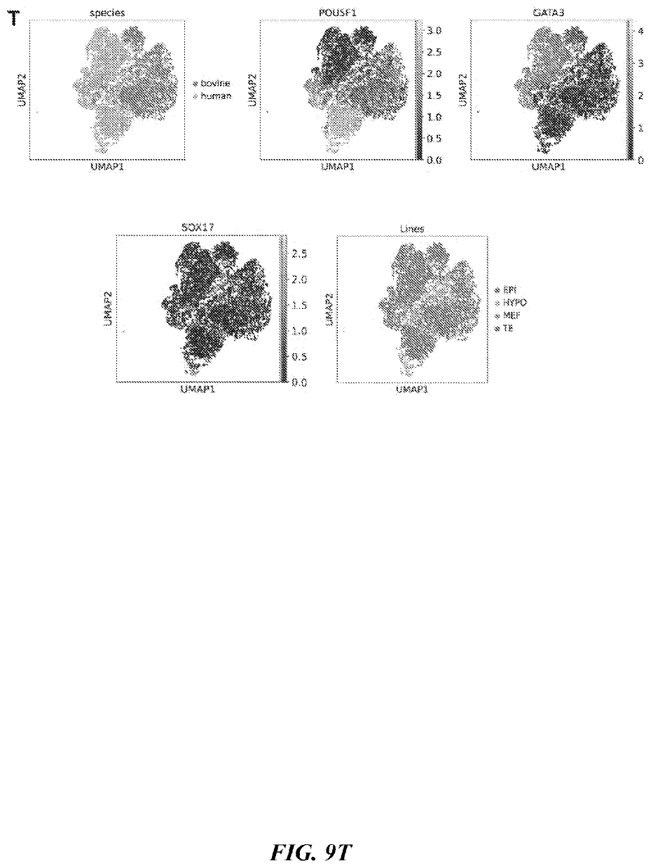

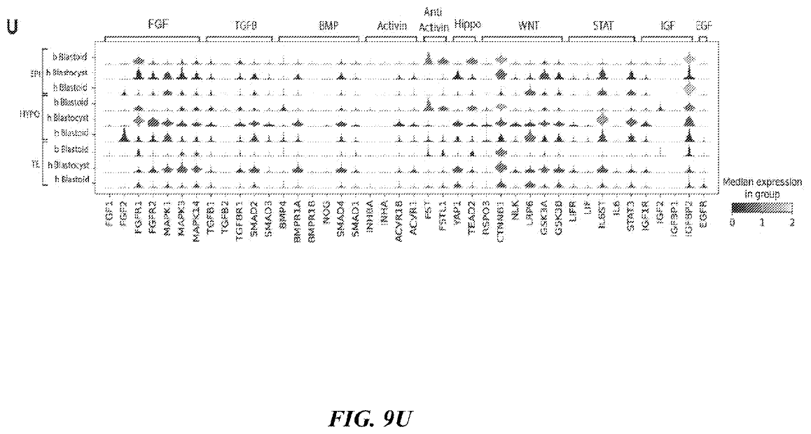

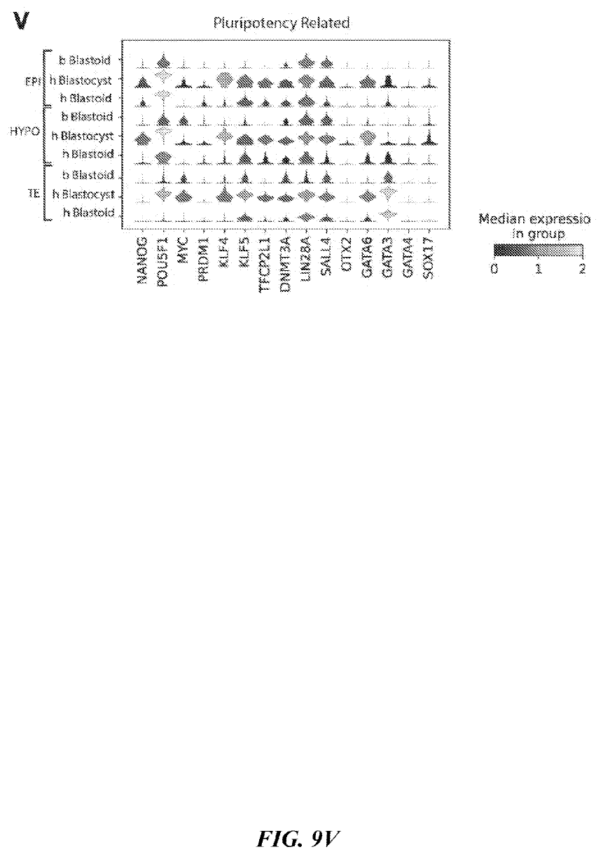

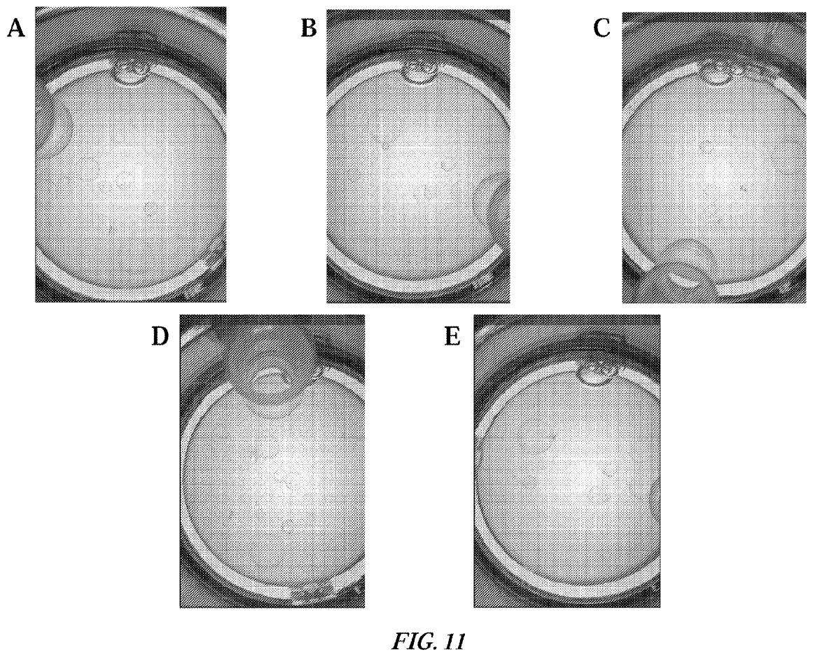

Next, we evaluated the in vitro growth of blastoids and blastocysts under a 3D suspension culture (ClinoStar, see STAR Methods). We found trophoblast cells and cavities in both IVF blastocysts and blastoids continued to proliferate and expand for more than 2 weeks, which were also accompanied by an increase in the ICM size (FIG. 6, panels H-K; FIG. 8, panels S-W; FIG. 11). We performed embryo transfer to synchronized surrogates to evaluate whether blastoids can establish maternal interaction and pregnancy (see STAR Methods). Interestingly, we detected the anti-luteolytic hormone interferon-tau (INFt) in surrogates' blood.²⁶ INFt is the signal for maternal recognition of pregnancy in ruminants, which acts by blocking prostaglandin (PGF) release from the uterus and allowing the corpus luteum (CL) to persist and the pregnancy to be maintained²⁷⁻²⁹ (FIG. 6, panel L). INFt was measured at concentrations of 56.53±25.13 μm/mL in 2 out of 4 surrogates 7 days following blastoid transfer, which were comparable to those from IVF blastocyst transfers (78.36±21.54 μm/mL) in 2 out of 5 surrogates (FIG. 6, panel M). To determine the transcriptional states of bovine blastoid cells, we performed single-cell RNA sequencing (scRNA-seq) using the 103 Genomics Chromium platform and carried out integrated analysis with Smart-seq2 single-cell transcriptomes derived from zygote,³⁰ 2 cell,³⁰ 8 cell,³¹ 16 cell,³¹ morula,³⁰ and two sets of day 7.5 blastocyst stage IVF bovine embryos³⁰ as well as in vivo produced bovine blastocysts (see data and code availability). Joint uniform manifold approximation and projection (UMAP) embedding revealed blastoid-derived cells clustered with blastocyst-derived cells (FIG. 7, panel A and panel B). To further evaluate the temporal identity of blastoid cells, we performed two pseudo bulk analysis on the 103 blastoid data at a low and a high cluster resolution, to compensate for the differences in sequencing depth to Smart-seq2 data (FIG. 7, panel C and panel D; FIG. 9, panels A-P). For the first analysis, we also included datasets from bovine early gastrulation-stage embryos.³² We found that different embryo datasets were orderly arranged on the principal component analysis (PCA) plot according to their developmental time, and blastoid cells were mapped closer to blastocyst cells (FIG. 7, panel C and panel D; FIG. 9, panels A-D).

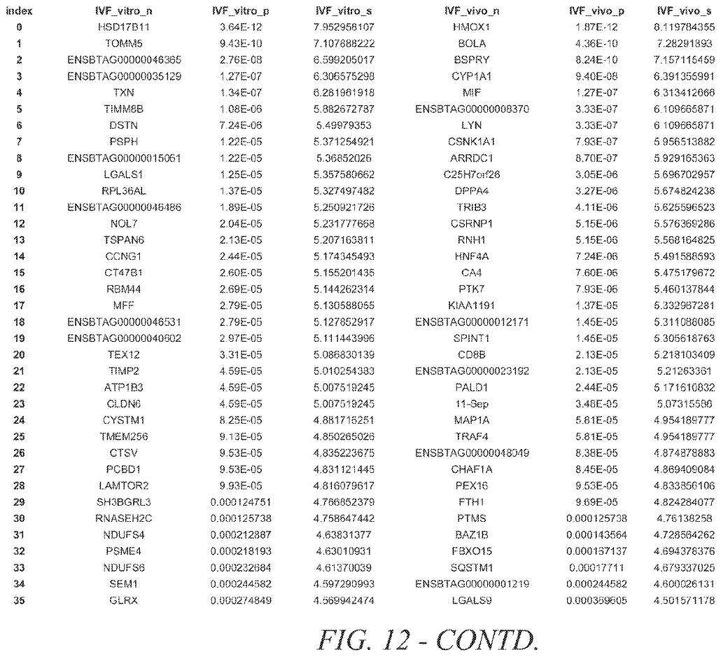

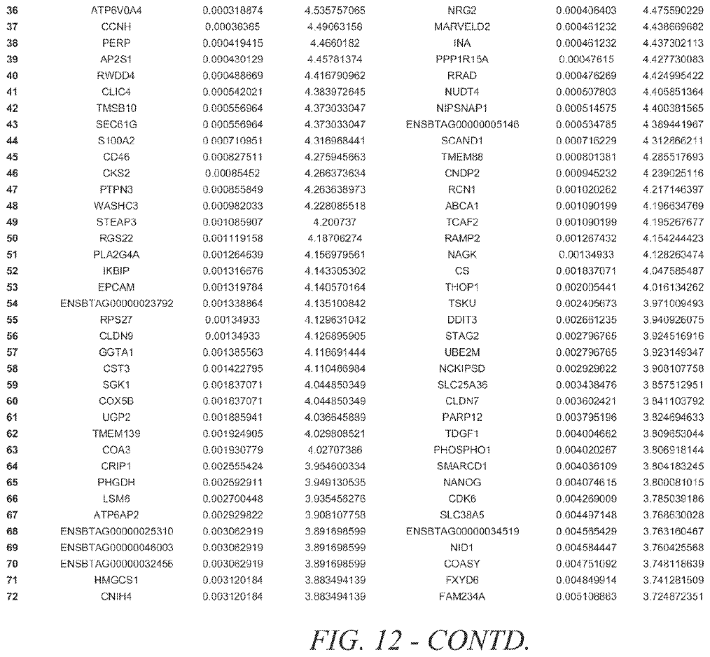

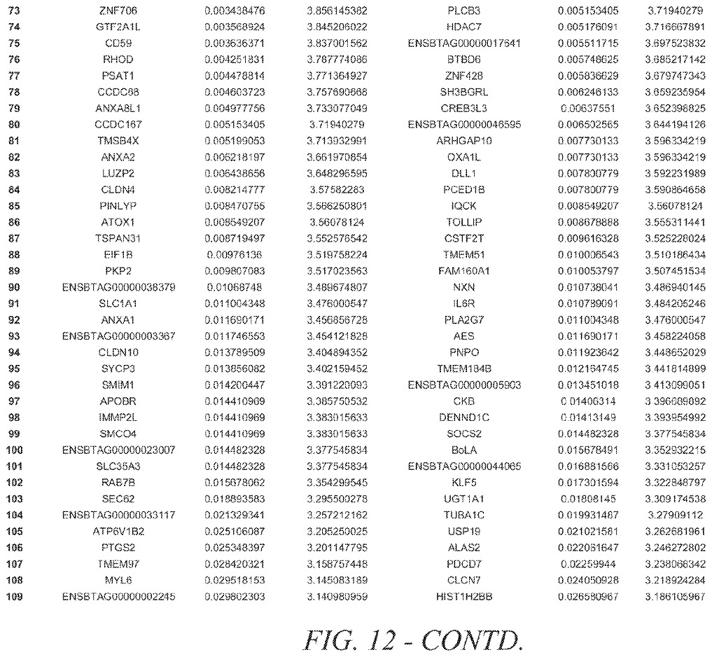

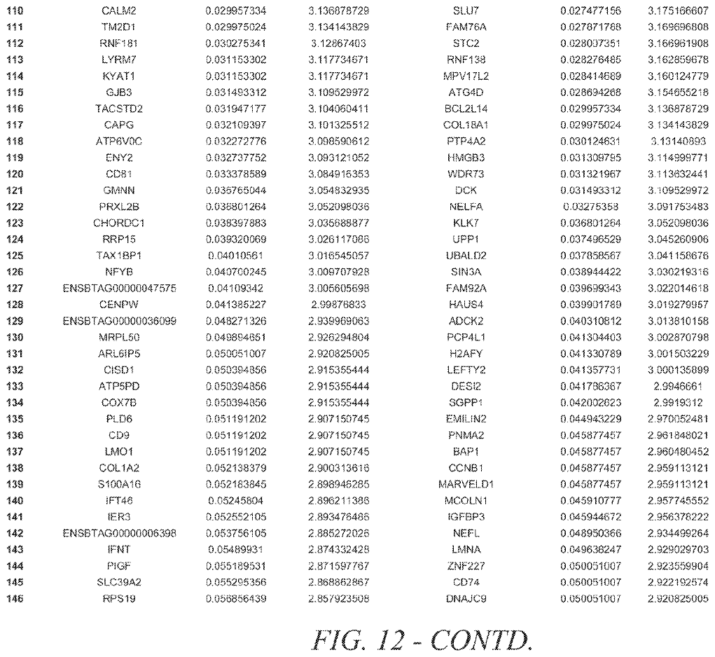

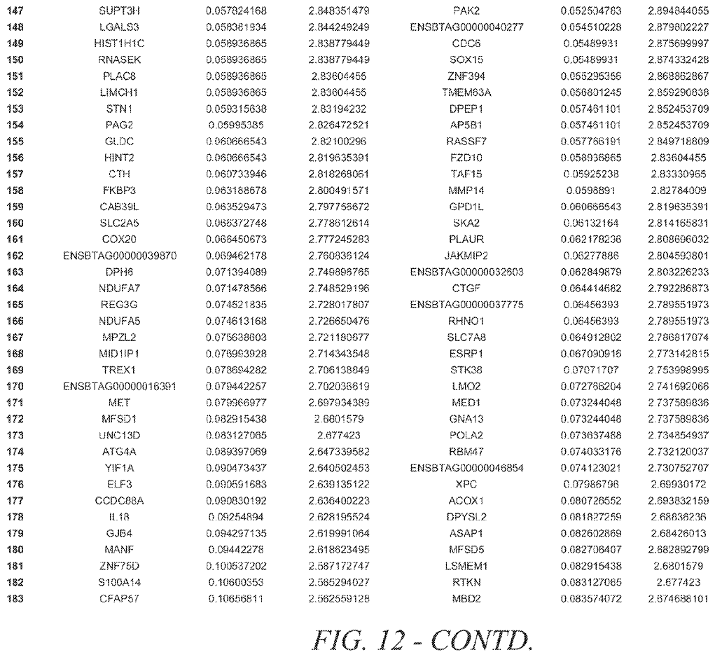

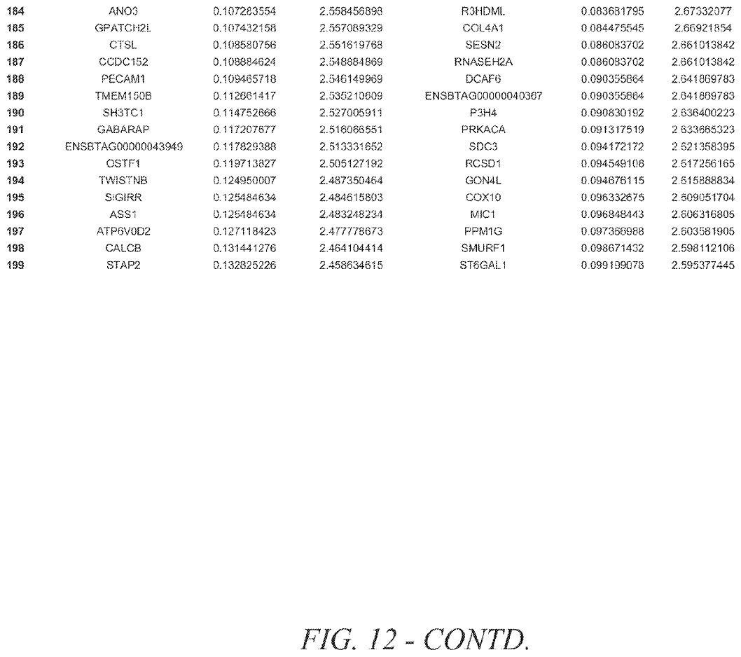

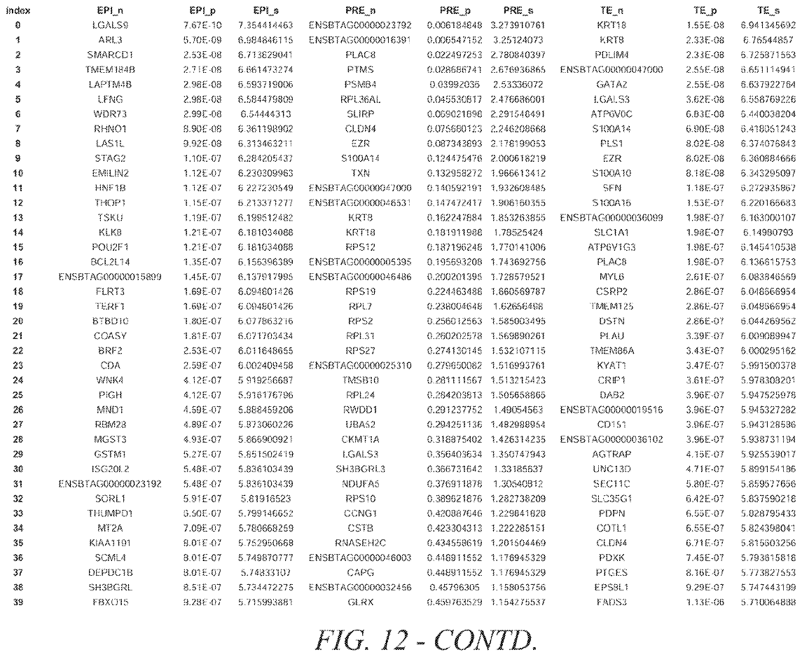

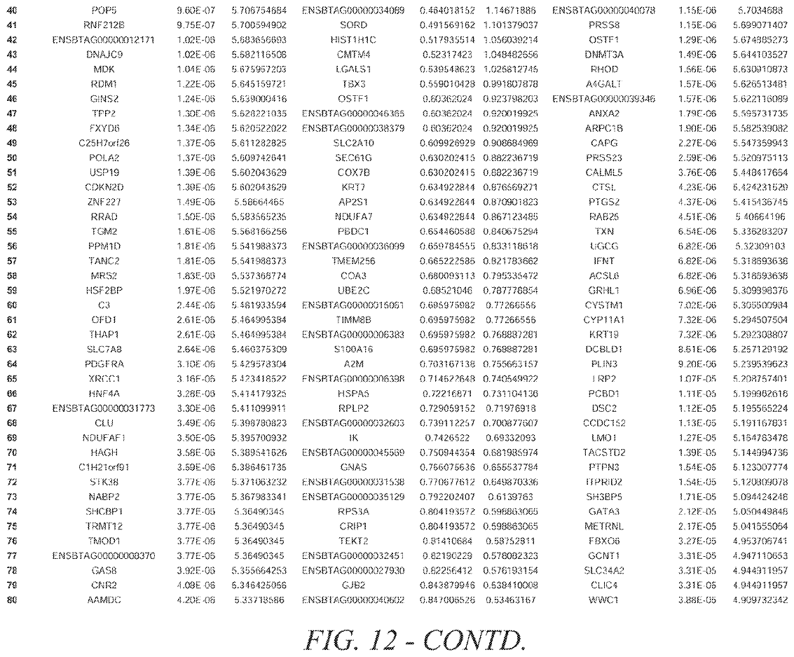

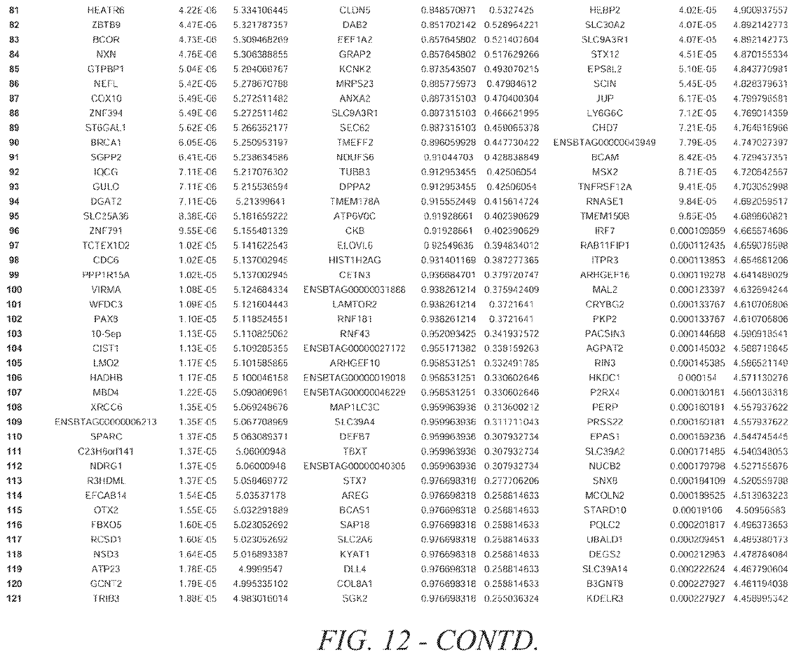

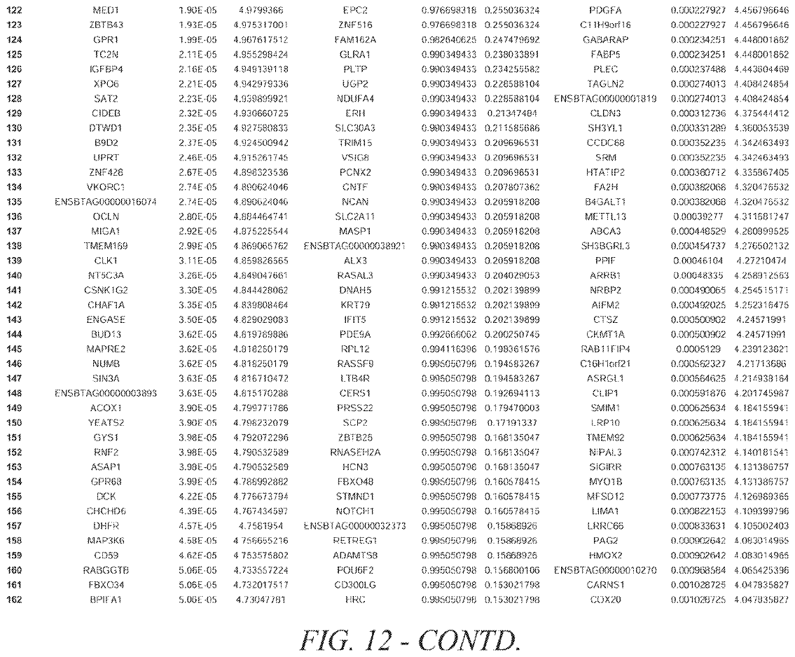

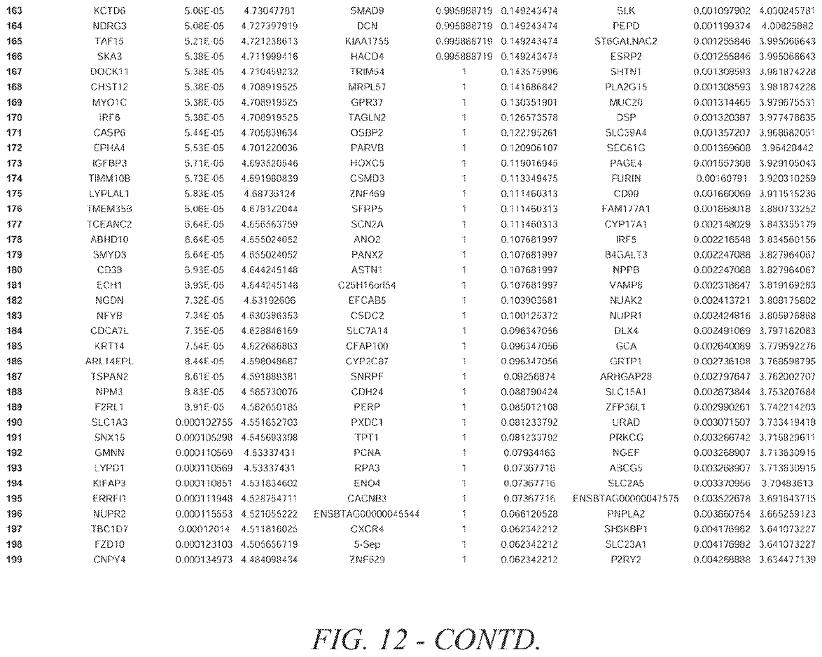

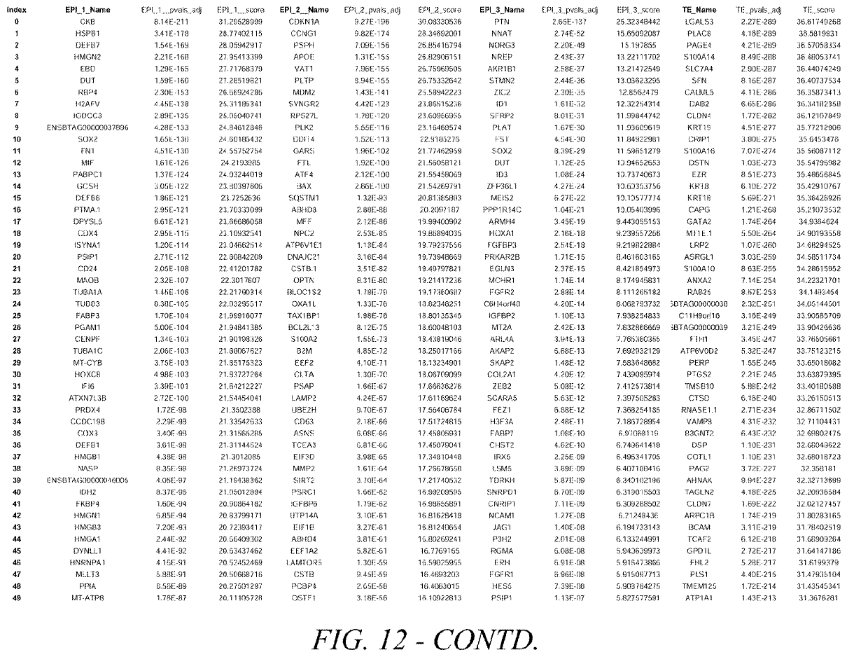

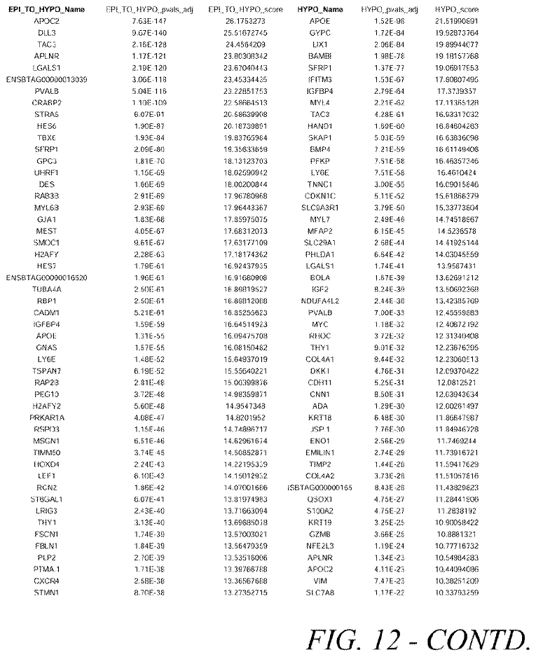

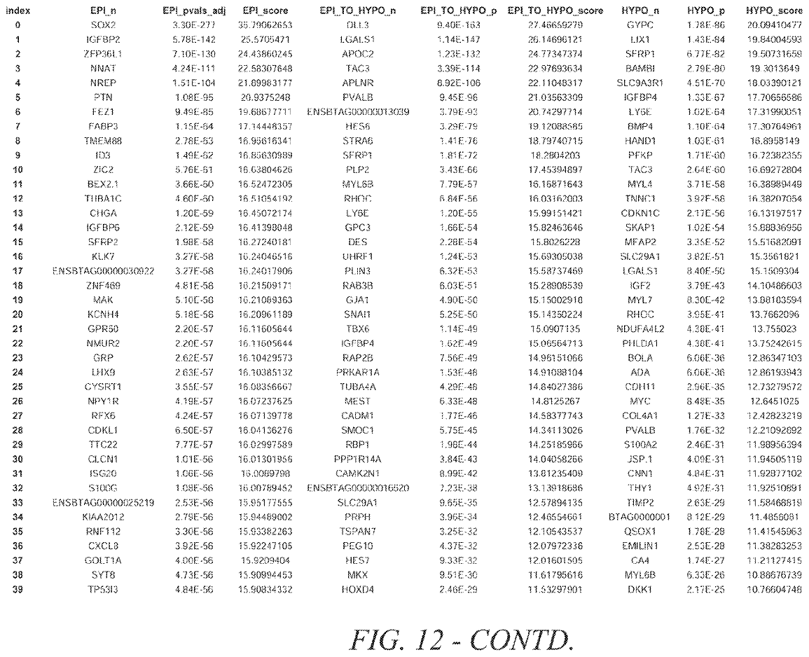

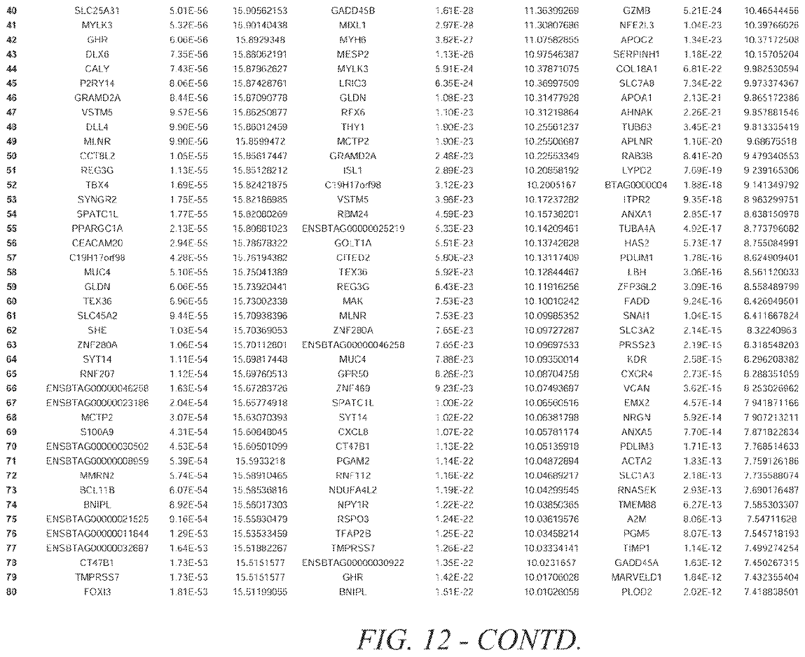

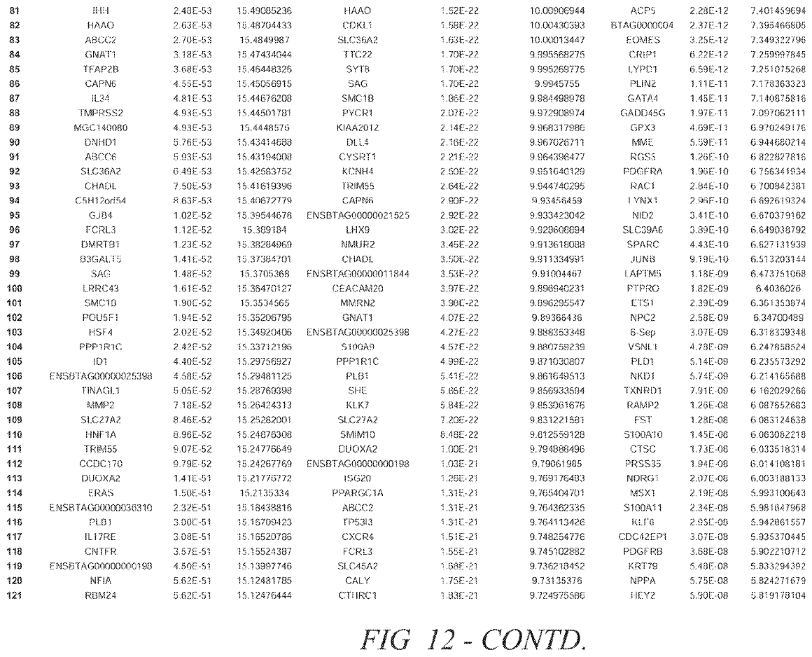

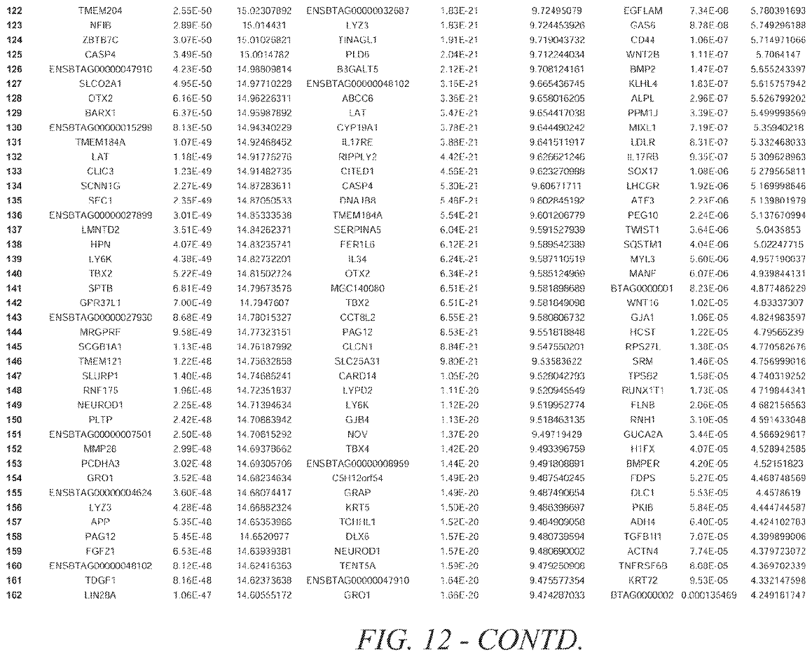

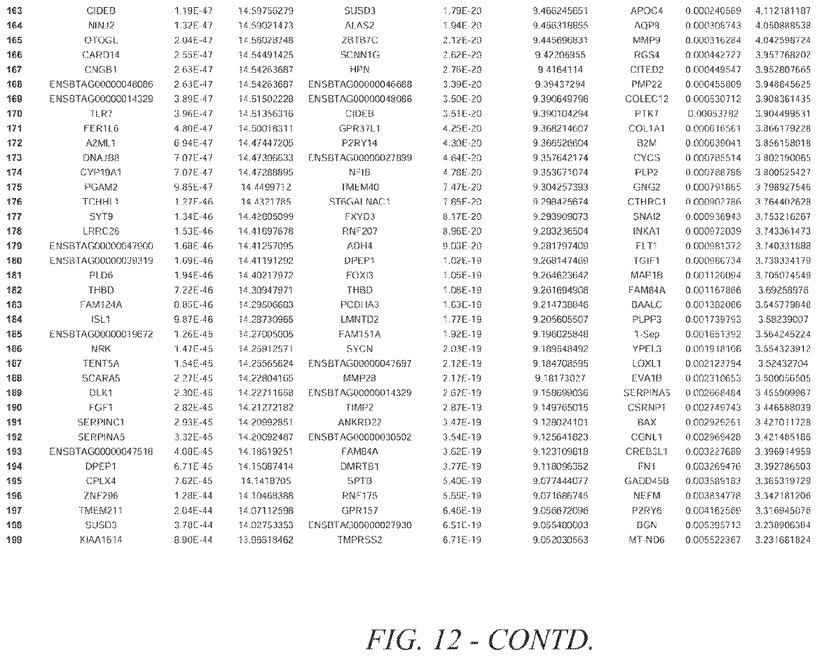

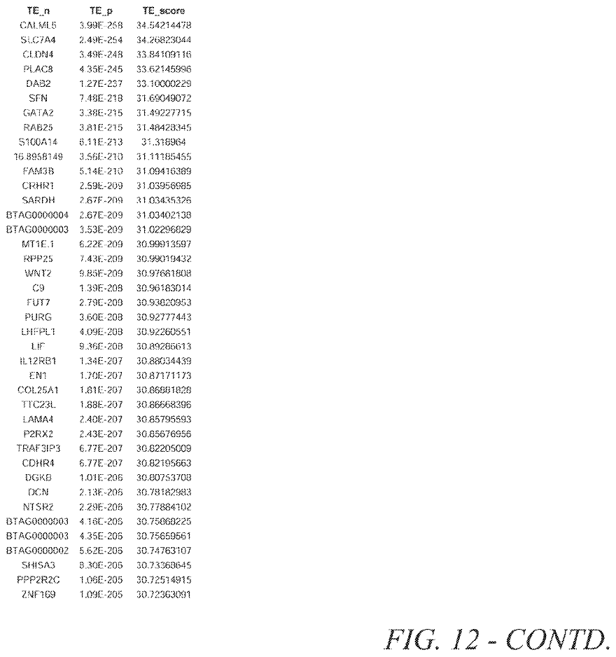

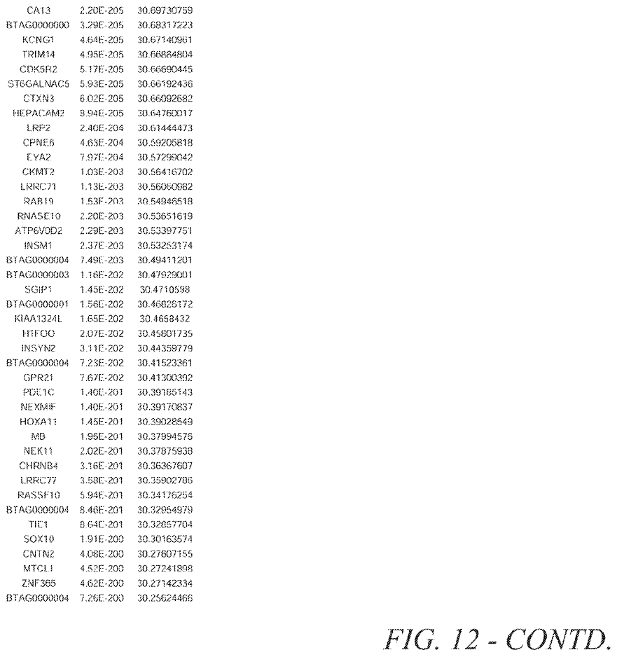

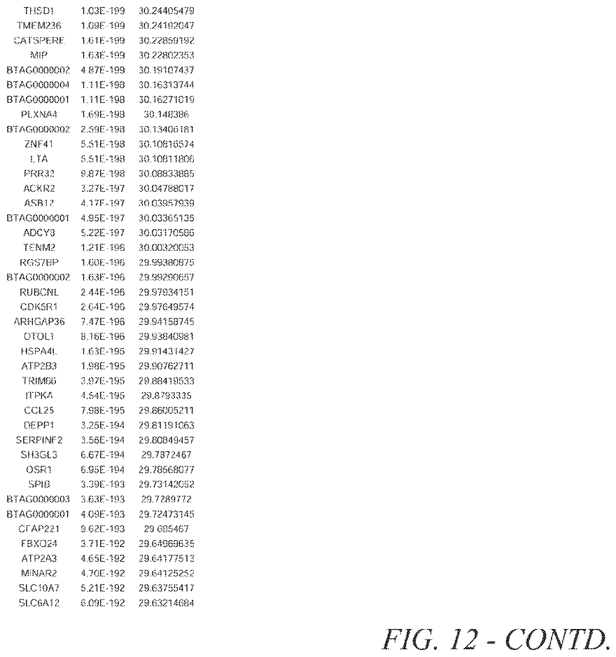

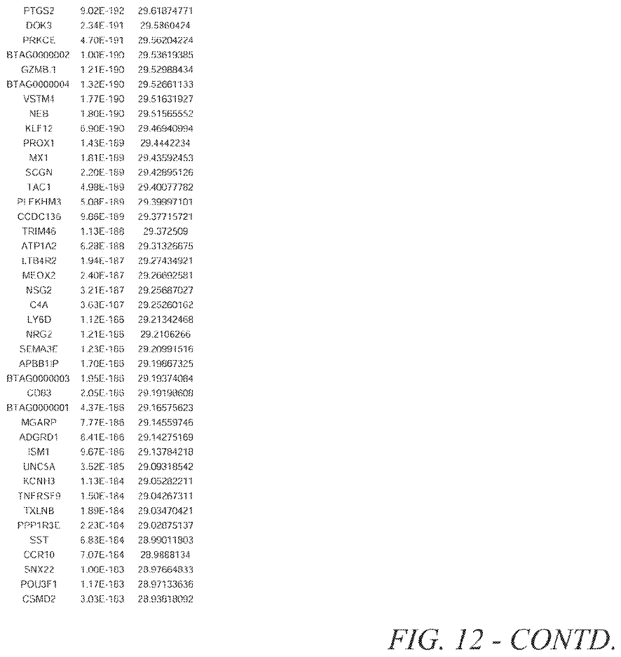

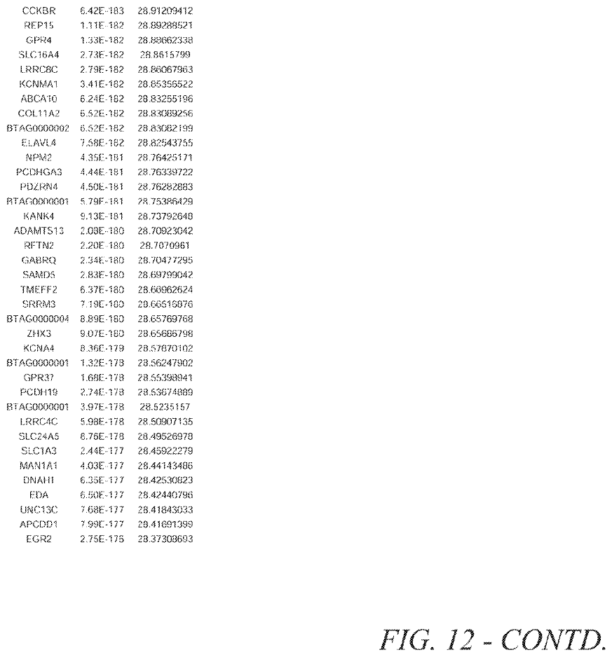

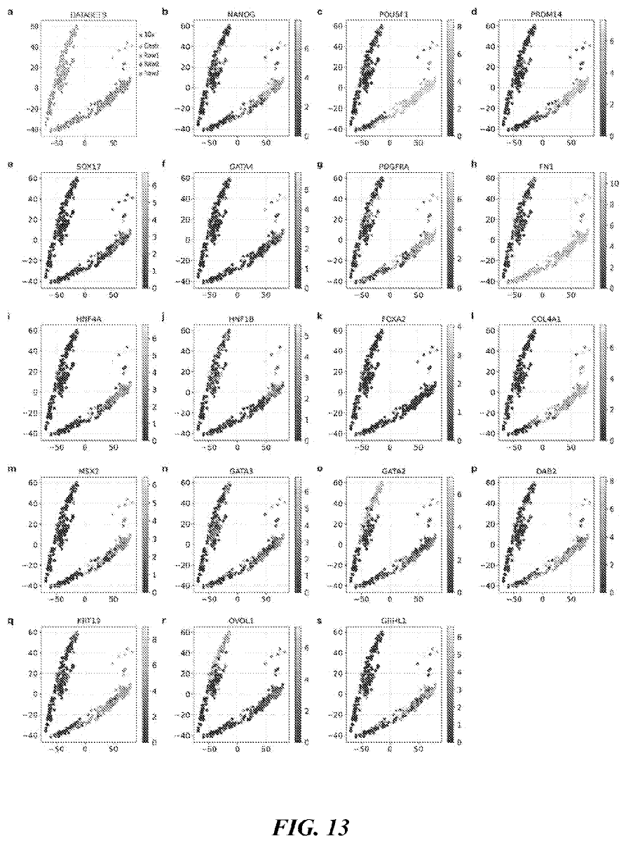

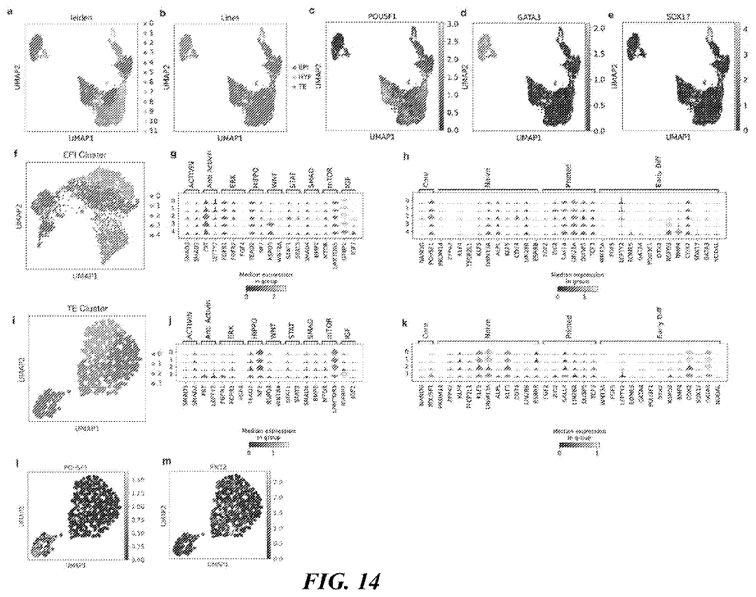

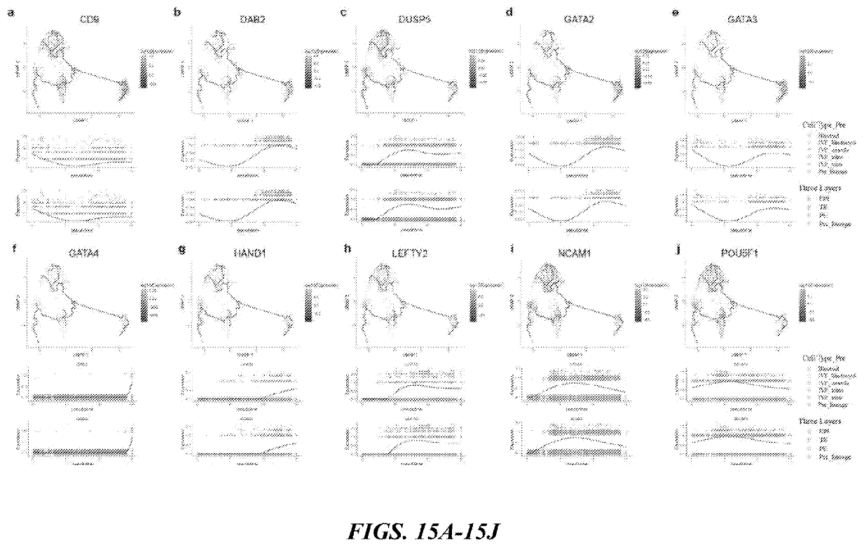

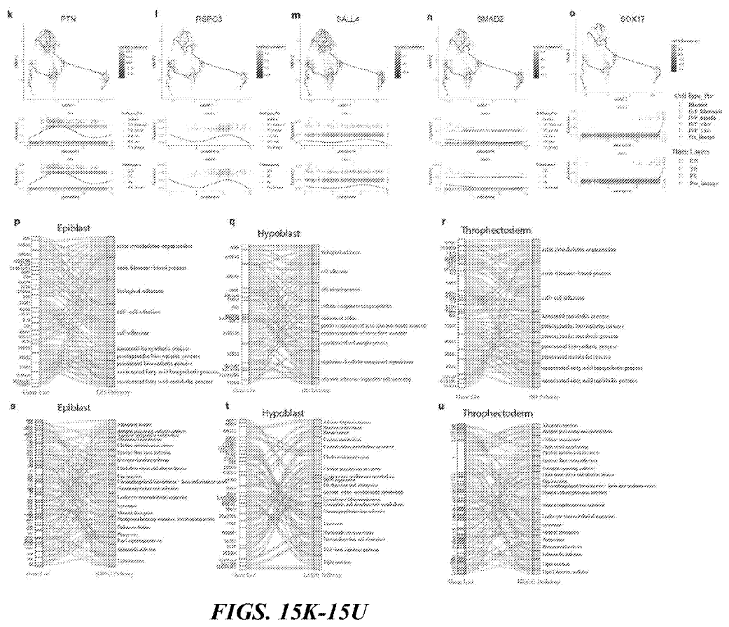

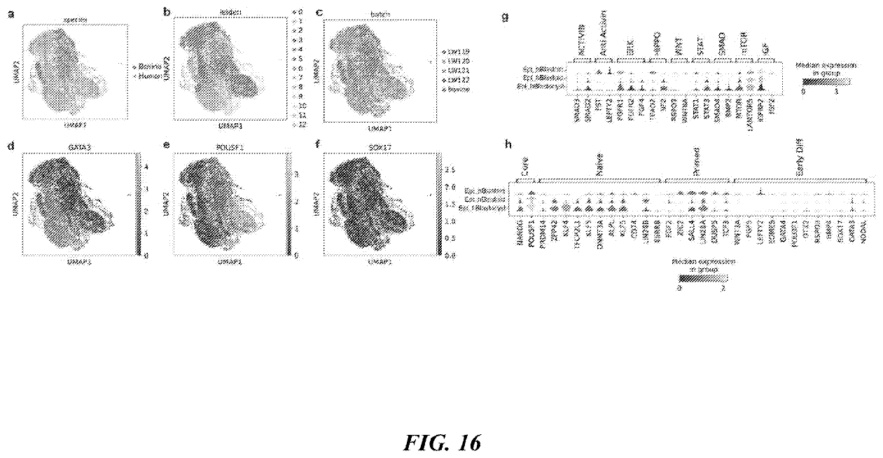

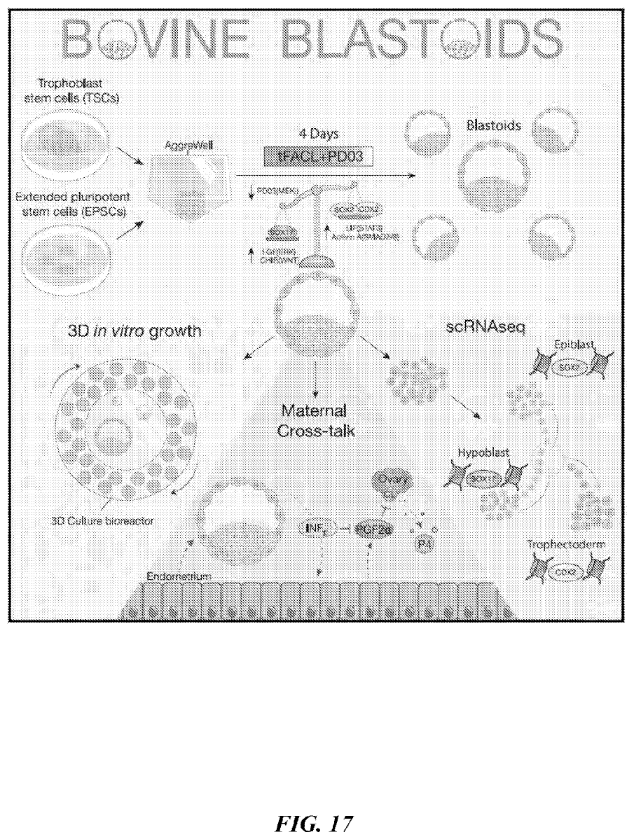

We annotated the six identified cell clusters based on marker gene expression and overlap with cells from bovine embryos (FIG. 7, panels E-G; FIG. 12). Cluster 3 expresses TE markers, e.g., GATA2 and GATA3, and is annotated as TLCs; cluster 4 expresses HYPO markers, e.g., GATA4 and SOX17, and thus represents HLCs; three clusters (0, 1, and 2) express EPI markers, e.g., SOX2 and LIN28a, and are designated as ELCs; cluster 5 is mostly composed of cells from pre-blastocyst stage embryos (named pre-lineage), and each blastoid cluster expressed lineage-specific cadherin and tight junction markers (FIG. 7, panels E-H). To evaluate the relationship between clusters, we performed pseudotime analysis, which predicted the differentiation trajectories from pre-lineage cluster to blastocyst and blastoid lineages, showing markers of EPI to HYPO transitioning cells such as RSPO333,34 (FIG. 7, panel E and panel 1; FIG. 9, panel H and panel R). Finally, cross-species comparison revealed similarities and differences of bovine blastoids with human blastoids and blastocysts (FIG. 9, panels T-V).

### Discussion

Here we report an efficient and robust protocol to generate bovine blastoids by assembling EPSCs and TSCs that can self-organize and faithfully recreate all blastocyst lineages. Bovine blastoids show a resemblance to bovine blastocysts in morphology, size, cell number, lineage composition, and could produce maternal recognition signal upon transfer to recipient cows. Bovine blastoids represent a valuable model to study early embryo development and understand the causes of early embryonic loss. Upon further optimization, bovine blastoid technology could lead to the development of new artificial reproductive technologies for cattle breeding, which will allow for a paradigm shift in livestock reproduction

KEY RESOURCES TABLE

| | |
|---|---|
| REAGENT or | |
| RESOURCE | SOURCE |

ANTIBODIES

| | |
|---|---|
| Anti-CDX-2, Clone | BioGenex |
| CDX2-88 antibody | |
| Human SOX17 Affinity | R&D Systems |
| Purified | |
| Polyclonal Ab antibody | |
| Sox-2 (E-4) antibody | Santa Cruz Biotechnology |
| Anti-Human SOX2 | BioGenex |
| GATA3-human | Santa Cruz Biotechnology |
| antibody | |
| Keratin 18 | Sigma |
| Anti-ZO-1 Monoclonal | Innovate Research |
| Antibody | |
| Alexa Fluor ® 594 | Invitrogen |
| Phalloidin antibody | |
| Phospho-Stat3 (Tyr705) | Cell Signaling Technology |
| (D3A7) | |
| Donkey anti-rabbit IgG | Invitrogen |
| (H + L) | |
| Antibody, Alexa Fluor ™ | |
| 647 | |
| Donkey anti-mouse IgG | Invitrogen |
| (H + L) | |
| Antibody, Alexa | |
| Fluor ™488 | |
| Donkey anti-Goat IgG | Invitrogen |
| (H + L) | |
| Antibody, Alexa Fluor ™ | |
| 555 | |

CHEMICALS, PEPTIDES, AND RECOMBINANT PROTEINS

| | |
|---|---|
| N2 supplement (100X) | Gibco |
| B27 supplement (50X) | Gibco |
| Insulin-Transferrin- | Gibco |
| Selenium- | |
| Ethanolamine (ITS-X) | |
| Recombinant Human | Peprotech |
| LIF | |
| CHIR-99021 | Selleckchem |
| PD0325901 | Selleckchem |
| Recombinant Human | Peprotech |
| FGF-basic | |
| Recombinant | Peprotech |
| Human/Murine/Rat | |
| Activin A | |
| Emricasan | Selleckchem |
| Polyamine supplement | Sigma-Aldrich |
| 5 ml | |
| Antioxidant supplement, | Sigma-Aldrich |
| 5 ml | |
| trans-ISRIB, 10 mg | Tocris |
| Chroman 1 (HY15392), | Medchemexpress |
| 5 mg | |
| mTeSR Plus | STEMCELL Technologies |
| WH-4-023 | Tocris |
| endo-IWR 1 | Tocris |
| XAV-939 | Tocris |
| L-Ascorbic acid 2- | Sigma-Aldrich |
| phosphate | |
| AlbumiNZ Free Fatty | MP Biomedicals |
| Acid | |
| Dulbecco's phosphate | Corning |
| buffered saline | |
| (1X), no calcium, no | |
| magnesium (DPBS) | |
| DMEM/F12 | Gibco |
| GlutaMAX (100X) | Gibco, Cat. No. 35050-061 |
| MEM Non-Essential | Gibco |
| Amino Acids (100X) | |
| 2-Mercaptoethanol | Gibco |
| (1000X) | |
| Fetal Bovine Serum, | Gibco |
| heat inactivated | |

DEPOSITED DATA

| | |
|---|---|
| 8 cell and 16 cell | Lavagi et al.³¹ |
| Zygote, 2 cell, 8 cell, | Zhao et al.³⁰ |
| morula and blastocyst | |
| gastrulation embryos | Provided by Pfeffer et al.³² |
| In vivo blastocyst and in | Provided by Dr. Zongliang Jiang |
| vitro blastocyst | |
| Blastoid | This study |

SOFTWARE AND ALGORITHMS

| | |
|---|---|
| Cell Ranger (v 3.1.0) | http://www.10xgenomics.com/ |
| Seurat (v 3.1.4) | Stuart et al.³⁶ |
| Trim Galore (v 0.6.4) | http://www.bioinformatics.babraham.ac.uk/projects/trim_{—} |
| | galore/ |
| HISAT2 (v 2.1.0) | Kim et al.³⁷ |
| HTSeq-count (v 2.0.2) | Putri et al.³⁸ |
| clusterProfiler (v 3.14.3) | Wu et al.³⁹ |
| Monocle3 (v 0.2.3.0) | Cao et al.⁴⁰ |
| Scanpy (v 1.9.3) | Wolf et al.⁴¹ |
| Scikit-learn (v 1.2.1) | Pedregosa et al.⁴² |
| Imaris XT | Oxford Instruments |
| Code used in this study | https://github.com/anaorsi/CSC_{—} |
| | BovineBlastoids/tree/CSC |
| | https://zenodo.org/record/7792885#.ZCnFqi- |
| | B2Ak |

OTHER

| | |
|---|---|
| Aggre Well 400 | STEMCELL Technologies |
| Anti-Adherence Rinsing | STEMCELL Technologies |
| Dispase | STEMCELL Technologies |
| Accumax | Thermo Fisher |
| TrypLE ™ Express | Gibco |
| ProFreeze Freezing | Lonza, 12-769E |
| medium | |
| CoolCell | Corning |
| PureCoat ™ Amine 6 | Corning |
| Well Plate | |
| BD LSR II Flow | BD Biosciences |
| cytometer | |
| Nikon CSU-W1 | Nikon |
| spinning-disk super | |
| resolution | |
| by optical pixel | |
| reassignment (SoRa) | |
| Chromium Next GEM | 10X Genomics |
| Single Cell 3′ Kit v3.1 | |
| Chromium Next GEM | 10X Genomics |
| Chip G Single Cell Kit | |
| Chromium Controller & | 10X Genomics |
| Next GEM Accessory | |
| Kit | |
| Centrifuge HERMLEZ | HERMLE Labortechnik GmbH |
| 326 K | |
| Stereo Microscope | Nikon |
| SMZ800N | |
| Thermo Plate | TOKAI HIT |
| Micropipette Puller P-97 | Sutter Instruments |
| Micro Forge MF-900 | Narishige |
| ECHO Revolve | Echo |
| Nikon Eclipse Ts2 | Nikon |
| Inverted microscope | |
| Hypoxic humidified | NuAire |
| Incubator | |
| CO2 humidified | NuAire |
| Incubator | |
| 8 well μ-siles | Ibidi |

MEDIA FORMULATIONS

| | |
|---|---|
| FACL: N2B27 base, 1% | FACl (Yu, et al. Nature 2021)⁴ |
| BSA, 10 ng/ml LIF, | |
| 20 ng/ml Activin A, | |
| 20 ng/ml FGF2, 3 μM | |
| Chir99021 | |
| bEPSCs (3i + LAF): | bEPSCs IWXCL (Zhao, I, |
| mTeSR base, 1% BSA, | PNAS, 2021)¹⁵ |
| 10 ng/ml LIF, 20 ng/ml | |
| Activin A, WH-4-023 | |
| 0.3 μM, 1 μM | |
| Chir99021 20 ng/ml | |
| FGF2, 5 μM IWR1 | |
| and/or 5 μM XAV-939, | |
| Ascorbic acid (Vitamic | |
| C) 50 μg/ml | |
| LCDM: N2B27 base, | LCDM (Y, Yang; WU, J; J, |
| 1% BSA, 10 ng/ml LIF, 3 | Belmonte 2017)^{18, 17} |
| μM Chir99021, 2 μM | |
| Minocycline | |
| hydrochloride (M), | |
| 2 μM S)-(+)- | |
| Dimethindene maleate | |
| (DiM / D) | |
| NBFR: N2B27 base, 1% | NBFR Boglioti, Y; et al, |
| BSA, 20 ng/ml FGF2, | PNAS (2018)⁹ |
| 2.5 μM IWR1. Optional | |
| 20 ng/ml LIF and | |
| 50 ng/ml Activin A | |
| (NBFR + LA) | |
| tFACL + PD: N2B27 | This study |
| base, 1% BSA, 0.5x | |
| ITS-X, 20 ng/ml LIF, | |
| 10 ng/ml Activin A, | |
| 10 ng/ml FGF2, 0.3 μM | |
| PD0325901, 1 μM | |
| Chir99021 | |

| | |
|---|---|
| REAGENT or | |
| RESOURCE | IDENTIFIER |

ANTIBODIES

| | |
|---|---|
| Anti-CDX-2, Clone | AM392-5M; RRID: AB_2650531 |
| CDX2-88 antibody | |
| Human SOX17 Affinity | AF1924; RRID: AB_355060 |
| Purified | |
| Polyclonal Ab antibody | |
| Sox-2 (E-4) antibody | sc-365823; RRID: AB 10842165 |
| Anti-Human SOX2 | AN833-5M; RRID: not available |
| GATA3-human | sc-268; RRID: AB_2108591 |
| antibody | |
| Keratin 18 | SAB4501665; |
| | RRID: AB_10746153 |
| Anti-ZO-1 Monoclonal | 33-9100; RRID: AB_87181 |
| Antibody | |
| Alexa Fluor ® 594 | A12381; RRID: AB_2315633 |
| Phalloidin antibody | |
| Phospho-Stat3 (Tyr705) | 9145T; RRID: AB_2491009 |
| (D3A7) | |
| Donkey anti-rabbit IgG | A31573; RRID: AB_2536183 |
| (H + L) | |
| Antibody, Alexa Fluor ™ | |
| 647 | |
| Donkey anti-mouse IgG | A21202; RRID: AB_141607 |
| (H + L) | |
| Antibody, Alexa | |
| Fluor ™488 | |
| Donkey anti-Goat IgG | A-21432; RRID: AB_2535853 |
| (H + L) | |
| Antibody, Alexa Fluor ™ | |
| 555 | |

CHEMICALS, PEPTIDES, AND RECOMBINANT PROTEINS

| | |
|---|---|
| N2 supplement (100X) | Cat. No. 17502-048 |
| B27 supplement (50X) | Cat. No. 17504-044 |
| Insulin-Transferrin- | Cat. No. 51500-056 |
| Selenium- | |
| Ethanolamine (ITS-X) | |
| Recombinant Human | Cat. No. 300-05 |
| LIF | |
| CHIR-99021 | Cat. No. S1263 |
| PD0325901 | Cat. No. S1036 |
| Recombinant Human | Cat. No. 100-18B |
| FGF-basic | |
| Recombinant | Cat. No. 120-14E |
| Human/Murine/Rat | |
| Activin A | |
| Emricasan | Cat. No. 50-136-5234 |
| Polyamine supplement | Cat. No. P8483 |
| 5 ml | |
| Antioxidant supplement, | Cat. No. A1345 |
| 5 ml | |
| trans-ISRIB, 10 mg | Cat. No. 5284 |
| Chroman 1 (HY15392), | Cat. No. 502029121 |
| 5 mg | |
| mTeSR Plus | Cat. No. 100-1130 |
| WH-4-023 | Cat. No. 5413 |
| endo-IWR 1 | Cat. No. 3532 |
| XAV-939 | Cat. No. 3748 |
| L-Ascorbic acid 2- | Cat. No. A8960 |
| phosphate | |
| AlbumiNZ Free Fatty | Cat. No. 199899 |
| Acid | |
| Dulbecco's phosphate | Cat. No. 354277 |
| buffered saline | |
| (1X), no calcium, no | |
| magnesium (DPBS) | |
| DMEM/F12 | Cat. No. 11320-033 |
| GlutaMAX (100X) | Cat. No. 35050-061 |
| MEM Non-Essential | Cat. No. 11140-050 |
| Amino Acids (100X) | |
| 2-Mercaptoethanol | Cat. No. 21985-023 |
| (1000X) | |
| Fetal Bovine Serum, | Cat. No. A3840001 |
| heat inactivated | |

DEPOSITED DATA

| | |
|---|---|
| 8 cell and 16 cell | GEO: GSE99210 |
| Zygote, 2 cell, 8 cell, | SRA: PRJNA727165 |
| morula and blastocyst | |
| gastrulation embryos | GEO: GSE215409 |
| In vivo blastocyst and in | GEO: GSE215409 |
| vitro blastocyst | |
| Blastoid | GEO: GSE221248 |

SOFTWARE AND ALGORITHMS

| | |
|---|---|
| Cell Ranger (v 3.1.0) | https://github.com/10XGenomics/cellranger; |
| | RRID: SCR_017344 |
| Seurat (v 3.1.4) | https://satijalab.org/seurat/articles/get_{—} |
| | started.html; RRID: SCR_016341 |
| Trim Galore (v 0.6.4) | http://www.bioinformatics.babraham.ac.uk/projects/trim_{—} |
| | galore/; |
| | RRID: SCR_011847 |
| HISAT2 (v 2.1.0) | http://daehwankimlab.github.io/hisat2/; |
| | RRID: SCR_015530 |
| HTSeq-count (v 2.0.2) | https://htseq.readthedocs.io/en/master/htseqcount.html; |
| | RRID: SCR_011867 |
| clusterProfiler (v 3.14.3) | http://bioconductor.org/packages/release/bioc/html/clusterProfiler.html; |
| | RRID: SCR_016884 |
| Monocle3 (v 0.2.3.0) | https://cole-trapnell-lab.github.io/monocle3/; |
| | RRID: SCR_018685 |
| Scanpy (v 1.9.3) | https://github.com/scverse/scanpy |
| Scikit-learn (v 1.2.1) | https://scikit-learn.org/stable/ |
| Imaris XT | https://imaris.oxinst.com/products/imaris- |
| | for-cell-biologists |
| Code used in this study | https://doi.org/10.5281/zenodo.7792885 |

OTHER

| | |
|---|---|
| Aggre Well 400 | Cat. No. 34,415 |
| Anti-Adherence Rinsing | Cat. No. 07,010 |
| Dispase | Cat. No. 07913 |
| Accumax | Cat. No. 00-4666-56 |
| TrypLE ™ Express | Cat. No. 12605036 |
| ProFreeze Freezing | Cat. No. 12-769E |
| medium | |
| CoolCell | Cat. No. 432000 |
| PureCoat ™ Amine 6 | Cat. No. 354721 |
| Well Plate | |
| BD LSR II Flow | N/A |
| cytometer | |
| Nikon CSU-W1 | N/A |
| spinning-disk super | |
| resolution | |
| by optical pixel | |
| reassignment (SoRa) | |
| Chromium Next GEM | Cat. No. 1000269 |
| Single Cell 3′ Kit v3.1 | |
| Chromium Next GEM | Cat. No. 1000127 |
| Chip G Single Cell Kit | |
| Chromium Controller & | Cat. No. 000204 |
| Next GEM Accessory | |
| Kit | |
| Centrifuge HERMLEZ | Z 326 K |
| 326 K | |
| Stereo Microscope | SMZ800N |
| SMZ800N | |
| Thermo Plate | N/A |
| Micropipette Puller P-97 | N/A |
| Micro Forge MF-900 | N/A |
| ECHO Revolve | N/A |
| Nikon Eclipse Ts2 | N/A |
| Inverted microscope | |
| Hypoxic humidified | N/A |
| Incubator | |
| CO2 humidified | N/A |
| Incubator | |
| 8 well μ-siles | Cat. No. 80821 |

MEDIA FORMULATIONS

| | |
|---|---|
| FACL: N2B27 base, 1% | https://doi.org/10.1038/s41586-021-03356-y |
| BSA, 10 ng/ml LIF, | |
| 20 ng/ml Activin A, | |
| 20 ng/ml FGF2, 3 μM | |
| Chir99021 | |
| bEPSCs (3i + LAF): | https://doi.org/10.1073/pnas.2018505118 |
| mTeSR base, 1% BSA, | |
| 10 ng/ml LIF, 20 ng/ml | |
| Activin A, WH-4-023 | |
| 0.3 μM, 1 μM | |
| Chir99021 20 ng/ml | |
| FGF2, 5 μM IWR1 | |
| and/or 5 μM XAV-939, | |
| Ascorbic acid (Vitamic | |
| C) 50 μg/ml | |
| LCDM: N2B27 base, | https://doi.org/10.1016/j.cell.2017.02.005 |
| 1% BSA, 10 ng/ml LIF, 3 | |
| μM Chir99021, 2 μM | |
| Minocycline | |
| hydrochloride (M), | |
| 2 μM S)-(+)- | |
| Dimethindene maleate | |
| (DiM / D) | |
| NBFR: N2B27 base, 1% | https://doi.org/10.1073/pnas.1716161115 |
| BSA, 20 ng/ml FGF2, | |
| 2.5 μM IWR1. Optional | |
| 20 ng/ml LIF and | |
| 50 ng/ml Activin A | |
| (NBFR + LA) | |
| tFACL + PD: N2B27 | N/A |
| base, 1% BSA, 0.5x | |
| ITS-X, 20 ng/ml LIF, | |
| 10 ng/ml Activin A, | |
| 10 ng/ml FGF2, 0.3 μM | |
| PD0325901, 1 μM | |
| Chir99021 | |

### Experimental Model and Subject Details

### Cell Lines and Culture Conditions

All cell lines used in this study were cultured at 37° C. in a 5% CO2 humidified incubator. Bovine EPSCs and TSCs were cultured on 0.1% gelatin-coated dishes and a layer of inactivated mouse embryonic fibroblasts (iMEF) at 5×10⁵ cells per cm2. All cell lines were periodically tested for mycoplasma contamination via PCR. Cell lines were authenticated by genomic PCR, RT-qPCRs, immunostaining, RNA-seq and/or in vitro differentiation.

### Bovine EPSCs Stem Cells Culture

Bovine female EPSCs¹⁵, generated via culture adaptation of bovine ESCs (derived and cultured in the FRI/NBFR [FGF+IWR1] condition^{9,10}) in an bovine EPSC culture (3i+LAF) 15: mTeSR base, 1% BSA, 10 ng/ml LIF, 20 ng/ml Activin A, WH-4-023 0.3 μM, 1 μM Chir99021 20 ng/ml FGF2, 5 μM IWRI and/or 5 μM XAV-939, Ascorbic acid (Vitamin C) 50 μg/ml. Bovine ESCs were adapted to the bEPSC (3i+LAF) condition for a minimum of 5 passages until doomed colony morphology was visible. N2B27 basal medium was prepared by adding 1×N2 supplement (Gibco), 1×B27 supplement (Gibco), 1× GlutaMAX, 1×NEAA (Gibco), and 2-mercaptonethanol (Gibco) (final concentration 0.1 mM) to 1:1 (vol/vol) mixture of DMEM/F12 (Gibco) and neurobasal medium (Gibco). Upon passaging, cells were washed with 1×PBS and dissociated with TrypLE (Thermo Fisher) for 3 minutes at 37° C.; cells were then collected with 0.05% BSA in DMEM-F12 (Thermo Fisher) and centrifuged at 1000×g for 3 minutes and resuspended in 1 ml of media per 9.6 cm². Each passage cells were counted using Countess II (Thermo Fisher) and plated at a density of 30,000 cells/cm², at this plating ratio cells were passaged every 4 days. Upon plating, cells were treated with either (Y-27632) or the CEPT cocktail⁴³ (50 nM chroman-1 [C, Tocris], 5 mM emricasan [E, Selleckchem], 0.7 μM trans-ISRIB [T, Tocris], and 1× polyamine supplement [P, Thermo]) during the first 12 h after passaging. Fresh culture media was added every day. Cells were cryopreserved in CoolCell freezing containers (Corning), in bEPSC media with 10% DMSO at 0.5×10\hat{ }6 cells per ml and stored in liquid nitrogen the following day. Detailed descriptions of media are in the key resources table.

### Bovine TSCs Stem Cell Culture

Bovine male TSCs¹⁸ were derived and cultured in LCDM (hLIF, CHIR99021, DiM and MiH) media with slight modifications (N2B27 base, 1% BSA, 10 ng/ml LIF, 3 μM Chir99021, 2 μM Minocycline hydrochloride (M), 2 μM (S)-(+)-Dimethindene maleate (D). During passaging cells were treated with Accumax (Thermo Fisher) or Dispase (STEMCELL Technologies) for 5 minutes at 37° C. (no PBS wash), cells were collected with the same volume of bTSC medium and gently lifted off the plate using a wide opening p100 pipette tip and gentle force. Cells were split at a 1:3 ratio and plated on iMEFs with CEPT. Only 1 ml of media was plated in a 6 well for the first 24 h to facilitate bTSCs attachment. bTSCs do not survive well after single-cell dissociation and tend to form trophospheres if not plated correctly. These steps are critical for long term culture and expansion of bTSCs. Cells were cryopreserved in CoolCell freezing containers (Corning) in 45% LCDM 45% FBS and 10% DMSO or ProFreeze Freezing medium (Lonza, 12-769E) at 2×10\hat{ }6 cells per ml.

### Animals

Cross breed (Bos taurus×Bos indicus) non-lactating female cows with an average age of 3 years were used as recipients. Cows were housed in open pasture, and under constant care of farm staff.

### Method Details

### Blastoid Formation

For blastoid formation, EPSCs single-cells were collected as stated above. Bovine TSCs were washed with 1×PBS, dissociated with Trypsin for 10 minutes at 37° C., with constant pipetting every 2-3 minutes and inactivated with DMEM-F12 containing 10% fetal bovine serum (FBS). Cells were washed twice and on final resuspension in their normal culture media with 1×CEPT and 10 UI per ml of DNase I (Thermo Fisher). To deplete iMEF cells, collected cells were placed in precoated 12 well plates (Corning) with 0.1% gelatin and incubated for 15 minutes at 37° C. Single-cell dissociation was made by gentle but constant pipetting and by passing the cells through a glass capillary pulled to an inner diameter of 50-100 mm (micropipette puller. Sutter Instruments), hermetically attached to a p200 pipette tip. After single-cell dissociation, cells were collected and strained using a 70 μm (TSCs) and then a 37 μm cell strainers (EPSCs) (Corning). This same single-cell dissociation procedure was used for blastoids processing for 10× genomics. Cells were stained with 1× trypan blue and manually counted in a Neubauer chamber. Current protocol is optimized for 16 bEPSCs and 16 bTSCs per well in a-1200 well Aggrewell 400 microwell culture plate (Stemcell technologies) for 19,200 of each cell type per well. Each well was precoated with 500 ml of Anti-Adherence Rinsing Solution (Stemcell technologies) and spun for 5 minutes at 1500 rcf. Wells were rinsed with 1 ml of PBS just before aggregation. An appropriate number of cells for the wells to be aggregated was centrifuged at 1000×g for 3 minutes and resuspended in 1 ml of tFACL+PD media (N2B27 base, 1% BSA, 0.5×ITS-X, 20 ng/ml LIF, 10 ng/ml Activin A, 10 ng/ml FGF2, 0.3 μM PD0325901, 1 μM Chir99021) per well, supplemented with 1×CEPT. To ensure even distribution of the cells within each microwell, cells were gently mixed by pipetting with a P200 pipette, then the plate was centrifuged at 1300 ref for 2 minutes and put in a humidified incubator at 37° C. with 5% CO2 and 5% Oxygen (NuAire). As MEK inhibition inhibits hypoblast differentiation a gradual decrease can be done if higher numbers of hypoblast cells are desired from 0.3 to 0.125 μM. It is important to have viable. MEF free, cell debris free, and evenly distributed cells as any of these factors can negatively affect blastoid formation.

### In Vitro Fertilization

Bovine IVF was performed as previously described⁴⁴ with modifications. Briefly oocytes were collected at a commercial abattoir (DeSoto Biosciences) and shipped in an MOFA metal bead incubator (MOFA Global) at 38.5° C. overnight in sealed sterile vials containing 5% CO2 in air-equilibrated Medium 199 with Earle's salts (Thermo Fisher), supplemented with 10% fetal bovine serum (Hyclone), 1% penicillin-streptomycin (Invitrogen), 0.2-mM sodium pyruvate, 2-mM L-glutamine (Sigma), and 5.0 mg/ml of Folltropin (Vetoquinol). The oocytes were matured in this medium for 22 to 24 hours. Matured oocytes were washed twice in warm Tyrode lactate (TL) HEPES supplemented with 50 mg/mL of gentamicin (Invitrogen) while being handled on a stereomicroscope (Nikon) equipped with a 38.5° C. stage warmer. In vitro fertilization was conducted using a 2-hour pre-equilibrated IVF medium modified TL medium supplemented with 250-mM sodium pyruvate, 1% penicillin-streptomycin, 6 mg/mL of fatty acid-free BSA (Sigma), 20-mM penicillamine, 10-mM hypotaurine, and 10 mg/mL of heparin (Sigma) at 38.5 C, 5% CO2 in a humidified air incubator. Frozen semen (Bovine-elite) was thawed at 35° C. for 1 minute, then separated by centrifugation at 200×g for 20 minutes in a density gradient medium (Isolate, Irvine Scientific) 50% upper and 90% lower. Supernatant was removed; sperm pellet was resuspended in 2-mL modified Tyrode's medium and centrifuged at 200 g for 10 minutes to wash. The sperm pellet was removed and placed into a warm 0.65-mL microtube before bulk fertilizing in Nunc four-well multidishes (VWR) containing up to 50 matured oocytes per well at a concentration of 1.0×10\hat{ }6 sperm/mL. 18 hours after insemination, oocytes were cleaned of cumulus cells by constant pipetting for 3-minutes in vortex in 100 ml drop of TL HEPES with 0.05% Hyaluronidase (Sigma), washed in TL HEPES, and then cultured in 500 ml of IVC media (IVF-Biosciences) supplemented with 0.5×N2B27 (Thermo Fisher) and FLI²¹ under mineral oil (Irvine Scientific) cultured until the blastocyst stage. Cleavage rates were recorded on Day 2, and viable embryos were separated from nonviable embryos. Blastocyst rates were recorded on Day 8 after IVF.

### Immunofluorescent Staining

Samples (Cells, single-cells, blastoids and blastocysts) were fixed with 4% paraformaldehyde (PFA) in 1×DPBS with 0.1% PVA for 20 min at room temperature, washed in wash buffer (0.1% Triton X-100, 5% BSA in 1×DPBS) for 15 minutes and permeabilized with 0.1-1% Triton X-100 in PBS for 1 h. For phosphor-specific antibodies samples were treated with 0.5% SDS for 1 h. Samples were then blocked with blocking buffer (PBS containing 5% Donkey serum, 5% BSA, and 0.1% Triton X-100) at room temperature for 1 h, or overnight at 4° C. Because of the large number of blastoids, to facilitate processing blastoids were gently washed out of the aggrewell plate and separated from cell debris using a 100 μM reversible strainer (Stem cells), blastoids were then placed in a 70 μm strainer (Corning) in a 6 well plate containing wash buffer, and the strainer was moved from one well to another between steps. Primary antibodies were diluted in blocking buffer according to key resources table. Blastoids were incubated in primary antibodies in 96 wells for 2 h at room temperature or overnight at 4° C. Samples were washed three times for 15 minutes with wash buffer, and incubated with fluorescent-dye conjugated secondary antibodies (AF-488, AF-555 or AF-647, Invitrogen) diluted in blocking buffer (1:300 dilution) for 2 h at room temperature or overnight at 4° C. Samples were washed three times with PBS-T. Finally, cells were counterstained with 300 nM 40,6-diamidino-2-phenylindole (DAPI) solution at room temperature for 20 min. Phalloidin was directly stained along with other secondary antibodies in the blocking buffer.

### Imaging

Phase contrast images were taken using a hybrid microscope (Echo Laboratories, CA) equipped with objective ×2/0.06 numerical aperture (NA) air, ×4/0.13 NA air, ×10/0.7 NA air and 20×/0.05 NA air. Fluorescence imaging was performed on 8 well μ-siles (Ibidi) on a Nikon CSU-W1 spinning-disk super-resolution by optical pixel reassignment (SoRa) confocal microscope with objectives ×4/0.13 NA, a working distance (WD) of 17.1 nm, air; 320/0.45 NA, WD 8.9-6.9 nm, air; 340/0.6 NA, WD 3.6-2.85 nm, air. In vitro growth blastocyst was imaged in a glass slide under a coverslip using a Keyence BZ-X810 scope.

### Imaging Analysis

Imaging experiments were repeated at least twice, with consistent results. In the figure captions, n denotes the number of biological repeats. Raw images were first processed in Fiji⁴⁵ to create maximal intensity projection (MIP) and an export of representative images. Nuclear segmentation was performed in Ilastik. MIP images and segmentation masks were processed in MATLAB (R2022a) using custom code, available in a public repository. Nuclear localized fluorescence intensity was computed for each cell in each field, and the value was then normalized to the DAPI intensity of the same cell. Intensity values of all cells were plotted as mean±s.d. Lineage cell number quantification was made using Imaris (v10, Oxford) XT module and spots colocalization tool. Total number of cells was calculated based on DAPI spots and spots for each channel were cleared if not overlapping DAPI spots. Hypoblast cells were calculated as SOX17 or SOX17 and SOX2 co-localized spots. Trophectoderm cells were calculated from CDX2 only, CDX2, and SOX17.

### Flow Cytometry

Blastoids were collected under a stereo microscope and single-cell dissociated as stated above for the TSCs. Strained single-cells were processed as stated above for immunofluorescent staining performing wash steps in 1.5 ml Eppendorf tubes on a 90° centrifuge. Flow cytometry was performed using the appropriate unstained and single stain controls in a DBiosciences LSR II flow cytometer and analyzed using Flow Jo. Gating Strategy is shown in FIG. 8, panel K and panel L.

### In Vitro Growth

Prior to use for bovine blastoid culture, the water beads inside the humidity chamber of the ClinoReactor (CelVivo), were hydrated with sterile water (Corning) overnight at 4° C. Once hydrated and the growth chamber was filled with N2B27 basal media, and the reactor chamber was equilibrated for 1 h at 37° C. before exchanging for culture media. For rotating-culture blastoids were collected at day 4 post aggregation and placed in pre-equilibrated ClinoReactors in 10 ml of tFACL+PD03 media and 1×CEPT (key resources table). ClinoReactors were placed in the ClinoStar incubator at 37° C. with a gas mix of 5% CO₂, 5% O₂ and air. The rotation speed was set between 10 and 12 rpm and was lowered progressively as the blastoids expanded. Optimal growth conditions were achieved by exchanging media every four days. Blastoid and blastocysts growth was also tested on N2B27 with rock inhibitor (Y27632) and activin A as reported in Ramos-Ibeas et al.⁴⁶ (FIG. 8, panels S-W). Blastoids could also grow in N2B27 with 20 ng of FGF4, 20 ng of FGF2 and Activin A (FFA).

### Embryo Transfer

Surrogate cows were synchronized with an intramuscular (IM) injection of ovulation-inducing gonadotropin-release hormone (GnRH, Fertagyl), followed by a standard 7-day vaginal controlled drug internal release (CIDR) of progesterone. Upon CIDR removal, one dose of prostaglandin (Lutalyse) was administered. 48 hours after CIDR removal another dose of GnRH was administered via IM injection. A cohort of 15-20 bovine blastoids or 12-15 control IVF blastocysts were loaded into 0.5 mL straws in prewarmed Holding medium (ViGro) and transferred non-surgically to the uterine horn ipsilateral to the ovary with the corpus luteum (CL) as detected by transrectal ultrasound. 7 days after transfer, blastoids were recovered by standard non-surgical flush with lactated ringers' solution supplemented with 1% fetal bovine serum. All recipients were treated with prostaglandin (Lutalyse) after flushing.

### Quantitative Measurement of Bovine IFN-Tau in Blood

Blood samples from surrogate and controls were drawn from the coccygeal vein using serum separator tubes. The samples were immediately placed in refrigerator overnight before centrifugation for 15 minutes at 1000×g. IFNt in the serum was determined by Bovine Interferon-Tau ELISA Kit (CSB-E 16948B) according to manufacturer's protocol. Briefly, each well was added 100 μL standard or sample and incubated for 2 hours at 37° C. Then, liquid was removed and 100 μL Biotin-antibody (1×) was added to each well, incubating 1 hour at 37° C. After aspirating the wells, 200 μL Wash Buffer was used to wash the wells for three times. After last wash, the plate was inverted and blotted against clean paper towels to remove any remaining Wash Buffer. 100 μL HRP-avidin (1×) was added to each well and incubated for 1 hour at 37° C. 200 μL Wash Buffer was used to wash the wells for five times. 90 μL TMB Substrate was added and incubated for 20 minutes at 37° C. Protect from light. 50 μL Stop Solution was added to each well, gently tapping plate to ensure thorough mixing. The plate was measured using microplate reader set to 450 nm.

### Single-Cell RNA-Seq Library Generation

Bovine blastoids were single-cell dissociated and strained cells were prepared as stated adobe. Cells were washed in PBS containing 0.04% BSA and centrifuged at 90°×500 g for 5 min. Cell were resuspended in PBS containing 0.04% BSA at a single-cell suspension of 1,000 cells/μL. Cells were loaded into a 10× Genomics Chromium Chip following manufacturer instruction (10× Genomics, Pleasanton, CA, Chromium Next GEM Single Cell 3′ GEM, Library & Gel Bead Kit v3.1) and sequenced by Illumina NextSeq 500/550 sequencing systems (Illumina).

### Published Single-Cell Data Collection

We collected single-cell sequencing data from published literature for comparative analysis. Two Bovine IVF single-cell sequencing raw FASTQ data were downloaded from the GEO database, including 179 IVF cells³¹ sequenced using Smart-seq2 and 98 IVF cells³⁰ sequenced using STRT-seq. For the pseudo bulk analysis, bulk RNA sequencing data of gastrulation-stage embryos³² was also included.

### Pre-Processing Single-Cell Data

For 10× Genomics single-cell data, we used the Cell Ranger pipeline (v.3.1.0) with default parameters to generate the expression count matrix. The bovine reference genome and gene annotation file were downloaded from Ensembl database (UMD3.1) and generated by Cell Ranger mkfastq with default parameters. Seurat³⁶ (3.1.4) was used to single-cell quality control. To reduce multiplets and dead cells, we screened cells with expressed gene numbers between 2000 and 6000, unique molecular identifiers (UMIs) between 5000 and 30,000, and mitochondrial RNA genes counts below 15 percent.

For public Smart-seq2 and STRT-seq data, raw FASTQ reads were trimmed using Trim Galore (0.6.4, https://www.bioinformatics.babraham.ac.uk/projects/trim_galore/) with default parameters. In order to minimize processing differences, trimmed reads were aligned to the same genome reference (UMD3.1) by using HISAT2³⁷ (2.1.0) with default parameters. Read counts per gene were annotated by HTSeq-count³⁸ software (2.0.2) using the same gene annotation files (UMD3.1). Then, transcripts per million (TPM) were calculated to reduce gene length differences. Also, dead cells were removed by filter mitochondrial gene counts content below 15%.

### Normalization and Dimensionality Reduction

We used log-percentage value to normalize each single-cell expression matrix, which can reduce the bias of gene expression values caused by different sequencing depths and sequencing methods. In order to reduce the dimension of feature genes and improving the efficiency and accuracy of integration, the variance and mean of genes in each single-cell cohort were used to fit local polynomial regression and filter the top 2000 variable feature genes.⁴⁷

### Data Integration and Clustering

The Find Integration Anchors model in the Seurat package was used to find the similarity anchor structure between different single-cell data. Then, we completed the data integration according to the anchors information with 80 dimensions, 20 anchors, 40 candidate cells, and reciprocal PCA for dimensionality reduction (‘dims=1:80, k.anchor=20, k.filter=40, reduction=“rpca”’). Single cells were clustered using the shared nearest neighbor (SNN) modularity optimization-based clustering algorithm in Seurat package, with 90 Principal Component (PC) and 0.6 resolution. Then, Uniform manifold approximation and projection (UMAP) was used to reduce the dimensions and show the visualize figure with non-default parameters: ‘dims=1:90’.

### Pseudo Bulk Analysis

Clusters of blastoid cells were annotated according to the expression of marker genes. To generate pseudo bulk counts, total counts for each gene were summed for cells sharing a cluster. Genes expressed in less than 5% of all samples were excluded. Cells were normalized by total counts and log-transformed. Data was scaled and the PCA was calculated by Python package Scikit-learn.

For single-cell RNA-seq pseudo bulk data integration, blastoid cells were processed with Python package Scanpy. Cells were divided into 52 clusters and raw counts per gene from all cells sharing a cluster were summed. After this process, blastoid pseudo bulk samples contained approximately the same number of counts of embryo cells. Samples containing more than 4% of counts from mitochondrial genes or with number of genes per counts less than 2000 were excluded. The data was normalized, log-transformed and the top 4000 variable genes were kept for further analysis. Samples were scaled and Principal Component Analysis was carried out with Harmony Integrate, using dataset as key.

### Differentially Expressed Genes Analysis

Differentially expressed genes between clusters and groups were determined using the Scanpy rank genes groups tool, using a Wilcoxon rank-sum test.

### Matrix Plot Heatmap Data Graphical Representation

Graphs were made using the Scanpy matrixplot function according to predetermined lists gathered from the MSigDB significant signatures or published stem cell signaling gene lists. Graphs were made by grouping pseudo bulk data by developmental stage and lines annotation.

### Gene Function Annotation

Gene ontology (GO)⁴⁸ terms and Kyoto encyclopedia of genes and genomes (KEGG)¹⁹ pathways enrichment were performed using clusterProfiler³⁹ (3.14.3; org.Bt.eg.db v 3.10.0) with parameter: ‘pvalueCutoff=0.05’.

### Pseudotime Construction

Monocle3⁴⁰ (0.2.3.0) was used for pseudotime analysis, with the UMI matrix and UMAP embedding matrix generated by Seurat as input. Cell pseudotime trend was learnt by using cells in all clusters to generate a single and acyclic structure graph (‘use_partition=F, close_loop=F’).

### Datasets Used

8 cell and 16 cell from GSE99210.31 Zygote, 2 cell, 8 cell, morula and blastocyst from PRJNA727165.³⁰ Raw unprocessed data of gastrulation embryos was obtained.³² In vivo blastocyst and in vitro blastocyst1 datasets were obtained. Bovine blastoid single-cell raw and processed data have been deposited in the Gene Expression Omnibus under accession code (GSE221248).

### REFERENCES CITED IN THIS EXAMPLE

1. Rivron, N. C., Frias-Aldeguer, J., Vrij. E. J., Boisset, J.-C., Korving, J., Vivié. J., Truckenmuller. R. K., van Oudenaarden. A., van Blitterswijk. C. A., and Geijsen. N. (2018). Blastocyst-like structures generated solely from stem cells. Nature 557, 106-111. https://doi.org/10.1038/s41586-018-0051-0).
2. Sozen. B . . . . Cox. A. L., De Jonghe, J., Bao. M., Hollfelder, F., Glover, D. M., and Zernicka-Goetz. M. (2019). Self-organization of mouse stem cells into an extended potential blastoid. Dev. Cell 51, 698-712.e8.
3. Li, R., Zhong. C., Yu, Y., Liu, H., Sakurai, M., Yu, L., Min, Z., Shi, L., Wei, Y., Takahashi, Y., et al. (2019). Generation of blastocyst-like structures from mouse embryonic and adult cell cultures. Cell 179, 687-702.e18. https://doi.org/10.1016/j.cell.2019.09.029.
4. Yu, L., Wei, Y., Duan, J., Schmitz, D. A., Sakurai, M., Wang, L., Wang, K., Zhao. S., Hon, G. C., and Wu. J. (2021). Blastocyst-like structures generated from human pluripotent stem cells. Nature 591, 620-626. https://doi.org/10.1038/s41586-021-03356-y
5. Yanagida, A., Spindlow, D., Nichols, J., Dattani, A., Smith, A., and Guo, G. (2021). Naive stem cell blastocyst model captures human embryo lineage segregation. Cell Stem Cell 28, 1016-1022.e4. https://doi.org/10.1016/j.stem.2021.04.031.
6. Liu, X., Tan, J. P., Schröder, J., Aberkane, A., Ouyang, J. F., Mohenska, M., Lim, S. M., Sun, Y. B. Y., Chen, J., Sun, G., et al. (2021). Modelling human blastocysts by reprogramming fibroblasts into iBlastoids. Nature 591, 627-632. https://doi.org/10.1038/s41586-021-03372-y.
7. Kagawa, H., Javali, A., Khoci, H. H., Sommer, T. M., Sestini, G., Novatchkova, M., Scholte op Reimer, Y., Castel, G., Bruneau, A., Maenhoudt, N., et al. (2022). Human blastoids model blastocyst development and implantation. Nature 601, 600-605. https://doi.org/10.1038/s41586-021-04267-8.
8. Yu, L., Ezashi, T., Wei, Y., Duan, J., Logsdon, D., Zhan, L., Nahar, A., Arteaga, C. A. P., Liu. L., Stobbe. C., et al. (2022). Large scale production of human blastoids amenable to modeling blastocyst development and maternal-fetal crosstalk. https://doi.org/10.1101/2022.09.14.507946
9. Bogliotti, Y. S., Wu, J., Vilarino, M., Okamura, D., Soto, D. A., Zhong, C., Sakurai, M., Sampaio, R. V., Suzuki, K., Izpisua Belmonte, J. C., and Ross, P. J. (2018). Efficient derivation of stable primed pluripotent embryonic stem cells from bovine blastocysts. Proc. Natl. Acad. Sci. USA 115, 2090-2095. https://doi.org/10.1073/pnas. 1716161115.
10. Soto, D. A., Navarro, M., Zheng. C., Halstead. M. M., Zhou. C., Guiltinan, C., Wu, J., and Ross, P. J. (2021). Simplification of culture conditions and feeder-free expansion of bovine embryonic stem cells. Sci. Rep. 11, 11045. https://doi.org/10.1038/s41598-021-90422-0).
11. Kinoshita, M., Kobayashi, T., Planells, B., Klisch, D., Spindlow, D., Masaki, H., Bornelöv, S., Stirparo, G. G., Matsunari, H., Uchikura, A., et al. (2021). Pluripotent stem cells related to embryonic disc exhibit common selfrenewal requirements in diverse livestock species. Development 148, dev199901, https://doi.org/10.1242/dev. 199901.
12. Jiang, Y., Cai, N,N., An, X. L., Zhu, W. Q., Yang, R., Tang, B., Li, Z. Y., and Zhang, X. M. (2023). Naïve-like conversion of bovine induced pluripotent stem cells from Sertoli cells. Theriogenology 196, 68-78. https://doi.org/10.1016/j.theriogenology.2022.10.043.
13. Xiang, J., Wang, H., Zhang, Y., Wang, J., Liu, F., Han, X., Lu, Z., Li, C., Li, Z., Gao, Y., et al. (2021). LCDM medium supports the derivation of bovine extended pluripotent stem cells with embryonic and extraembryonic potency in bovine-mouse chimeras from iPSCs and bovine fetal fibroblasts. FEBS J. 288, 4394-4411. https://doi.org/10.1111/febs.15744.
14. Su, Y., Wang, L., Fan, Z., Liu, Y., Zhu, J., Kaback, D., Oudiz, J., Patrick, T., Yee, S. P., Tian, X. C., et al. (2021). Establishment of bovine-induced pluripotent stem cells. Int. J. Mol. Sci. 22, 10489. https://doi.org/10.3390/ijms221910489.
15. Zhao, L., Gao, X., Zheng, Y., Wang. Z., Zhao, G., Ren, J., Zhang, J., Wu, J., Wu, B., Chen, Y., et al. (2021). Establishment of bovine expanded potential stem cells. Proc. Natl. Acad. Sci. USA 118, e2018505118. https://doi.org/10.1073/pnas.2018505118.
16. Navarro. M., Soto, D. A., Pinzon, C. A., Wu. J., and Ross. P. J. (2020). Livestock pluripotency is finally captured in vitro. Reprod. Fertil. Dev. 32, 11-39.
17. Yang, Y., Liu, B., Xu, J., Wang, J., Wu, J., Shi, C., Xu, Y., Dong, J., Wang. C., Lai, W., et al. (2017). Derivation of pluripotent stem cells with in vivo embryonic and extraembryonic potency. Cell 169, 243-257.e25. https://doi.org/10.1016/j.cell.2017.02.005.
18. Wang, Y., Ming, H., Yu, L., Li, J., Zhu, L., Sun, H. X., Pinzon Arteaga, C., Wu, J., and Jiang, Z. (2023). Establishment of Bovine Trophoblast Stem Cells. Cell Reports 42. https://doi.org/10.1016/j.celrep.2023.112439.
19. Yu. L., Wei. Y., Sun, H. X., Mahdi, A. K., Pinzon Arteaga, C. A., Sakurai, M., Schmitz, D. A., Zheng, C., Ballard, E. D., Li, J., et al. (2021). Derivation of intermediate pluripotent stem cells amenable to primordial germ cell specification. Cell Stem Cell 28, 550-567.e12. https://doi.org/10.1016/j.stem.2020.11.003.
20. Wei, Y., Zhang, E., Yu. L., Ci, B., Guo, L., Sakurai, M., Takii, S., Liu, J., Schmitz., D. A., Ding. Y., et al. (2023). Dissecting embryonic and extra-embryonic lineage crosstalk with stem cell co-culture. https://doi.org/10.1101/2023.03.07.531525.
21. Stoecklein, K. S., Ortega, M. S., Spate, L. D., Murphy, C. N., and Prather, R. S. (2021). Improved cryopreservation of in vitro produced bovine embryos using FGF2, LIF, and IGF1. PLOS One 16, e0243727. https://doi.org/10.1371/journal.pone.0243727.
22. Turner, N., and Grose, R. (2010). Fibroblast growth factor signalling: from development to cancer. Nat. Rev. Cancer 10, 116-129. https://doi.org/10.1038/nrc2780.
23. Lavoie, H., Gagnon, J., and Therrien. M. (2020). ERK signalling: a master regulator of cell behaviour, life and fate. Nat. Rev. Mol. Cell Biol. 21, 607-632. https://doi.org/10.1038/s41580-020-0255-7.
24. Rossant, J., and Tam, P. P. L. (2017). New insights into early human development: lessons for stem cell derivation and differentiation. Cell Stem Cell 20, 18-28.
25. Canizo, J. R., Ynsaurralde Rivolta, A. E., Vazquez Echegaray, C., Suvá, M., Alberio, V., Aller. J. F., Guberman, A. S., Salamone, D. F., Alberio. R. H., and Alberio. R.
(2019). A dose-dependent response to MEK inhibition determines hypoblast fate in bovine embryos. BMC Dev. Biol. 19, 13. https://doi.org/10.1186/s12861-019-0193-9.
26. Sheikh, A. A., Hooda, O. K., Kalyan, A., Kamboj, A., Mohammed, S., Alhussien, M., Reddi, S., Shimray, P. G., Rautela, A., Pandita. S., et al. (2018). Interferon-tau stimulated gene expression: A proxy to predict embryonic mortality in dairy cows. Theriogenology 120, 61-67.
27. Hansen. P. J., and Tríbulo. P. (2019). Regulation of present and future development by maternal regulatory signals acting on the embryo during the morula to blastocyst transition-insights from the cow. Biol. Reprod. 101, 526-537. https://doi.org/10.1093/biolre/ioz030.
28. Brooks, K., and Spencer, T. E. (2015). Biological roles of interferon tau (IFNT) and Type I IFN receptors in elongation of the ovine Conceptus1. Biol. Reprod. 92, 47. https://doi.org/10.1095/biolreprod. 114.124156.
29. Bazer. F. W., and Thatcher, W. W. (2017). Chronicling the discovery of interferon tau. Reprod. Camb. Engl. 154, F11-F20. https://doi.org/10.1530/REP-17-0257.
30. Zhao. L., Long. C., Zhao. G., Su. J., Ren. J., Sun, W., Wang. Z., Zhang. J . . . . Liu, M., Hao, C., et al. (2022). Reprogramming barriers in bovine cells nuclear transfer revealed by single-cell RNA-seq analysis. J. Cell. Mol. Med. 26, 4792-4804. https://doi.org/10.1111/jcmm.17505.
31. Lavagi, I., Krebs, S., Simmet, K., Beck, A., Zakhartchenko, V., Wolf, E., and Blum. H. (2018). Single-cell RNA sequencing reveals developmental heterogeneity of blastomeres during major genome activation in bovine embryos. Sci. Rep. 8, 4071. https://doi.org/10.1038/s41598-018-22248-2.
32. Pfeffer, P. L., Smith. C. S., Maclean, P., and Berg, D. K. (2017). Gene expression analysis of bovine embryonic disc, trophoblast and parietal hypoblast at the start of gastrulation. Zygote 25, 265-278. https://doi.org/10.1017/S0967199417000090.
33. Corujo-Simon, E., Radley, A. H., and Nichols, J. (2022). Evidence implicating sequential commitment of the founder lineages in the human blastocyst by order of hypoblast gene activation. https://doi.org/10.1101/2022.12.08.519626.
34. Boroviak, T., Stirparo, G. G., Dietmann, S., Hernando-Herraez, I., Mohammed, H., Reik. W., Smith, A., Sasaki, E., Nichols, J., and Bertone. P. (2018). Single cell transcriptome analysis of human, marmoset and mouse embryos reveals common and divergent features of preimplantation development. Development 145, dev167833. https://doi.org/10.1242/dev.167833.
35. Luo, L., Shi, Y., Wang, H., Wang, Z., Dang, Y., Li, S., Wang, S., and Zhang, K. (2022). Base editing in bovine embryos reveals a species-specific role of SOX2 in regulation of pluripotency. PLOS Genet. 18, e1010307. https://doi.org/10.1371/journal.pgen.1010307.
36. Stuart, T., Butler, A., Hoffman, P., Hafemeister, C., Papalexi, E., Mauck, W. M., III, Hao, Y., Stoeckius, M., Smibert, P., and Satija. R. (2019). Comprehensive integration of single-cell data. Cell 177, 1888-1902.e21.
37. Kim, D., Paggi, J. M., Park, C., Bennett, C., and Salzberg, S. L. (2019). Graph-based genome alignment and genotyping with HISAT2 and HISAT-genotype. Nat. Biotechnol. 37, 907-915.
38. Putri. G. H., Anders, S., Pyl, P. T., Pimanda, J. E., and Zanini, F. (2022). Analysing high-throughput sequencing data in Python with HTSeq 2.0. Bioinformatics 38, 2943-2945.
39. Wu, T., Hu. E., Xu, S., Chen, M., Guo, P., Dai, Z., Feng, T., Zhou, L., Tang. W., Zhan. L., et al. (2021). clusterProfiler 4.0: A universal enrichment tool for interpreting omics data. Innovation (Camb) 2, 100141.
40. Cao, J., Spielmann, M., Qiu. X., Huang, X., Ibrahim, D. M., Hill, A. J., Zhang, F., Mundlos. S., Christiansen. L., and Steemers, F. J. (2019). The single-cell transcriptional landscape of mammalian organogenesis. Nature 566, 496-502.
41. Wolf, F. A., Angerer, P., and Theis, F. J. (2018). SCANPY: large-scale single-cell gene expression data analysis. Genome biology 19, 15. https://doi.org/10.1186/s13059-017-1382-0.
42. Pedregosa, F., Varoquaux, g., Gramfort, A., Michel, V., Thirion, B., Grisel. O., Blondel, M., Prettenhofer, P., Weiss, R., Dubourg, V., et al. (2011). Scikit-learn: Machine Learning in Python. Journal of Machine Learning Research. 12, 2825-2830.
43. Chen, Y., Tristan, C. A., Chen, L., Jovanovic, V. M., Malley, C., Chu. P. H., Ryu, S., Deng, T., Ormanoglu, P., Tao. D., et al. (2021). A versatile polypharmacology platform promotes cytoprotection and viability of human pluripotent and differentiated cells. Nat. Methods 18, 528-541. https://doi.org/10.1038/s41592-021-01126-2.
44. Golding, M. C., Snyder, M., Williamson, G. L., Veazey, K. J., Peoples, M., Pryor, J. H., Westhusin, M. E., and Long, C. R. (2015). Histone-lysine N-methyltransferase SETDB1 is required for development of the bovine blastocyst. Theriogenology 84, 1411-1422.
45. Schindelin, J., Arganda-Carreras, I., Frise, E., Kaynig. V., Longair. M., Pietzsch, T., Preibisch, S., Rueden, C., Saalfeld, S., Schmid, B., et al. (2012). Fiji: an open-source platform for biological-image analysis. Nat. Methods 9, 676-682. https://doi.org/10.1038/nmeth.2019.
46. Ramos-Ibcas, P., Lamas-Toranzo. I., Martínez-Moro, Á., de Frutos, C., Quiroga, A. C., Zurita, E., and Bermejo-Álvarez, P. (2020). Embryonic disc formation following post-hatching bovine embryo development in vitro. Reprod. Camb. Engl. 160, 579-589. https://doi.org/10.1530/REP-20-0243.
47. Hao, Y., Hao, S., Andersen-Nissen, E., Mauck, W. M., III, Zheng, S., Butler, A., Lee, M. J., Wilk, A. J., Darby, C., Zager, M., et al. (2021). Integrated analysis of multimodal single-cell data. Cell 184, 3573-3587.e29.
48. Gene Ontology Consortium (2021). The Gene Ontology resource: enriching a gold mine. Nucleic Acids Res. 49, D325-D334.
49. Kanehisa, M., Sato, Y., and Kawashima, M. (2022). KEGG mapping tools for uncovering hidden features in biological data. Protein Sci. 31, 47-53.

### Example 5

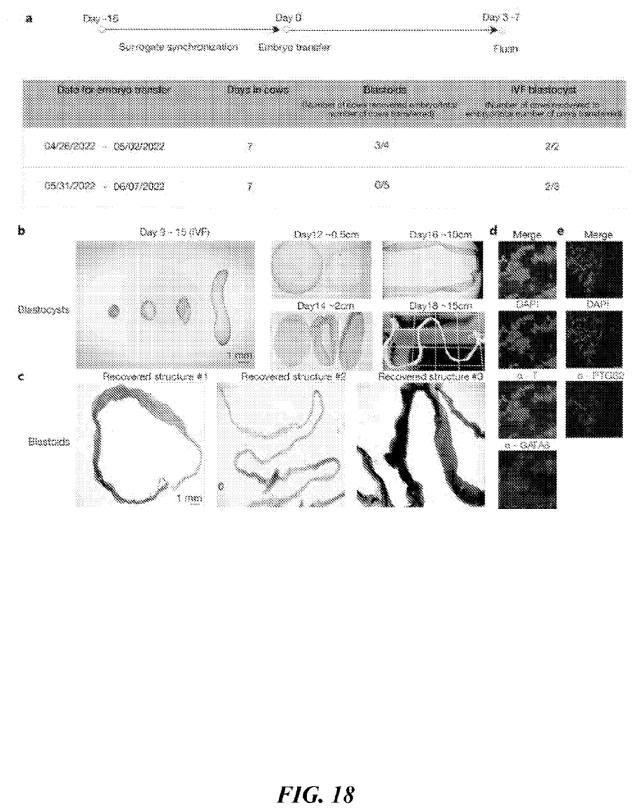

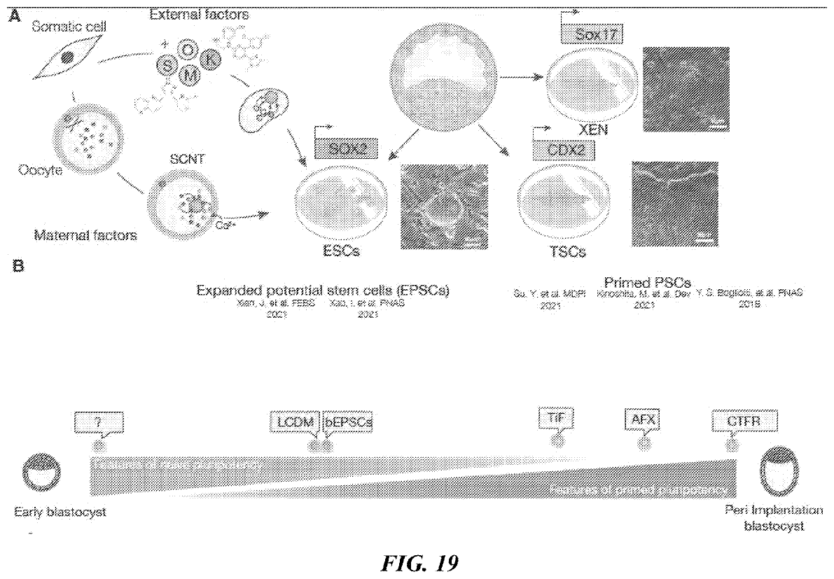

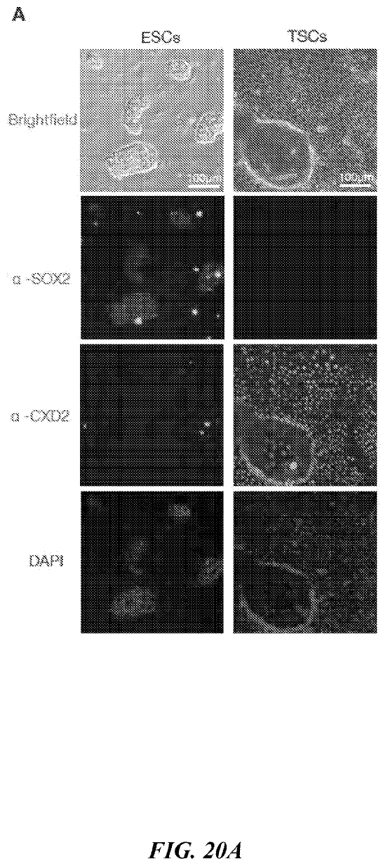

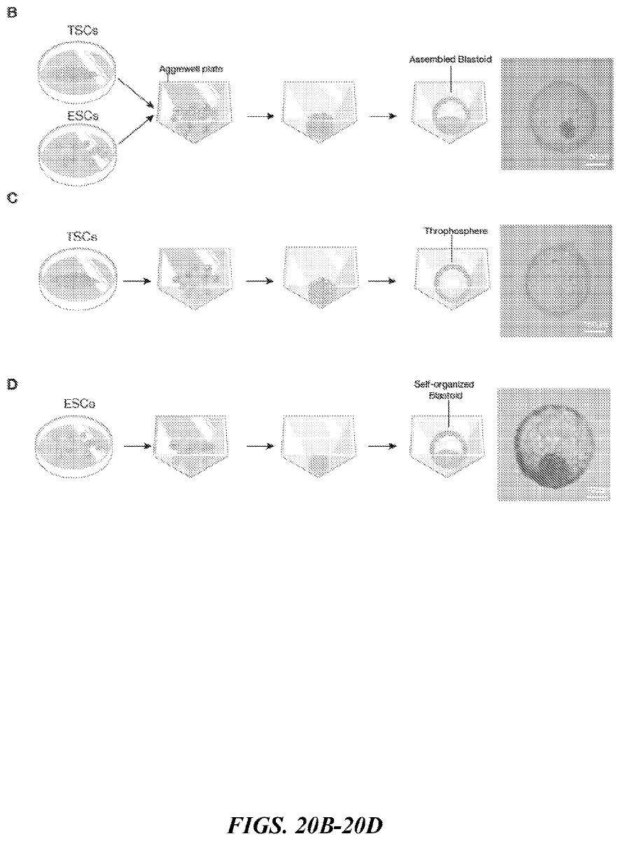

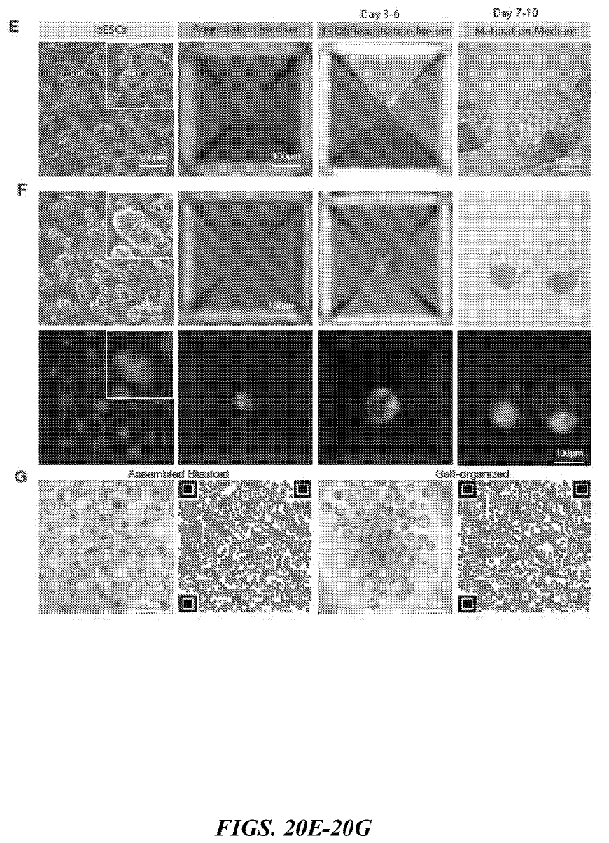

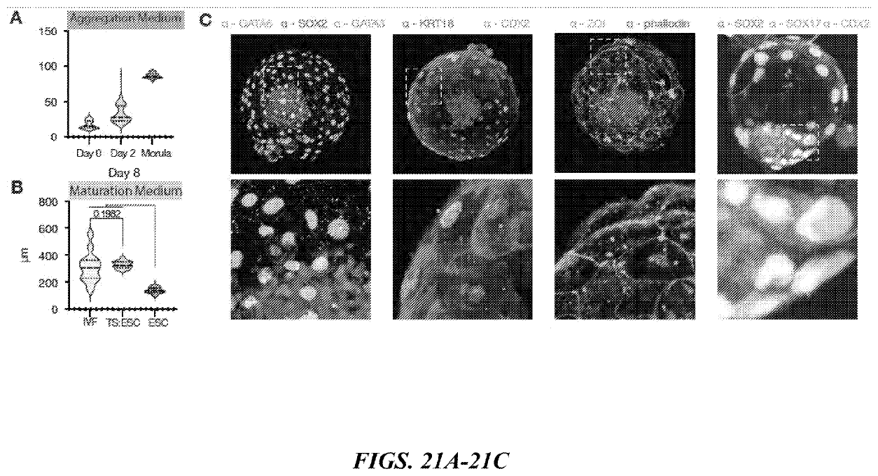

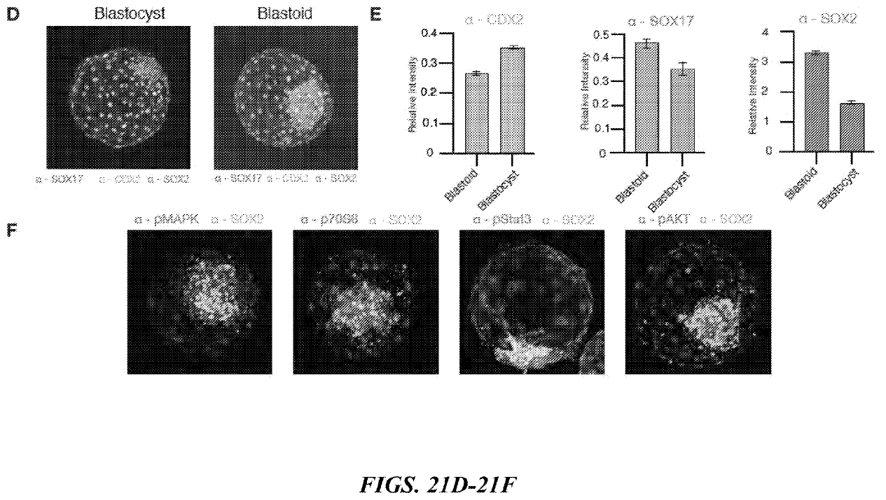

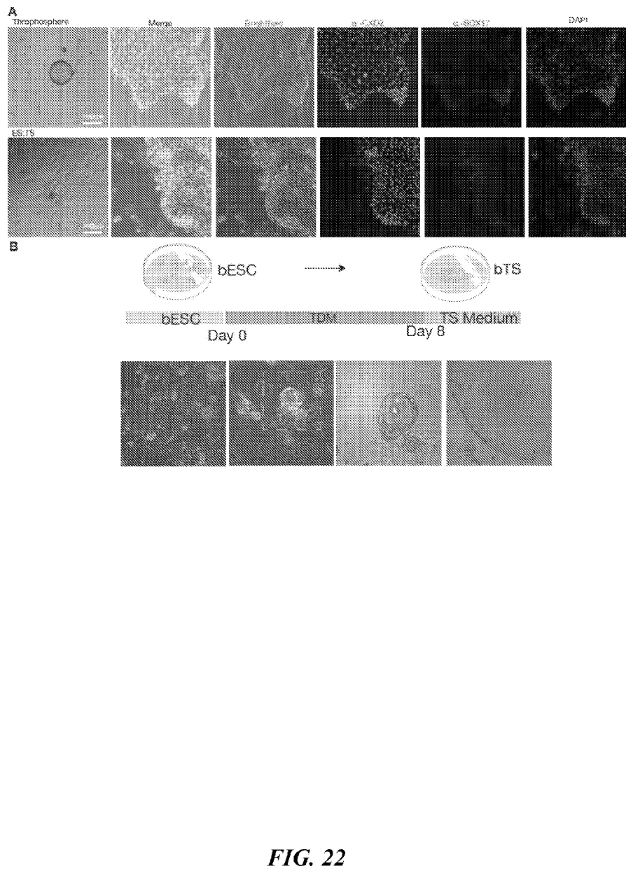

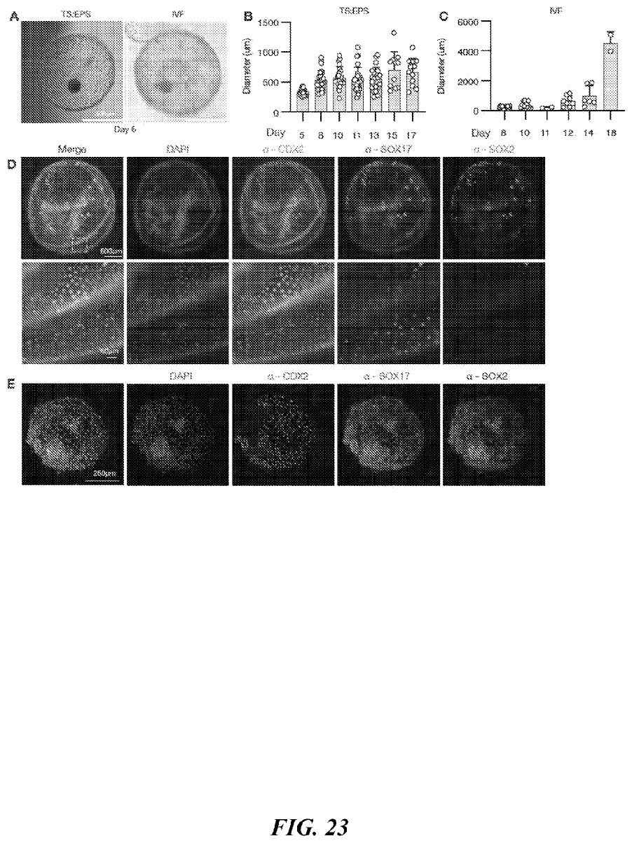

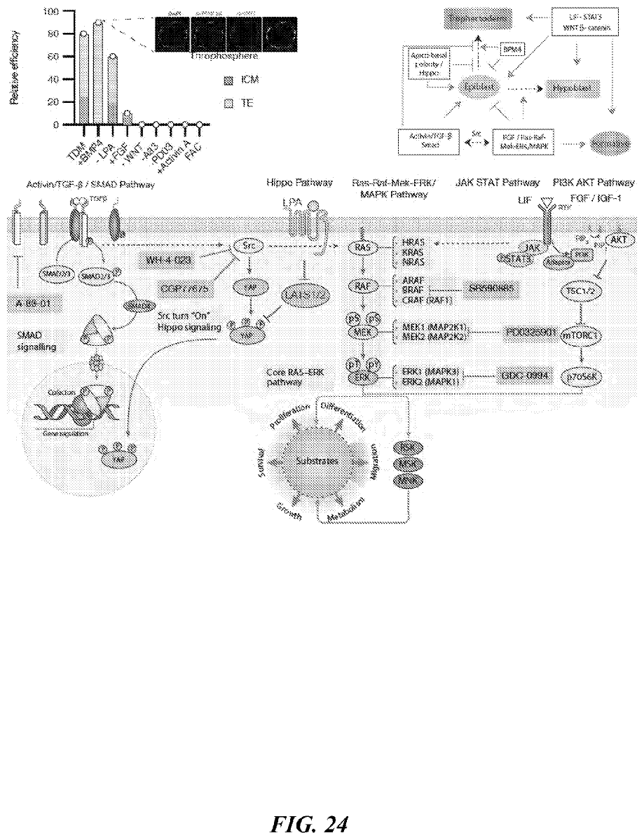

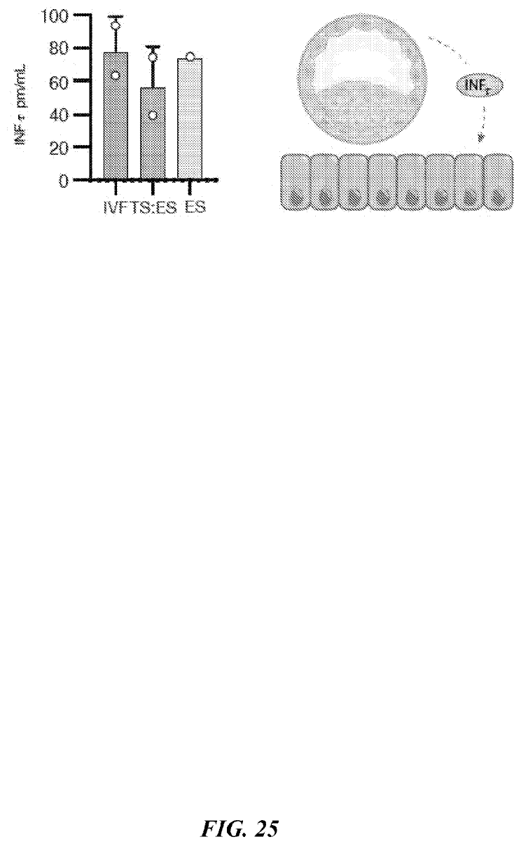

Although we didn't include a lot of embryo transfer (ET) data in the published CSC paper, we performed significant amount of work for ET of blastoids and control blastocysts, and embryo recovery. As summarized in the attached figure, after several rounds of blastoid transfer, we were able to recover 3 structures out of 15 transferred surrogates. These structures morphologically resembled days 16-18 in vivo bovine elongated embryos (FIG. 18). For downstream analysis, these structures were sectioned for immunostaining and stained positive for trophectoderm (PTGS2), hypoblast (GATA6) and mesoderm (T) markers. As you can see, these data are very encouraging and represent a critical step toward to using blastoids to model implantation. However, we feel the current ET data lacks statistical power and in-depth characterization as a result of several logistic and technical obstacles, e.g. lack of proper equipment in the farm where the ET and embryo collection were performed and technical difficulties in dealing with the large retrieved blastoid-derived structures (>15 cm). In cattle, the hatched blastocyst forms an ovoid conceptus between days 12 to 14 and is only about 2 mm in length on day 13. By day 14, the conceptus is about 6 mm, and the elongating bovine conceptus reaches a length of about 60 mm (6 cm) by day 16 and is 20 cm or more by day 19 (FIG. 18). To better analyze bovine blastoid-derived structures, earlier timepoints are preferred and additional ET experiments are needed.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

We claim:

1. An in vitro method for producing a blastocyst-like structure (blastoid), the method comprising:
culturing a bovine embryonic stem cell (bESC) or an induced pluripotent stem cell (iPSC) with a bovine trophoblast stem cell (TSC) for a length of time sufficient to observe the formation of a three-dimensional (blastoid) in a cell culture medium; and
incubating a SOX2-positive bESC or a SOX2-positive iPSC with a TSC expressing CDX2 for a length of time sufficient to observe formation of a SOX17-positive hypoblast in a cell culture medium,
wherein the medium comprises one or more factors selected from the group consisting of a STAT3 agonist, a SMAD2/3 agonist, a Wnt agonist, an inhibitor of the RAS-RAF MEK/ERK-MAPK pathway, a fibroblast growth factor, a PI3K-AKT agonist, or any combination thereof.

2. The method of claim 1, wherein the STAT3 agonist comprises human leukemia inhibitory factor (hLIF).

3. The method of claim 1, wherein the SMAD2/3 agonist comprises recombinant activin A (Activin A).

4. The method of claim 1, wherein the Wnt agonist comprises a glycogen synthase kinase-3 (GSK-3) inhibitor.

5. The method of claim 4, wherein the glycogen synthase kinase-3 (GSK-3) inhibitor comprises CHIR99021.

6. The method of claim 1, wherein the inhibitor of the MEK/ERK pathway comprises PD0325901.

7. The method of claim 1, wherein the fibroblast growth factor is FGF2.

8. The method of claim 1, wherein the PI3K-AKT agonist is insulin.

9. The method of claim 1, wherein the medium further comprises one or more factors selected from the group consisting of a ROCK kinase inhibitor, a pan-caspase inhibitor, an integrated stress response (ISR) inhibitor, and 1× polyamine supplement.

10. The method of claim 9, wherein the ROCK kinase inhibitor is cither chroman-1 or Y-27632.

11. The method of claim 9, wherein the pan-caspase inhibitor comprises emricasan.

12. The method of claim 9, wherein the integrated stress response (ISR) inhibitor comprises trans-ISRIB.

13. The method of claim 1, wherein the method further comprises isolating the resulting bovine blastoid from the culture.

14. The method of claim 1, wherein the medium comprises a STAT3 agonist, a SMAD2/3 agonist, a Wnt agonist, an inhibitor of the RAS-RAF-MEK/ERK-MAPK pathway, a fibroblast growth factor, and a PI3K-AKT agonist.

15. The method of claim 1, wherein the amount of the STAT3 agonist is about 20 ng/ml.

16. The method of claim 1, wherein the amount of the SMAD2/3 agonist is about 10 ng/ml.

17. The method of claim 1, wherein the amount of the Wnt agonist is about 1 μM.

18. The method of claim 1, wherein the amount of the inhibitor of the MEK/ERK pathway is about 0.3 μM.

19. The method of claim 1, wherein the amount of the fibroblast growth factor is about 10 ng/ml.

20. The method of claim 9, wherein the amount of the ROCK kinase inhibitor is about 50 nM.

21. The method of claim 9, wherein the amount of the pan-caspase inhibitor is about 5 μM.

22. The method of claim 9, wherein the amount of the integrated stress response (ISR) inhibitor is about 0.7 μM.

23. The method of claim 1, wherein the STAT3 agonist is human leukemia inhibitory factor (hLIF), the SMAD2/3 agonist is recombinant activin A (Activin A), the Wnt agonist is CHIR99021, the inhibitor of the RAS-RAF-MEK/ERK-MAPK pathway is PD0325901, the fibroblast growth factor is FGF2, and the PI3K-AKT agonist is insulin.

24. The method of claim 1, wherein the culturing is conducted in a microwell plate.

25. The method of claim 1, wherein the period of time is about 3 days.

26. The method of claim 1, wherein the trophoblast stem cell (TSC) comprises a bovine trophoblast stem cell (TSC) derived from a bovine blastocyst.

27. The method of claim 1, wherein the period of time is sufficient to transform the bovine embryonic stem cell and trophoblast stem cell into a bovine blastoid, wherein the blastoid mimics bovine blastocyst morphology, bovine blastocyst size, bovine blastocyst cell number, bovine blastocyst gene marker expression, bovine blastocyst lineage composition, bovine blastocyst allocation, or any combination thereof.

28. A method of assisted reproduction of farm animals, the method comprising:
transfer of the bovine blastoid produced by the method according to claim 1 in a synchronized surrogate uterus via embryo transfer.

29. The method of claim 28, wherein the bovine blastoid derives from an embryonic stem cell isolated from the subject.

30. The method of claim 28, wherein the subject is a surrogate.

31. A method of determining a drug toxicity, the method comprising:
Exposure of bovine blastoid produced by the method according to claim 1 with a drug, and detecting a toxicity indicator.

32. The method of claim 31, wherein the toxicity indicator comprises cell death, loss of blastoid cell organization, blastoid growth arrest, development arrest, or any combination thereof.

33. A bovine blastoid produced by the method according to claim 1.

34. The bovine blastoid of claim 33, wherein the blastoid recapitulates bovine embryo development in vitro and in vivo.

35. The bovine blastoid of claim 33, wherein the blastoid is capable of generating a viable embryo.

36. The bovine blastoid of claim 35, wherein the blastoid is capable of generating a viable embryo in a surrogate.

37. The bovine blastoid of claim 35, wherein generating a viable embryo is indicated by the presence of embryonic stem cells, extraembryonic stem cells, or both.

38. The bovine blastoid of claim 35, wherein generating a viable embryo is indicated by organization into peri-implantation embryo-like structures in vitro.

39. The bovine blastoid of claim 33, wherein the blastoid enters elongation in utero.

40. A cell culture comprising a population of bovine embryonic stem cells in a medium, wherein the medium comprises one or more factors selected from the group consisting of a STAT3 agonist, a SMAD2/3 agonist, a Wnt agonist, an inhibitor of the RAS-RAF-MEK/ERK-MAPK pathway, a fibroblast growth factor, a PI3K-AKT agonist, or any combination thereof.

41. The cell culture of claim 40, wherein the STAT3 agonist comprises human leukemia inhibitory factor (hLIF).

42. The cell culture of claim 40, wherein the SMAD2/3 agonist comprises recombinant activin A (Activin A).

43. The cell culture of claim 40, wherein the Wnt agonist comprises a glycogen synthase kinase-3 (GSK-3) inhibitor.

44. The cell culture of claim 43, wherein the glycogen synthase kinase-3 (GSK-3) inhibitor comprises CHIR99021.

45. The cell culture of claim 40, wherein the inhibitor of the MEK/ERK pathway comprises PD0325901.

46. The cell culture of claim 40, wherein the fibroblast growth factor is FGF2.

47. The cell culture of claim 40, wherein the PI3K-AKT agonist is insulin.

48. The cell culture of claim 40, wherein the medium further comprises one or more factors selected from the group consisting of a ROCK kinase inhibitor, a pan-caspase inhibitor, an integrated stress response (ISR) inhibitor, and 1× polyamine supplement.

49. The cell culture of claim 48, wherein the ROCK kinase inhibitor is either chroman-1 or Y-27632.

50. The cell culture of claim 48, wherein the pan-caspase inhibitor comprises emricasan.

51. The cell culture of claim 48, wherein the integrated stress response (ISR) inhibitor comprises trans-ISRIB.

52. The cell culture of claim 40, wherein the amount of the STAT3 agonist is about 20 ng/ml.

53. The cell culture of claim 40, wherein the amount of the SMAD2/3 agonist is about 10 ng/ml.

54. The cell culture of claim 40, wherein the amount of the Wnt agonist is about 1 μM.

55. The cell culture of claim 40, wherein the amount of the inhibitor of the MEK/ERK pathway is about 0.3 μM.

56. The cell culture of claim 40, wherein the amount of the fibroblast growth factor is about 10 ng/ml.

57. The cell culture of claim 48, wherein the amount of the ROCK kinase inhibitor is about 50 nM.

58. The cell culture of claim 48, wherein the amount of the pan-caspase inhibitor is about 5 μM.

59. The cell culture of claim 48, wherein the amount of the integrated stress response (ISR) inhibitor is about 0.7 μM.

60. The cell culture of claim 40, wherein the STAT3 agonist is human leukemia inhibitory factor (hLIF), the SMAD2/3 agonist is recombinant activin A (Activin A), the Wnt agonist is CHIR99021, the inhibitor of the RAS-RAF-MEK/ERK-MAPK pathway is PD0325901, the fibroblast growth factor is FGF2, and the PI3K-AKT agonist is insulin.

61. The cell culture of claim 40, wherein the culture is in a microwell plate.

62. The cell culture of claim 40, wherein the cell culture further comprises a population of trophoblast stem cells.

63. The cell culture of claim 62, wherein the population of trophoblast stem cells comprises a population of bovine trophoblast stem cells.
